(19) United States
(12) Patent Application Publication
(10) Pub. No.: US 2010/0081916 A1
(43) Pub. Date: Apr. 1, 2010

(54) Histological facilitation systems and methods

(75) Inventors: Roderick A. Hyde, Redmond, WA (US); Muriel Y. Ishikawa, Livermore, CA (US); Eric C. Leuthardt, St. Louis, MO (US); Jonathan E. Olson, Bellevue, WA (US); Dennis J. Rivet, Portsmouth, VA (US); Lowell L. Wood, JR., Bellevue, WA (US); Victoria Y.H. Wood, Livermore, CA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware.

(21) Appl. No.: 12/286,552

(22) Filed: Sep. 29, 2008

(51) Int. Cl. A61B 10/02; A61B 10/00

(52) U.S. Cl. 600/407; 600/564

(57) ABSTRACT

Systems, methods, and other modalities are described for generating or otherwise handling images or other data indicating (a) an extraction of chemically treated tissue frozen in vivo, (b) a treatment of a tissue sample in a chamber extended into tissue of an organism, and/or (c) cells to which an optical enhancement material was applied in vivo. Several contexts in which such indications facilitate histological evaluation are likewise described.

### SUMMARY

In one aspect, a method includes but is not limited to generating or otherwise obtaining device-detectable data indicating an extraction of chemically treated tissue frozen in vivo and transmitting a responsive or other evaluation of the device-detectable data.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to circuitry for generating or otherwise obtaining device-detectable data indicating an extraction of chemically treated tissue frozen in vivo and circuitry for transmitting a responsive or other evaluation of the device-detectable data. In addition to the foregoing, other system aspects are described in the claims, drawings, and text forming a part of the present disclosure.

In one aspect, a method includes but is not limited to generating or otherwise obtaining device-detectable data indicating a treatment of a tissue sample in a chamber extended into tissue of an organism and transmitting a responsive or other evaluation of the device-detectable data.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to circuitry for generating or otherwise obtaining device-detectable data indicating a treatment of a tissue sample in a chamber extended into tissue of an organism and circuitry for transmitting a responsive or other evaluation of the device-detectable data. In addition to the foregoing, other system aspects are described in the claims, drawings, and text forming a part of the present disclosure.

In one aspect, a method includes but is not limited to generating or otherwise obtaining sensor data indicating one or more cells to which an optical enhancement material was applied in vivo and transmitting a responsive or other evaluation of the device-detectable data.

In one or more various aspects, related systems include but are not limited to circuitry and/or programming for effecting the herein referenced method aspects; the circuitry and/or programming can be virtually any combination of hardware, software, and/or firmware configured to effect the herein referenced method aspects depending upon the design choices of the system designer.

In one aspect, a system includes but is not limited to circuitry for generating or otherwise obtaining sensor data indicating one or more cells to which an optical enhancement material was applied in vivo and circuitry for transmitting a responsive or other evaluation of the device-detectable data. In addition to the foregoing, other system aspects are described in the claims, drawings, and text forming a part of the present disclosure.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

In one or more various aspects, related systems include but are not limited to circuitry and/or programming for effecting herein-referenced method aspects; the circuitry and/or programming can be virtually any combination of hardware, software, and/or firmware configured to effect the herein-referenced method aspects depending upon the design choices of the system designer. In addition to the foregoing, various other method and/or system aspects are set forth and described in the teachings such as text (e.g., claims and/or detailed description) and/or drawings of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1-24 depict exemplary environments in which one or more technologies may be implemented.

FIG. 25 depicts a high-level logic flow of an operational process.

FIG. 26 depicts a high-level logic flow of an operational process.

FIG. 27 depicts a high-level logic flow of an operational process.

FIGS. 28-32 depict further environments in which one or more technologies may be implemented.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures suitable to operation. Electronic circuitry, for example, may manifest one or more paths of electrical current constructed and arranged to implement various logic functions as described herein. In some implementations, one or more media are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or otherwise invoking circuitry for enabling, triggering, coordinating, requesting, or otherwise causing one or more occurrences of any functional operations described above. In some variants, operational or other logical descriptions herein may be expressed directly as source code and compiled or otherwise invoked as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar mode(s) of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

In a general sense, those skilled in the art will recognize that the various embodiments described herein can be implemented, individually and/or collectively, by various types of electro-mechanical systems having a wide range of electrical components such as hardware, software, firmware, and/or virtually any combination thereof; and a wide range of components that may impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Those skilled in the art will also appreciate that examples of electromechanical systems include but are not limited to a variety of consumer electronics systems, medical devices, as well as other systems such as motorized transport systems, factory automation systems, security systems, and/or communication/computing systems. Those skilled in the art will recognize that electromechanical as used herein is not necessarily limited to a system that has both electrical and mechanical actuation except as context may dictate otherwise.

In a general sense, those skilled in the art will also recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). Those having skill in the art will recognize that the subject matter described herein may be implemented in an analog or digital fashion or some combination thereof.

Those skilled in the art will further recognize that at least a portion of the devices and/or processes described herein can be integrated into an image processing system. A typical image processing system may generally include one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system may be implemented utilizing suitable commercially available components, such as those typically found in digital still systems and/or digital motion systems.

Those skilled in the art will likewise recognize that at least some of the devices and/or processes described herein can be integrated into a data processing system. Those having skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

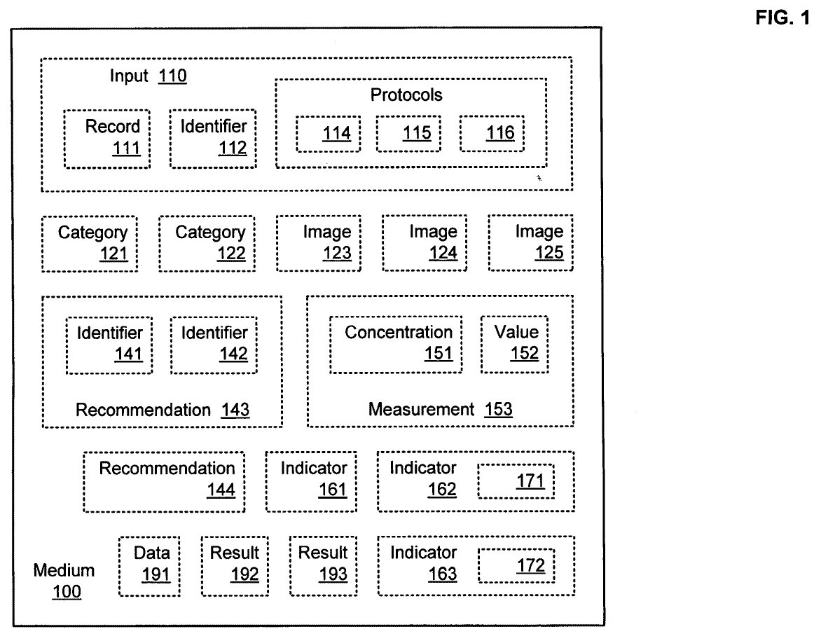

With reference now to FIG. 1, shown is a medical or veterinary system in which one or more technologies may be implemented. As described below, it includes a storage or transmission medium 100 bearing one or more instances of input 110; protocols 114, 115, 116; categories 121, 122; images 123, 124, 125; recommendations 143, 144; concentrations 151 or other values 152 representing one or more measurements 153; indicators 161, 162, 163 representing an identifier 171, time 172, or other such items; or other data 191 or results 192, 193 as described below. Such recommendations may include identifiers 141, 142 of known pathologies, differential diagnostic procedures, or other advice a consultant or expert system may provide. Such protocols may likewise be represented in human-readable and/or machine readable form in some embodiments, for example, or by various existing parametric representations. In some variants, for example, one or more displays or other physical media 100 are configured to bear (a) one or more earlier images 123, 124 depicting a cluster of cells to which an optical enhancement material was applied in vivo and (b) one or more later images 125 also depicting the cluster.

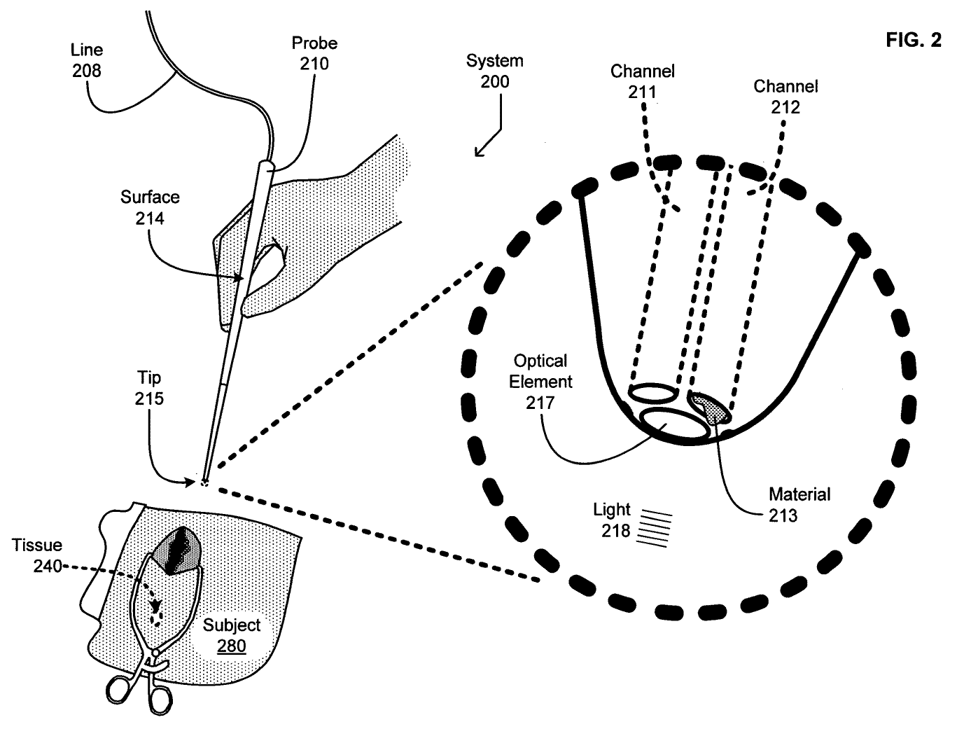

With reference now to FIG. 2, shown is a context (in a surgery or necropsy, e.g.) in which one or more technologies may be implemented. System 200 may include one or more instances of a probe 210 with a handling control surface 214 and a distal portion that can be extended into tissue 240 as shown. A magnified view of tip 215, for example, reveals a lens or other optical element 217 at least configured to receive light 218 from tissue 240. In some variants, optical element 217 includes or otherwise operates in conjunction with a laser, infrared, ultrasound, or other emitter that transmits light 218 or other energy into tissue 240. Alternatively or additionally, probe 210 may include one or more channels 211 or other chambers for receiving fluid and/or tissue samples (via a partial vacuum or other extraction element, e.g.). Probe 210 may likewise include one or more channels 211 configured to dispense stains, therapeutic agents, or other materials 213 to tissue 240 in vivo of subject 280 before or without extraction. In some variants, probe 210 (or a portion of it that includes tip 215) may be separated (from line 208, e.g.) so that a tissue sample or other extraction may be stored or transported apart from a remainder of system 200.

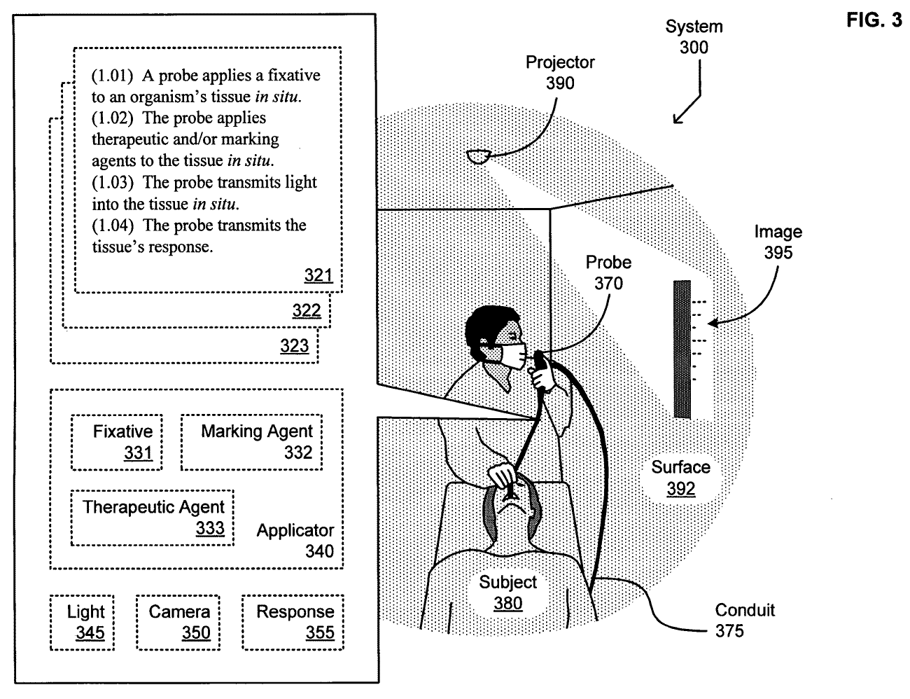

With reference now to FIG. 3, shown is a context in which one or more technologies may be implemented for performing one or more protocols 321, 322, 323. In protocol 321, for example, (1.01) a probe applies a fixative to an organism's tissue in situ; (1.02) the probe applies therapeutic and/or marking agents to the tissue in situ; (1.03) the probe transmits light into the tissue in situ; and (1.04) the probe transmits the tissue's response. As shown, system 300 may include a probe 370 having one or more applicators 340 or other dispensers configured to apply one or more fixatives 331, marking agents 332, therapeutic agents 333, or other treatment material to tissue of a human or other subject 380 in situ. Probe 370 may further include or operate in conjunction with (a) a light 345 or other optical element configured to transmit light into the tissue of subject 380 in situ and/or (b) a camera 350, conduit 375, or other output module configured to transmit an image, measurement, or other indication of the tissue's response 355 to the treatment material(s) and the light. In some contexts, for example, probe 370 may transmit one or more images 125, 395 (in an electrical or optical signal, e.g.) via conduit 375, projector 390, a projection surface 392, or other such physical media 100 as described below. In some protocols 322, for example, probe 370 may function as a bronchoscope or endoscope in a first mode (for approaching a growth of concern, for example) and as a surgical and/or histological instrument in a second mode.

In light of teachings herein, numerous existing techniques may be applied for preparing fluorescent or other marking agents as described herein without undue experimentation. See, e.g., Jim Krause, Color Index: Over 1,000 Color Combinations, CMYK and RGB Formulas, for Print and Web Media, (2002) F&W Publications, Inc., ISBN: 1581802366; Conn's Biological Stains: A Handbook of Dyes, Stains and Fluorochromes for Use in Biology and Medicine, (10^{th} Ed. 2002) Bios Scientific Pub. Ltd., ISBN: 9781859960998; U.S. Pat. No. 7,326,575 (“Methods and compositions for the preparation and use of fixed-treated cell-lines and tissue in fluorescence in situ hybridization”); U.S. Pat. No. 7,319,046 (“Integrated optoelectronic silicon biosensor for the detection of biomolecules labeled with chromophore groups or nanoparticles”); U.S. Pat. No. 6,830,743 (“In Vivo stain compounds and methods of use to identify dysplastic tissue”); U.S. Pat. No. 6,790,636 (“Rapid fluorescent labeling of tissue for microdissection using fluorescent specific binding agents”); U.S. Pat. No. 6,608,213 (“Fluorescence-labeled probe for DNA and a fluorescence-labeled plasmid”); U.S. Pat. No. 6,599,496 (“Endoscopy tissue stain”); U.S. Pat. No. 6,372,451 (“Histochemical labeling stain for myelin in brain tissue”); U.S. Pat. No. 6,333,110 (“Functionalized nanocrystals as visual tissue-specific imaging agents, and methods for fluorescence imaging”); U.S. Pat. No. 6,106,804 (“Arsenic-72 labeled compounds for tissue specific medical imaging”); U.S. Pat. No. 5,965,713 (“Dye labeled protein conjugate its preparing method and sensor using the same”). Some such variants, for example, may include media bearing one or more identifiers, components, protocols, or other indicators of optical enhancement materials and/or other useful components. Alternatively or additionally, such modules may implement or otherwise interact with one or more materials or other components for staining, for example.

In light of teachings herein, numerous existing techniques may be applied for configuring fixatives or other modes of protecting tissue or other extractions as described herein without undue experimentation. See, e.g., U.S. Pat. No. 7,374,907 (“System and method for automatically processing tissue samples”); U.S. Pat. No. 7,264,471 (“Methods and kits for bleaching teeth while protecting adjacent gingival tissue”); U.S. Pat. No. 7,229,418 (“Tissue specimen encapsulation device and method thereof”); U.S. Pat. No. 6,743,254 (“Tissue expander with protection against accidental puncture”); U.S. Pat. No. 6,673,006 (“Tissue positioning apparatus and method for protecting tissue from radiotherapy”); U.S. Pat. No. 6,640,139 (“Thermal therapy with tissue protection”); U.S. Pat. No. 6,49,4902 (“Method for creating a virtual electrode for the ablation of tissue and for selected protection of tissue during an ablation”); U.S. Pat. No. 5,843,086 (“Thermal bone cement removal system with tissue protector”); U.S. Pat. No. 7,138,226 (“Preservation of RNA and morphology in cells and tissues”); U.S. Pat. No. 6,875,583 (“Rapid microwave-assisted fixation of fresh tissue”); U.S. Pat. No. 6,586,713 (“Apparatus for high quality, continuous throughput, tissue fixation-dehydration-fat removal-impregnation”); U.S. Pat. No. 6,204,375 (“Methods and reagents for preserving RNA in cell and tissue samples”); U.S. Pat. No. 6,017,725 (“Cytological fixative and dehydrating agent for treating histological and cytological tissue”).

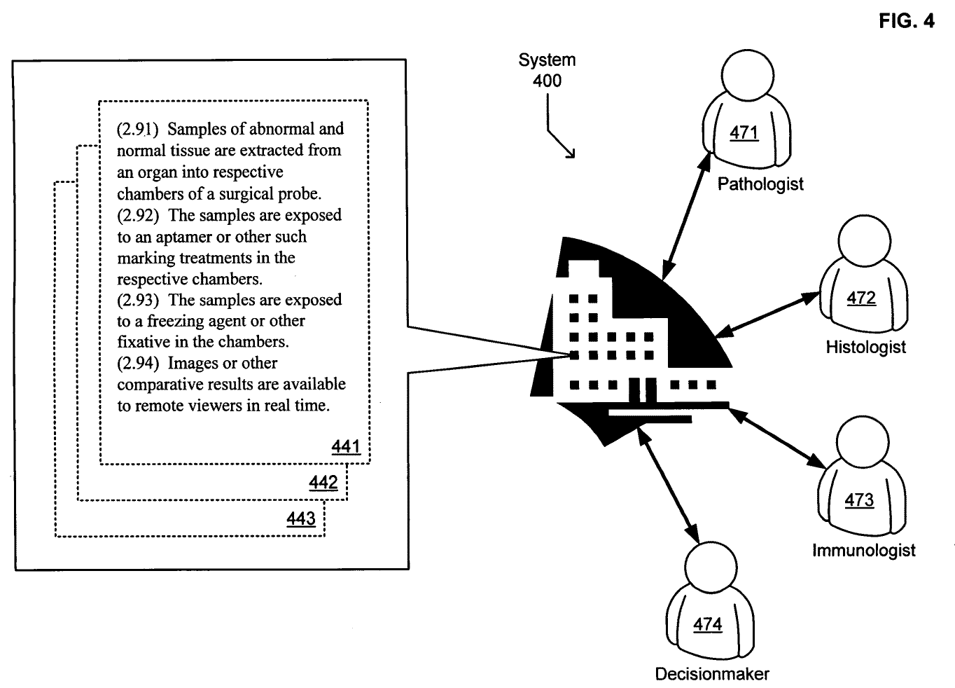

With reference now to FIG. 4, shown is a context in which one or more technologies may be implemented for performing one or more protocols 441, 442, 443. In protocol 441, for example, (2.91) samples of abnormal and normal tissue are extracted from an organ into respective chambers of a surgical probe; (2.92) the samples are exposed to an aptamer or other such marking treatments in the respective chambers; (2.93) the samples are exposed to a freezing agent or other fixative in the chambers; and (2.94) images or other comparative results are available to remote viewers in real time. In a distributed system 400, for example, such viewers or other participants may include one or more pathologists 471, histologists 472, immunologists 473, or other such experts who can provide identifiers 142 materials or protocols 442 (for tissue typing, cancer staging, marking, treatment options, etc.) that are most promising and timely, for example, in light of new information about a given subject and situation. In some contexts, for example, a pathologist 471 or histologist 472 may use a preliminary image or observation of an organism's abnormal tissue to retrieve pertinent images 124 or other reference information (from <http://health.nih.gov/>, <http://seer.cancer.gov/>, or other public or private providers, e.g.) superseding, supplementing, or obviating comparative information representing an organism's healthy tissue. Alternatively or additionally, such information may be used by specialists or other decisionmakers 474 to facilitate procedural decisions informed by medical or veterinary context in real time.

Some variants may include software-controlled or other special-purpose circuitry for sharing images, evaluation results or other device-detectable data with remote resources in real time. In light of teachings herein, numerous existing techniques may be applied for implementing communication conduits or other modules as described herein without undue experimentation. See, e.g., U.S. Pat. No. 7,36,7018 (“System and method for organizing and sharing of process plant design and operations data”); U.S. Pat. No. 7,203,625 (“Multisided sharing of dynamic data in a wireless test environment”); U.S. Pat. No. 7,177,874 (“System and method for generating and processing results data in a distributed system”); U.S. Pat. No. 7,162,476 (“System and method for sharing global data within distributed computing systems”); U.S. Pat. No. 7,119,666 (“Method for controlling and evaluating a sensor device shared by a plurality of applications”); U.S. Pat. No. 7,020,699 (“Test result analyzer in a distributed processing framework system and methods for implementing the same”); U.S. Pat. No. 6,308,175 (“Integrated collaborative/content-based filter structure employing selectively shared, content-based profile data to evaluate information entities in a massive information network”).

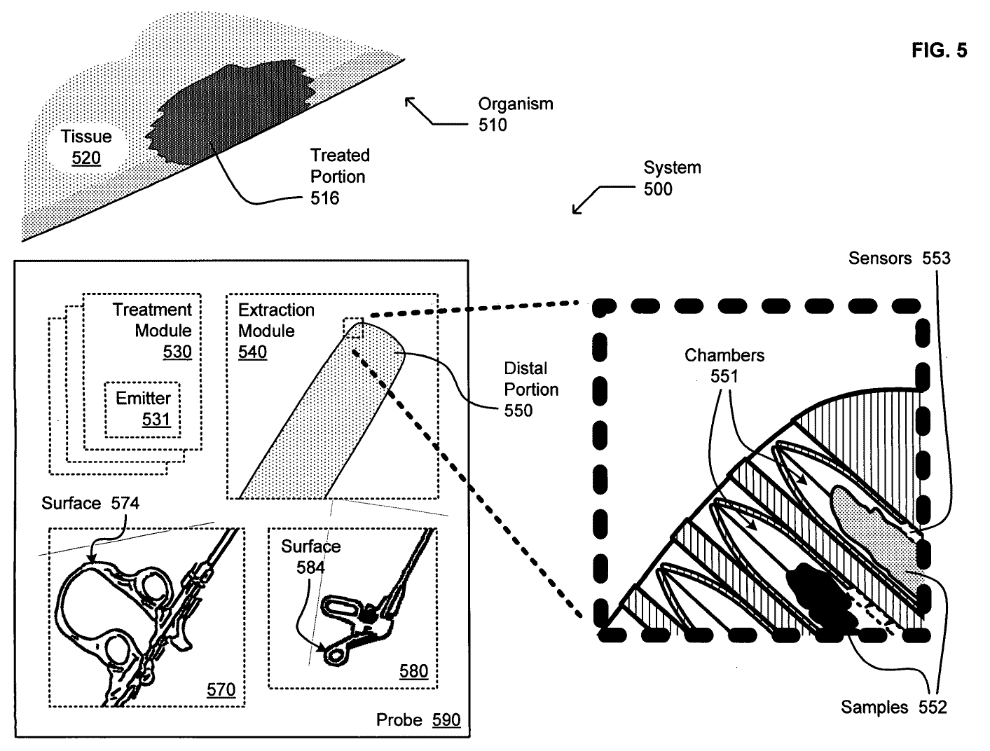

With reference now to FIG. 5, shown is a context in which system 500 is configured for accessing at least a treated portion 516 of external or other tissue 520 of an organism 510. A laparoscopic or other probe 590 includes at least an extraction module 540 (in a distal portion 550 of probe 590, e.g.) that a pathologist or surgeon can manipulate via one or more handling control surfaces 574, 584 (of any of several handle configurations 570, 580 shown herein, for example).

An embodiment provides one or more media 100 bearing device-detectable data 191 indicating a treatment of one or more tissue samples 552 in one or more chambers 551 extended into tissue 520 of an organism 510. In some variants, some such media may bear a component of the device-detectable data 191 that was generated while such chemical or other treatments were applied to the tissue sample(s) 552. In some contexts, for example, at least one of the treatment modules 530 include an emitter 531 configured to emit ultrasonic, microwave, laser, or other energy into one or more chambers 551 of the extraction module(s) 540, such as for permeabilizing, mixing, curing, severing, or otherwise treating the tissue samples 552. Alternatively or additionally, extraction module 540 may (optionally) include one or more sensors 553 or other detection circuitry beside the chamber(s), such as for controlling or detecting a result 192 of such treatments.

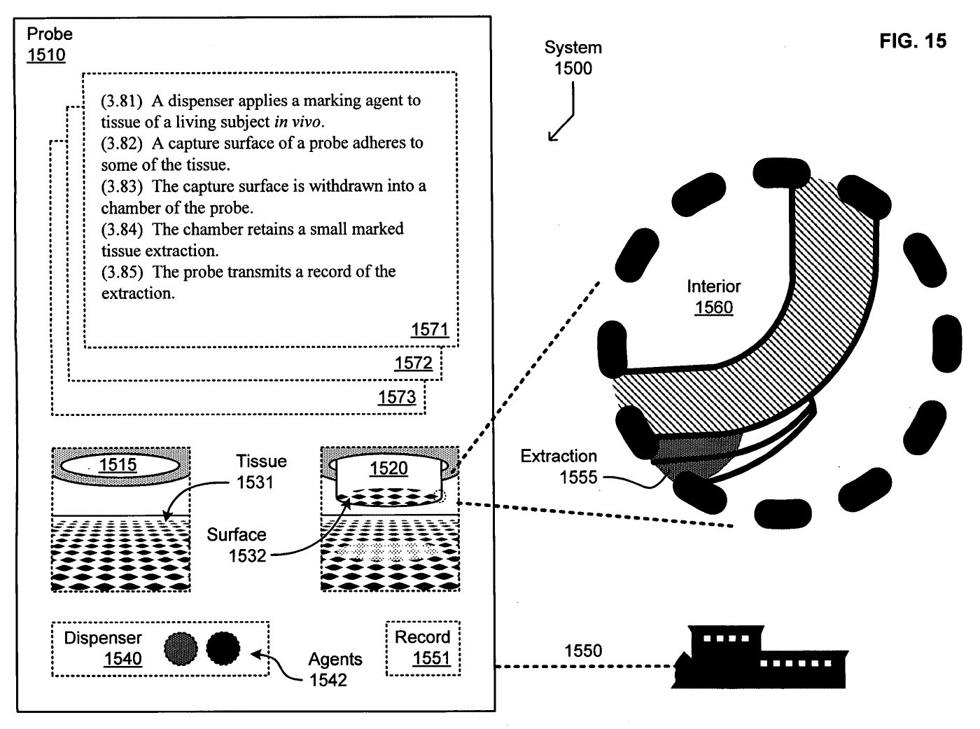

An embodiment provides one or more physical media 100, 1000 bearing one or more images 395 or other device-detectable data indicating an extraction of (at least some of a chemically) treated portion 516 of tissue 520 frozen in vivo. This can occur, for example, in a context in which tissue 520 includes a portion of a mucous membrane of subject 380 treated via applicator 340, imaged, and then extracted by a freezing capture surface. See FIG. 15. In some variants, for example, a text component of image 395 can include an identifier or other descriptor of one or more protocols 114, 115 by which this was performed.

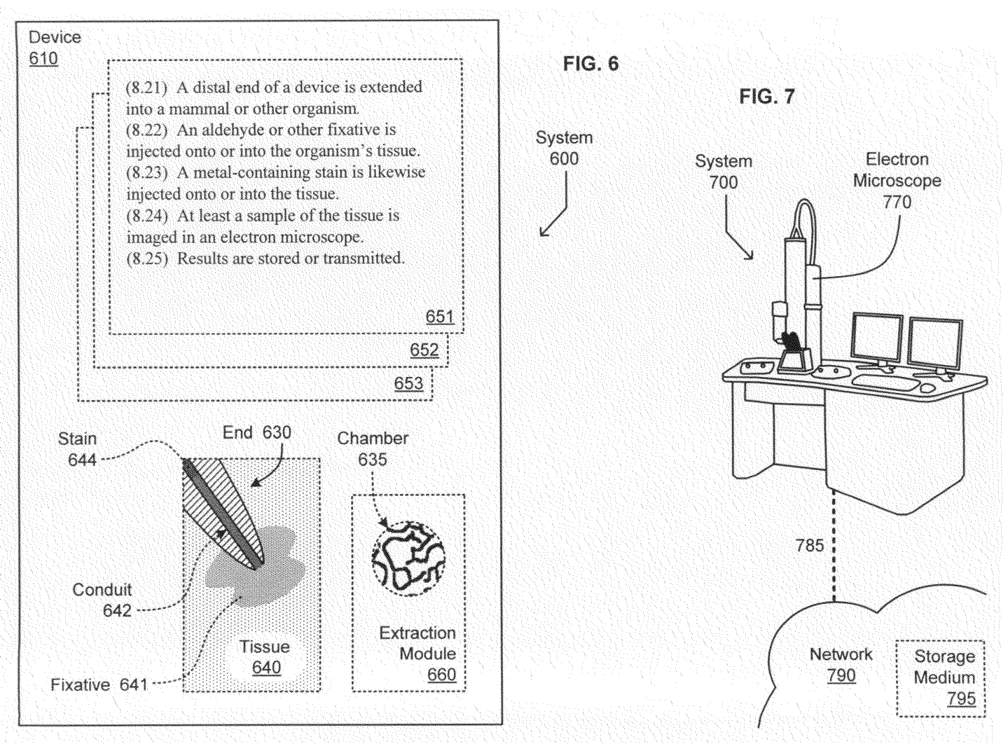

With reference now to FIG. 6, shown is a context in which one or more technologies may be implemented for performing one or more protocols 651, 652, 653. In protocol 651, for example, (8.21) a distal end of a device is extended into a mammal or other subject; (8.22) an aldehyde or other fixative is injected onto or into the organism's tissue; (8.23) a metal-containing stain is likewise injected onto or into the tissue; (8.24) at least a sample of the tissue is imaged in an electron microscope; and (8.25) results are stored or transmitted. System 600 may include a syringe or other device 610, for example, configured to permit end 630 inject a fixative 641, label, and/or other material into or onto tissue 640 (via conduit 642, e.g.). Device 610 may likewise be configured to inject stain 644 (a uranium- or lead-containing stain, e.g.) and/or other materials (seconds or minutes later, e.g.) into or onto an overlapping region of tissue 640.

An embodiment provides one or more media 100 bearing a device-detectable image 123 of tissue 640 (in or from subject 280, e.g.) to which a stain 644 or other optical enhancement material has been applied in vivo. In some contexts, for example, a sample of tissue 640 can be received into a chamber 635 of extraction module 660 a few seconds or minutes before the image is generated. In some variants, a “device-detectable image” of one or more cells may include one in which a contiguous grouping of many pixels graphically depict (a) a cell's relationship to one or more neighboring cells or (b) some other useful morphological indication of at least one cell. Many tumors can be characterized effectively by providing a small image of several nuclei in a group, for example, even if cell boundaries are not readily apparent.

An embodiment provides one or more physical media 100 bearing a measurement or other device-detectable data indicating a fixative 641 or other treatment of a tissue component in one or more chambers 551, 635 that have been extended into tissue 240, 520 of an organism 510. This can occur, for example, in a context in which treated portion 516 overlaps tissue 640 and in which at least some such treatment occurs in the chamber(s). In some variants, for example, the above-described systems and methods may generate or otherwise operate in conjunction with device-detectable data generated (a) while or after the chamber was extended into an organism's tissue and/or (b) while or after an optical enhancement material or other treatment was applied to a sample of the organism's tissue. Alternatively or additionally, such embodiments may include a context in which a microtome is configured to extract the tissue sample by severing one or more portions of the tissue in the chamber from a remainder of an extracted structure.

Alternatively or additionally, such media 100 may bear one or more indicators 161, 162 at least suggesting a yes/no protocol decision about various tissues 240, 520, 640 having at least one treated portion 516 to which a stain 644 or other optical enhancement material was applied in vivo. This can occur, for example, in a context in which a surgeon selects and/or designs a protocol for deciding whether to extract abnormal tissues.

With reference now to FIG. 7, shown is a system 700 in which one or more technologies may be implemented for completing one or more protocols 651, 652. In some variants, an extraction module 660 or entirety of device 610 may be observed via electron microscope 770, which can then transmit such images or other data via a conduit 785 or other signal-bearing medium. Alternatively or additionally, such data may be retained in a storage medium 795 or other data-handling device (of network 790, e.g.).

An embodiment provides (a) a conduit 785 or storage medium 795 bearing a device-detectable image 123 of cells to which an optical enhancement material was applied in vivo and (b) an extraction module 660 configured to contain the cells. This can occur, for example, in a context in which system 700 includes or otherwise interacts with at least an extraction module 660 of device 610. Alternatively or additionally, the storage or transmission media 100 may indicate one or more therapeutic and/or timing protocols 115, such as an indication of a chemotherapy or other regimen that may have affected the tissue 640 in the minutes or days before extraction. In some transmission electron microscopy (TEM) protocols, moreover, an ultramicrotome may be used for sectioning a tissue sample (to about 100 nanometers or less, e.g.) embedded in epoxy resin within chamber 635.

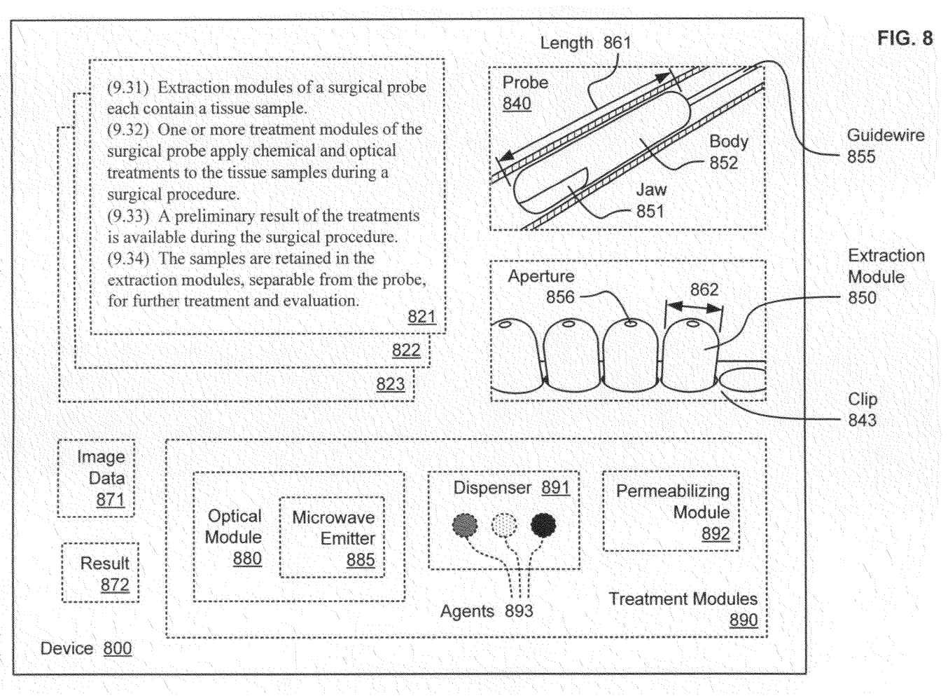

With reference now to FIG. 8, shown is a context in which one or more technologies may be implemented for performing one or more protocols 821, 822, 823.

In protocol 821, for example, (9.31) extraction modules of a surgical probe each contain a tissue sample; (9.32) one or more treatment modules of the surgical probe apply chemical and optical treatments to the tissue samples during a surgical procedure; (9.33) a preliminary result of the treatments is available during the surgical procedure; and (9.34) the samples are retained in the extraction modules, separable from the probe, for further treatment and evaluation. Device 800 may include a laparascopic or other elongated probe 840, for example, by which one or more extraction modules 850 can each be moved quickly into position (adjacent an organism's tissue, e.g.) by a guidewire 855 or pneumatic conveying system. In some variants, for example, device 800 can contain several extraction modules each have a length 861 less than a centimeter and a width 862 of about a millimeter or less. Alternatively or additionally, each extraction module 850 may include a hollow body 852 and one or more jaws 851 that can open to permit a tissue extraction. In some variants, each may also have one or more apertures 856 (a) for engaging a threaded or other guidewire, (b) for receiving treatment material before or after extraction, (c) for sterilizing or otherwise preparing a containment chamber for the extraction, (d) for delivering energy into a sample as a mode of treatment, (e) for depositing a solvent as a mode of treatment, (f) for drawing a vacuum so that tissue enters the chamber, (g) to permit a sensor or other detection circuitry to access a tissue sample, and/or (h) for other purposes as described herein. Device 800 may likewise include a clip 843 or other such structure configured to hold several extraction modules 850 before and/or after extraction.

In some variants, for example, device 800 may include one or more instances of microwave emitters 885 or other optical modules 880, various agents 893 that can be applied or accessed (via a dispenser 891, e.g.) as described herein, permeabilizing modules 892, or other such treatment modules 890 operable for use with an extraction 20 module or other chamber as described herein. This can occur, for example, in a context in which one or more dispensers or other permeabilizing modules 892 operable for chemically, thermally, temporarily, mechanically, or otherwise permeabilizing an organic membrane to facilitate various treatments as described herein without undue experimentation. See, e.g., U.S. Pat. No. 7,412,284 (“Apparatus for electroporation mediated delivery for drugs and genes”); U.S. Pat. No. 7,393,680 (“Combined electroporation and microinjection method for the penetration of lipid bilayer membranes”); U.S. Pat. No. 7,306,940 (“Electroporation device and method, delivering a modulated signal under continuous control of cell electropermeabilization”); U.S. Pat. No. 7,271,005 (“Modulation of bacterial membrane permeability”); U.S. Pat. No. 7,186,559 (“Apparatus and method for electroporation of biological samples”); U.S. Pat. No. 6,846,668 (“Microfabricated cell injector”); U.S. Pat. No. 6,706,088 (“Method for controlling membrane permeability by microwave and method for producing organic separation membrane”); U.S. Pat. No. 6,589,503 (“Membrane-permeant peptide complexes for medical imaging, diagnostics, and pharmaceutical therapy”); U.S. Pat. No. 6,319,901 (“Methods for prolonging cell membrane permeability”); U.S. Pat. No. 6,015,834 (“In vivo treatment of mammalian cells with a cell membrane permeant calcium buffer”).

An embodiment provides (a) a probe 370, 590, 840 having one or more extraction modules 540, 850; (b) a treatment module 530, 890 configured to apply material or other treatment to tissue 520, 640 in the extraction module(s); and (c) one or more sensors or other output modules configured to transmit one or more measurements 153, image data 871, or other results 192 of such treatment from the probe via an antenna or other physical medium.

An embodiment provides a probe 590, 840 or other device 800 comprising (a) a handling control surface 574, 584 (b) one or more distal portions 550, narrow enough to extend into a living organism 510, (c) a first dispenser 891 configured to apply a marking agent 332 or other treatment material(s) to tissue 240, 520 adjacent the device, and (d) one or more instances of sensors 553, equipment, or other output modules configured to transmit an image or other result 192 of the treatment (via one or more conduits 375, 785, e.g.). This can occur, for example, in a context in which device 800 combines features of several of the probes 210, 370 590, 840 as described herein configured, for example, to transmit the result through line 208.

Alternatively or additionally, such devices 800 may comprise a dispenser 891 configured to apply a marking agent 332 or other treatment material(s) to tissue 240, 520 of an organism 510 in vivo, an emitter 531 or other optical element 217 configured to transmit light 218 into the tissue of the organism in vivo, and one or more instances of sensors 553, imaging or measurement equipment, or other output modules configured to transmit a result 192 of at least the light and the treatment material(s) upon the tissue of the organism in vivo. This can occur, for example, in a context in which device 800 combines features of several of the probes 210, 370 590, 840 as described herein configured, for example, to transmit the result to or through medium 100.

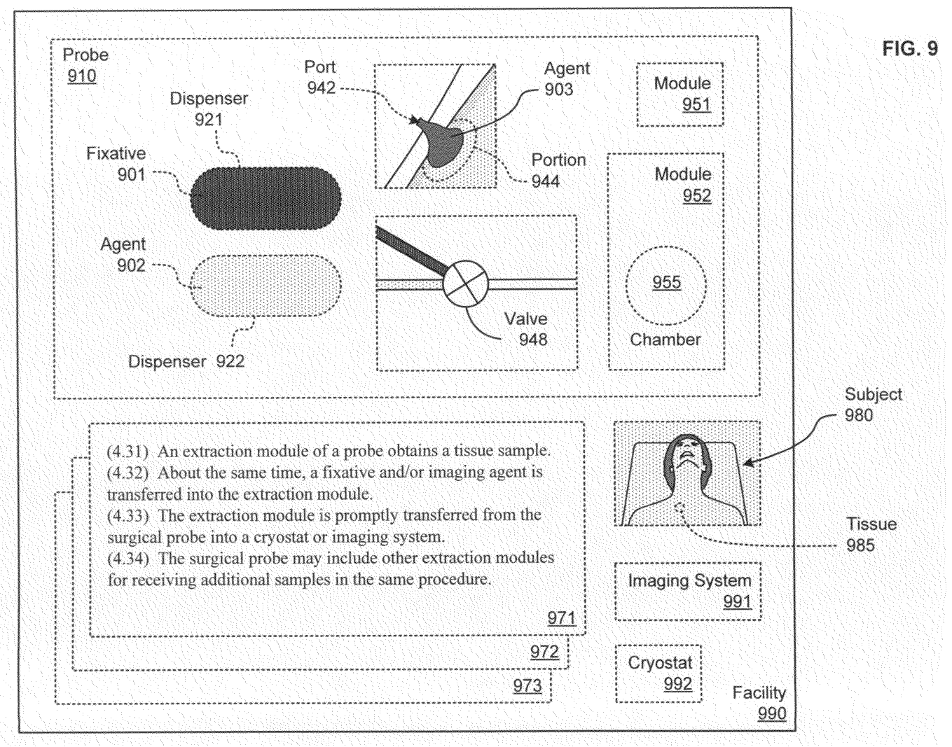

With reference now to FIG. 9, shown is a facility 990 in which one or more technologies may be implemented for performing one or more protocols 971, 972, 973. In protocol 971, for example, (4.31) an extraction module of a probe obtains a tissue sample; (4.32) about the same time, a fixative and/or imaging agent is transferred into the extraction module; (4.33) the extraction module is promptly transferred from the surgical probe into a cryostat or imaging system; and (4.34) the surgical probe may include other extraction modules for receiving additional samples in the same procedure. Some instances of probe 910 may include one or more (a) dispensers 921 configured to administer a compound or other fixative 901 or (b) dispensers 922 configured to administer another agent 902 as described herein. In some contexts, for example, probe 910 may include a port 942 configured to inject a gel or other liquid-containing agent 903 onto a portion 944 of tissue 985 of a subject 980. Alternatively or additionally, probe 910 may include a mixing or other control valve 948 effective for dispensing one or more just-mixed materials into a port, for example, or one or more chambers 955 of an extraction module 952. In some protocols 972, probe 910 may be configured to perform such dispensations within a few minutes or seconds of an extraction. Alternatively or additionally, one or more extraction modules 951, 952 may be transferred promptly after extraction from probe 910 into an ultrasound or other imaging system 991 or into a cryostat 992.

An embodiment provides one or more media 100 bearing device-detectable data 191 depicting, characterizing, or otherwise indicating an extraction of chemically treated tissue frozen in vivo, such as by injecting a freezing agent (as agent 902 or agent 903, e.g.) onto a portion 944 of tissue 985 that has been stained or otherwise treated with an optical enhancement material.

Another embodiment provides a probe 590, 910 or other device comprising one or more handling control surfaces 574, 584; a distal portion 550 narrow enough to protrude into living tissue 520; a first dispenser 921 configured to apply a compound or other agent 902 to treat tissue 520 adjacent the distal portion 550 as described herein; and one or more sensors 553 or other output modules configured to transmit one or more results 192, 193 of the agent 902. This can occur, for example, in an embodiment that combines features of probe 590 and probe 910 in a device operably coupled with medium 100. In some variants, moreover, such results 192 can likewise depend upon artificial illumination (from emitter 531, e.g.), chemical treatments (in chamber 955, e.g.), or other protocol features as described herein.

Such a compound may, in some variants, include an aldehyde or other cross-linking fixative as described herein. See, e.g., U.S. Pat. No. 7,07,5045 (“Automatic, microwave assisted tissue histoprocessor”); U.S. Pat. No. 6,875,583 (“Rapid microwave-assisted fixation of fresh tissue”); U.S. Pat. No. 6,319,683 (“Method and composition for controlling formaldehyde fixation by delayed quenching”); U.S. Pat. No. 6,296,608 (“Diagnosing and performing interventional procedures on tissue in vivo”); U.S. Pat. No. 6,008,292 (“Method for inhibiting calcification of aldehyde-fixed bioprosthetic materials”).

Alternatively or additionally, such materials may include an artificial fluorescent or other luminescent marking agent, such as may be administered via ports 942 or other dispensers 891, 922 operable to mark (some or all of) the tissue sample. In light of teachings herein, numerous existing techniques may be applied for implementing and dispensing such materials as described herein without undue experimentation. See, e.g., U.S. Pat. No. 7,414,117 (“Nucleotide derivative and DNA microarray”); U.S. Pat. No. 7,378,245 (“Methods for detecting and localizing DNA mutations by microarray”); U.S. Pat. No. 7,155,050 (“Method of analyzing cell samples, by creating and analyzing a resultant image”); U.S. Pat. No. 7,153,691 (“Method of identifying and assessing DNA euchromatin in biological cells for detecting disease, monitoring wellness, assessing bio-activity, and screening pharmacological agents”); U.S. Pat. No. 7,129,344 (“Nucleic acid isolation”); U.S. Pat. No. 6,924,373 (“DNA labeling reagents, acridinium-9-carboxamide derivatives and process of preparing DNA labeling compounds”); U.S. Pat. No. 6,830,889 (“Method of detecting DNA by DNA hybridization method with the use of fluorescent resonance energy transfer”); U.S. Pat. No. 6,716,394 (“DNA sequencing using multiple fluorescent labels being distinguishable by their decay times”); U.S. Pat. No. 6,608,213 (“Fluorescence-labeled probe for DNA and a fluorescence-labeled plasmid”); U.S. Pat. No. 6,428,667 (“Multichromophore fluorescent probes using DNA intercalation complexes”); U.S. Pat. No. 6,346,379 (“Thermostable DNA polymerases incorporating nucleoside triphosphates labeled with fluorescein family dyes”); U.S. Pat. No. 5,942,410 (“Composition and method for staining cellular DNA, comprising thiazine derivative metabisulfite and methanol or ethanol”).

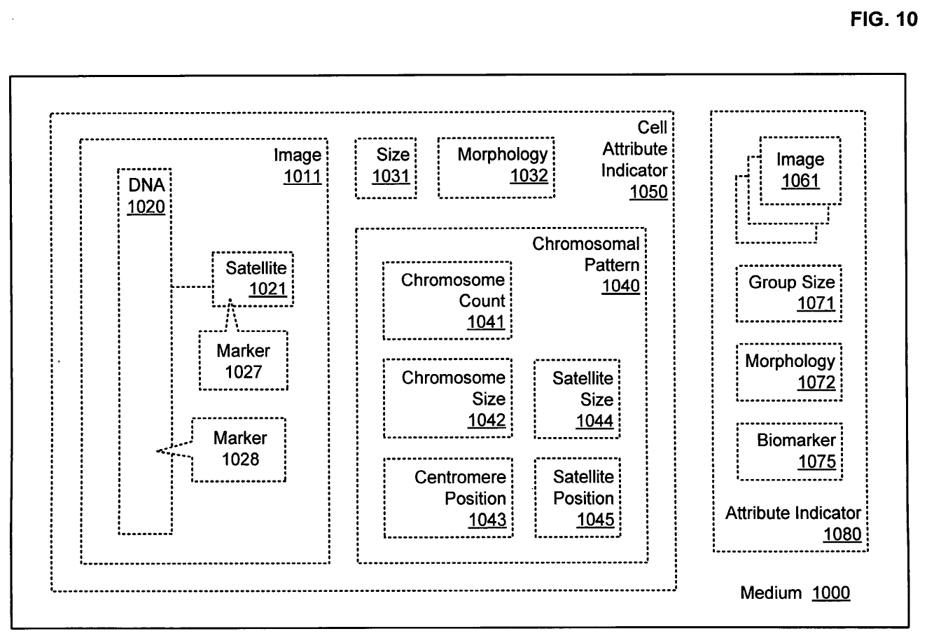

With reference now to FIG. 10, shown is a medical or veterinary system in which one or more technologies may be implemented. As described below, it includes one or more media 1000 (configured for storage or presentation, e.g.) bearing one or more instances of cell attribute indicators 1050 or other attribute indicators 1080. Such cell attribute indicators can include one or more instances of images 1011, optionally relating to DNA 1020 or other such large molecules, satellite DNA 1021 or other such polyatomic fragments, or to one or more markers 1027, 1028 that may attach at specific locations on some such molecules or fragments that may be present within a cell. Southern blots, northern blots, western blots, microarray analysis, in situ hybridization, and many other existing protocols permit cell characterizations using such markers observable by autoradiography, spectrophotometry, densitometry, chromatography, or other such modes of detection as described herein. Alternatively or additionally, cells or cell features may likewise be characterized by their sizes 1031 or morphologies 1032. Chromosomal patterns 1040, for example, may be characterized by one or more chromosome counts 1041, chromosome sizes 1042, centromere positions 1043, satellite sizes 1044, satellite positions 1045, or other such observable features.

Other attribute indicators 1080 may relate to tissue or other extractions as described herein. Such indicators may include comparative or other images 1061, cell group sizes 1071, morphologies 1072, or biomarkers 1075 observable in sputum, thinly sliced tissue samples, or various other extractions as described herein.

With reference now to FIG. 11, shown is a context in which one or more technologies may be implemented. System 1100 may include one or more instances of an instrument 1110 operable to transmit respective signals 1135 to one or more evaluation modules 1140, such as via a conduit 1130 or other mode of network connection. A variety of protocols 1121, 1122, 1123 as described herein are provided for permitting one or more cartridges 1101 containing reagents 1111 to interact with one or more cartridges 1102 or other extraction modules containing tissue samples 1112, analytes, or other detectable cell or tissue features of interest.

Such evaluation modules may (optionally) reside remotely from instrument 1110 and/or operate roughly contemporaneously with protocols 1121, 1122 or even within some protocols 1122 applied by instrument 1110. Such protocols may invoke one or more type recognition modules 1151, image recognition modules 1152, or other modules 1153, 1154, 1155, 1156, 1157, 1158, 1159 of pattern recognition logic 1150. Such logic may, as exemplified below, trigger an application of and characterization by one or more thresholds 1171 or other criteria 1172 of one or more profiles 1181, 1182, 1183, 1184, 1185 of evaluation data 1180 specified by a pathologist or other expert, for example, such as those depicted in FIG. 4. Alternatively or additionally, such experts may provide, apply, or otherwise interact with one or more images 1191, types 1192, values 1193, results 1194, or other work product as described with reference to FIG. 1. In some contexts, for example, a service provider may keep such image processing, personal knowledge, or other evaluation tools as trade secrets, even while conveying recommendations 144 or other results to facilities at which such instruments reside. Product providers may likewise supply cartridges 1101 with proprietary formulations of reagents 1111, for example, to foster refinements in reagent formulation and other tissue characterization protocols as described herein.

An embodiment provides (a) a dispenser 891 configured to apply a marking agent 332 or other treatment material(s) to tissue 240, 520 of an organism 510 in vivo; (b) an agent 903 or other cooling component configured to freeze at least some of the tissue in vivo; and (c) a cartridge 1102, laser, or other such extraction element configured to remove at least a sample 1112 of the tissue 240, 520 from the organism. This can occur, for example, in an implementation combining features of several of the probes 210, 370 590, 840, 910 as described herein configured, for example, to extract the sample into chamber 955.

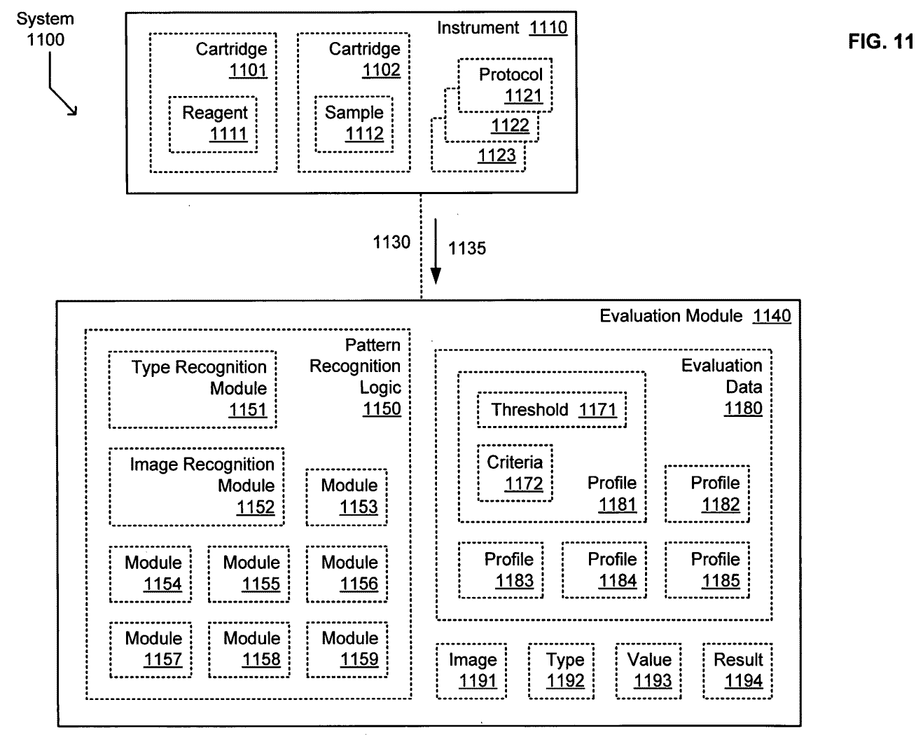

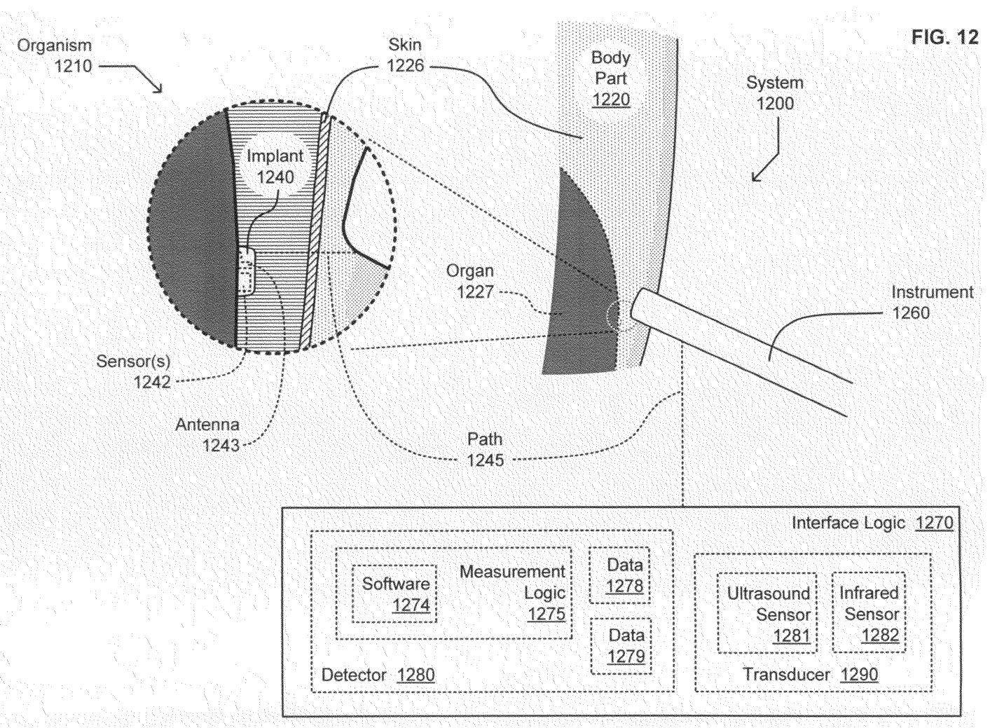

With reference now to FIG. 12, shown is a system 1200 in which one or more technologies may be implemented, such as for one or more body parts 1220 of subject 1210 to interact with interface logic 1270 via one or more instruments 1260 (manipulable via one or more handling control surfaces, e.g.). As shown, body part 1220 contains one or more chips or other implants 1240 positioned under the organism's skin 1226 in tissue adjacent organ 1227. Implant 1240 may (optionally) include one or more sensors 1242 as described below and/or one or more antennas 1243 operable for receiving and/or transmitting data along wireless data path 1245 as shown. Interface logic 1270 may include one or more instances of detectors 1280 and/or transducers 1290 such as ultrasound sensors 1281 or infrared sensors 1282. Alternatively or additionally, detector 1280 may include special-purpose software 1274 or other such measurement logic 1275 configured to handle configuration, control, measurement, or other data 1278, 1279 as described below.

An embodiment provides an instrument 1260 having at least (a) a chamber 551, 635, 955, or other cavity in which one or more sample treatment protocols 443 may be applied to a tissue sample 552, and (b) interface logic 1290, sensors, or other such output modules configured to transmit one or more measurements 153, images, or other results 192 of such treatment. In some variants, for example, the instrument may include or otherwise interact with a treatment module 530 configured to apply one or more fixatives 331, types of light 218 or other energy, marking agents 332 or other treatments.

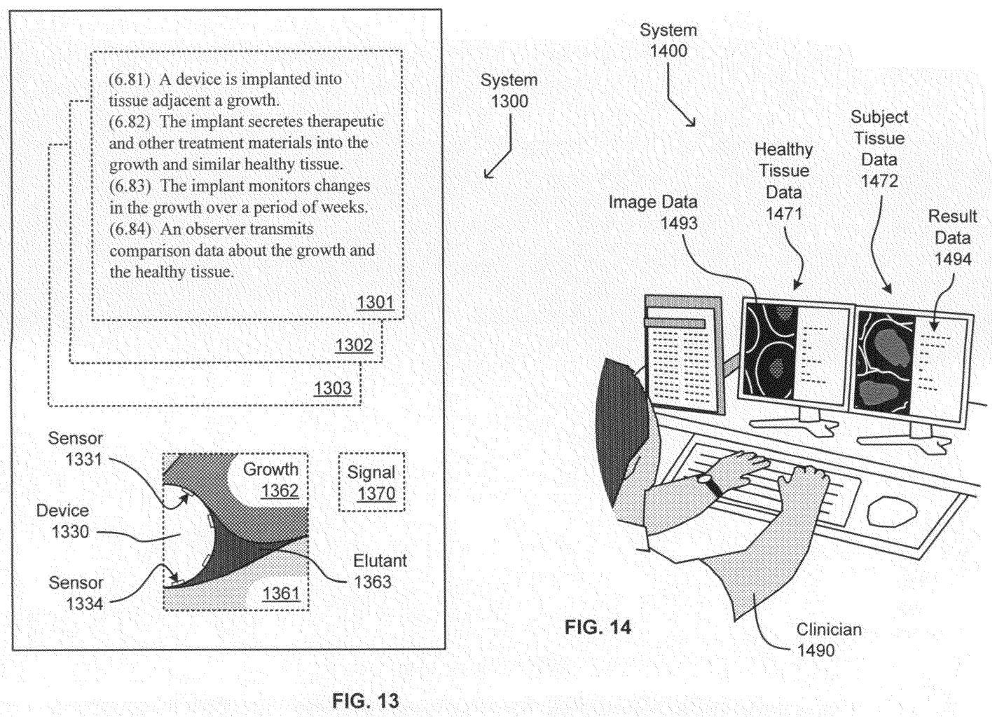

With reference now to FIG. 13, shown is a context in which one or more technologies may be implemented for performing one or more protocols 1301, 1302, 1303. In protocol 1301, for example, (6.81) a device is implanted into tissue adjacent a growth; (6.82) the implant secretes therapeutic and other treatment materials into the growth and similar healthy tissue; (6.83) the implant monitors changes in the growth over a period of weeks; and (6.84) an observer transmits comparison data about the growth and the healthy tissue. In system 1300, for example, an implant or other such device 1330 may be positioned adjacent healthy tissue 1361 and/or a growth 1362. In some protocols 1301, 1302, for example, device 1330 may be configured to dispense one or more optical enhancement materials, elutants 1363, or therapeutic material to one or more tissues in vivo. Alternatively or additionally, device 1330 may include one or more sensors 1331, 1334 or other detection circuitry for transmitting signals 1370 about such tissues in vivo.

With reference now to FIG. 14, shown is a system 1400 in which one or more technologies may be implemented, optionally for use in conjunction with any of FIGS. 1-13. As shown, a clinician 1490 or other observer is able to compare image data 1493 or other result data 1494 depicting several cells (of healthy tissue 1361, e.g.) with another image of several cells (of growth 1362, e.g.).

An embodiment provides a display, conduit, memory, or other physical medium 100, 1000 bearing healthy tissue data 1471, subject tissue data 1472, or other data containing images 1061 at least partly depicting one or more cells to which a fluorescent antibody or other optical enhancement material has been applied in vivo. This can occur, for example, in a context in which one or more conduits directly or indirectly bear signal 1370 from device 1330 to system 1400. In some contexts, system 1400 may further include or otherwise interact with one or more cartridges 1102 or other extraction modules 540, 850 configured to contain cells to which an optical enhancement or other material was applied in vivo. Alternatively or additionally, system 1400 may likewise interact with (a) one or more cartridges 1101 or other dispensers of such materials and/or (b) sensors or other circuitry for transmitting such images suitable for display.

With reference now to FIG. 15, shown is a context in which one or more technologies may be implemented for performing one or more protocols 1571, 1572, 1573. In protocol 1571, for example, (3.81) a dispenser applies a marking agent to tissue of a living subject in vivo; (3.82) a capture surface of a probe adheres to some of the tissue; (3.83) the capture surface is withdrawn into a chamber of the probe; (3.84) the chamber retains a small marked tissue extraction; (3.85) the probe transmits a record of the extraction. In system 1500, for example, dispenser 1540 may (optionally) be configured to spray, inject, print, or otherwise apply a fluor or other selection of agents 1542 onto a mucous membrane or other tissue 1531 in vivo. In some variant protocols 1572 or configurations of probe 1510, tissue 1531 may be brought into contact with and then partly drawn in vivo into chamber 1515 (by a partial vacuum, e.g.) and into contact with an adhesive-coated or other capture surface 1532. In others, capture surface 1532 may retract into chamber 1515 after protruding into tissue 1531 to obtain the extraction 1555. This may occur, for example, in a context in which a freezing agent (at −10° C. or colder, e.g.) flows through interior 1560 of protrusion 1520. In some variants, for example, protrusion 1520 may (optionally) be made of a pliable material so that it flips inside out (by a partial vacuum in interior 1560, for example) so that surface 1532 becomes an upper boundary of chamber 1515 containing extraction 1555. Alternatively or additionally, probe 1510 may transmit a record 1551 or other indication of the extraction(s), such as via a conduit 1550 or other signal path. Some variants may, for example, incorporate or otherwise operate in conjunction with one or more protrusions 1520, freezing agents, or other elements configured to freeze a part in vivo (a superficial portion of a mucous membrane or plant, e.g.) as a fixative or otherwise to facilitate extraction. In some variants, liquid nitrogen or other such tissue-freezing agents may be injected along interior 1560, for example, chilling surface 1532 more than enough to adhere to tissue 1531. Alternatively or additionally, in some variants, one or more physical media 3290 may bear a spoken or other or other device-detectable data indicating a time of, an occurrence of, a protocol of, or other features of an extraction of chemically treated tissue 985, 1531 frozen in vivo.

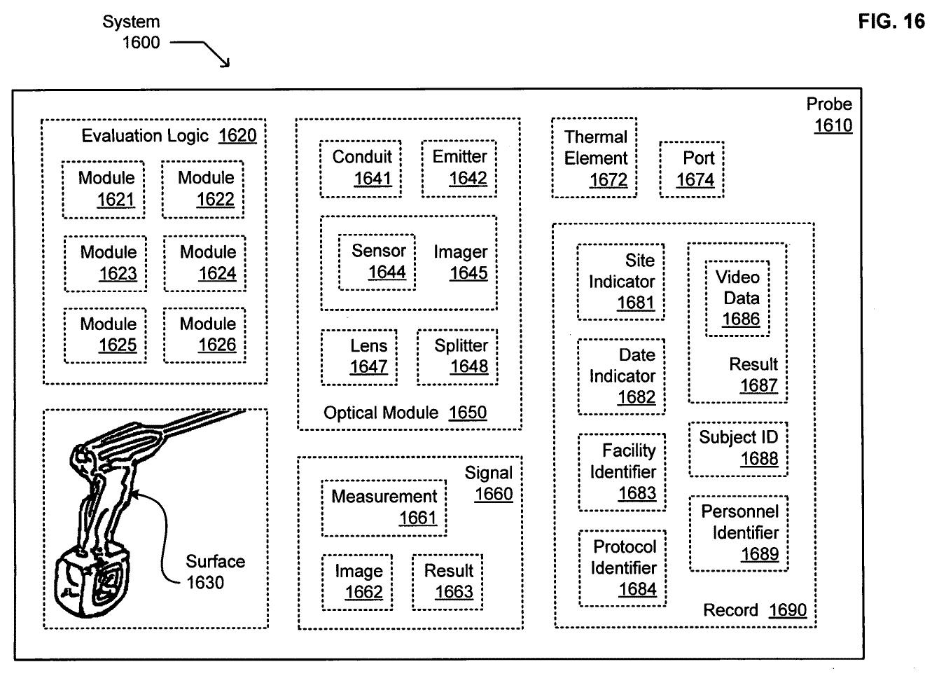

With reference now to FIG. 16, shown is a context in which one or more technologies may be implemented. System 1600 includes at least one probe 1610 configured to include or otherwise handle one or more combinations of modules 1621, 1622, 1623, 1624, 1625, 1626 of evaluation logic 1620; handling control surfaces 1630; optical modules 1650; measurements 1661, images 1662, results 1663, or other components of signal 1660; thermal elements 1672; ports 1674; or records 1690. In some variants, optical module 1650 may include one or more instances of conduits 1641, emitters 1642, sensors 1644 or other imagers 1645, lenses 1647, or optical or other signal splitters 1648. Record 1690 may include one or more instances of site indicators 1681, data indicators 1682, facility indicators 1683, protocol identifiers 1684, video data 1686 or other results 1687, subject identifiers 1688, personnel identifiers 1689, authentications, authorizations, or other such data components.

Some variants include an optical or other component configured to receive energy from a region containing a cell or other structure. Such matter or energy “from a region” may include an emission originating from the region and/or passing through the region, such as may be detected in a transmission electron microscope (TEM) or other such instruments.

An embodiment provides one or more channels 212 or other dispensers 891, 921, 922, 1540 configured to apply therapeutic and/or marking agents 1542 or other treatment materials to tissue 1531 of an organism in vivo; a cooling component (an agent 903 or capture surface 1532, e.g.) configured to freeze at least some of the tissue 1531 in vivo; and a protrusion 1520 or other extraction element configured to remove a thin extraction 1555 of the tissue 1531 from the organism. In some protocols 1571, 1573, such extractions are retained in one or more chambers 551, 955, 1515 of a probe 590, 910, 1510. Alternatively or additionally, such probes 590, 910, 1510 may be configured to transmit one or more records 1551, 1690 indicative of extraction (by conduits 1550, linkage modules 1600, or other media 100, 1000, e.g.) to an evaluation module 1140 or other resource as described herein. This can occur in a context in which system 1500 incorporates one or more features of probe 1610 as shown, for example.

Some variants may include a medium 100, 1000 bearing an indication of functional or other attributes of lipids, proteins, or other macromolecules in a sample or region. In light of teachings herein, numerous existing techniques may be applied for relating such output from one or more modules 1621 of interface or evaluation logic 1620 to a cell, organ, pathology, source, protocol, extraction, or other aspect of tissue as described herein without undue experimentation. See, e.g., U.S. Pat. No. 7,411,672 (“Method and apparatus for chemical imaging in a microfluidic circuit”); U.S. Pat. No. 7,258,775 (“Method and device for the qualitative and/or quantitative analysis of a protein and/or peptide pattern of a liquid sample that is derived from the human or animal body”); U.S. Pat. No. 7,241,578 (“Immunoassay method/equipment, biological component measurable toilet, anti-albumin monoclonal antibody, cell strain producing the same, and albumin detection kit”); U.S. Pat. No. 7,063,946 (“Methods, reagents, kits and apparatus for protein function analysis”); U.S. Pat. No. 7,005,423 (“Characterization of gene function using double stranded RNA inhibition”); U.S. Pat. No. 6,868,285 (“Method and device for detecting substances in body fluids by Raman spectroscopy”); U.S. Pat. No. 6,852,544 (“Rapid quantitative analysis of proteins or protein function in complex mixtures”); U.S. Pat. No. 6,696,271 (“Frozen tissue microarray technology for analysis of RNA, DNA, and proteins”); U.S. Pat. No. 6,410,243 (“Chromosome-wide analysis of protein-DNA interactions”); U.S. Pat. No. 6,389,306 (“Method for determining lipid and protein content of tissue”); U.S. Pat. No. 6,127,133 (“Automated analysis equipment and assay method for detecting cell surface protein function using same”); U.S. Pat. No. 6,030,768 (“Analysis of conformational changes in band 3 protein as a method for diagnosing Alzheimer's disease”).

In light of teachings herein, numerous existing techniques may likewise be applied for causing one or more chromosomal sections to be marked appropriately in response to a pathological indication as described herein. See, e.g., U.S. Pat. No. 7,176,345 (“Transgenic animals expressing light-emitting fusion proteins and diagnostic and therapeutic methods therefor”); U.S. Pat. No. 7,155,050 (“Method of analyzing cell samples, by creating and analyzing a resultant image”); U.S. Pat. No. 7,115,709 (“Methods of staining target chromosomal DNA employing high complexity nucleic acid probes”); U.S. Pat. No. 7,011,942 (“Fluorescent probes for chromosomal painting”); U.S. Pat. No. 6,975,899 (“Multi-modal optical tissue diagnostic system”); U.S. Pat. No. 6,872,817 (“Method of staining target interphase chromosomal DNA”); U.S. Pat. No. 6,607,877 (“Methods and compositions for chromosome-specific staining”); U.S. Pat. No. 6,500,612 (“Methods and compositions for chromosome 21-specific staining”); U.S. Pat. No. 6,475,720 (“Chromosome-specific staining to detect genetic rearrangements associated with chromosome 3 and/or chromosome 17”); U.S. Pat. No. 6,132,961 (“Methods of biological dosimetry employing chromosome-specific staining”); U.S. Pat. No. 5,418,169 (“Chromosome characterization using single fluorescent dye”).

Some variants may include sensors, chambers, special-purpose circuitry, or other such features configured to permit handling and observation of tissue samples 552, 1112 or other forms of matter. In light of teachings herein, numerous existing techniques may be applied for implementing one or more modules 1623 of evaluation logic 1620 for such functions as described herein without undue experimentation. See, e.g., U.S. Pat. No. 7,411,672 (“Method and apparatus for chemical imaging in a microfluidic circuit”); U.S. Pat. No. 7,308,295 (“Compilation of image information and mammography apparatus for performing biopsy”); U.S. Pat. No. 7,227,630 (“Imaging of surgical biopsies”); U.S. Pat. No. 7,149,566 (“Soft tissue orientation and imaging guide systems and methods”); U.S. Pat. No. 6,839,586 (“Use of multiphoton excitation through optical fibers for fluorescence spectroscopy in conjunction with optical biopsy needles and endoscopes”); U.S. Pat. No. 6,612,991 (“Video-assistance for ultrasound guided needle biopsy”); U.S. Pat. No. 6,500,114 (“Method of extracting biopsy cells from the breast”); U.S. Pat. No. 6,421,454 (“Optical correlator assisted detection of calcifications for breast biopsy”); U.S. Pat. No. 6,236,875 (“Surgical navigation systems including reference and localization frames”); U.S. Pat. No. 6,174,291 (“Optical biopsy system and methods for tissue diagnosis”).

Alternatively or additionally, some such media may include or otherwise interact with one or more modules 1625 for configuring or otherwise implementing optical and/or imaging protocols as described herein. See, e.g., U.S. Pat. No. 7,368,694 (“Device for measuring light absorption characteristics of a biological tissue sample”); U.S. Pat. No. 7,186,556 (“Modulating transcription of genes in vascular cells”); U.S. Pat. No. 6,816,564 (“Techniques for deriving tissue structure from multiple projection dual-energy x-ray absorptiometry”); U.S. Pat. No. 6,671,526 (“Probe and apparatus for determining concentration of light-absorbing materials in living tissue”); U.S. Pat. No. 6,366,635 (“Method and Apparatus for Three-Dimensional Image-Rendering of a Spatial and Tissue-Based Configuration Through Separating High Contrast and Injected Contrast Agents in Multi-Angular X-Ray Absorption Measurement”); U.S. Pat. No. 6,298,253 (“Method and device for measuring the absorption of radiation in a portion of tissue”); U.S. Pat. No. 6,198,949 (“Solid-state non-invasive infrared absorption spectrometer for the generation and capture of thermal gradient spectra from living tissue”); U.S. Pat. No. 6,050,947 (“Method and apparatus for harmonic tissue imaging and contrast imaging using coded transmission”); U.S. Pat. No. 5,719,399 (“Imaging and characterization of tissue based upon the preservation of polarized light transmitted therethrough”); U.S. Pat. No. 5,666,952 (“Tissue transmitted light sensor”); U.S. Pat. No. 7,230,242 (“Methods for SEM inspection of fluid containing samples”); U.S. Pat. No. 7,129,473 (“Optical image pickup apparatus for imaging living body tissue”); U.S. Pat. No. 7,006,861 (“Method and apparatus for detecting electromagnetic reflection from biological tissue”); U.S. Pat. No. 6,912,412 (“System and methods of fluorescence, reflectance and light scattering spectroscopy for measuring tissue characteristics”); U.S. Pat. No. 6,720,547 (“System and method for enhancing confocal reflectance images of tissue specimens”); U.S. Pat. No. 6,697,652 (“Fluorescence, reflectance and light scattering spectroscopy for measuring tissue”); U.S. Pat. No. 6,675,029 (“Apparatus and method for quantification of tissue hydration using diffuse reflectance spectroscopy”); U.S. Pat. No. 6,272,374 (“Method and apparatus for detecting electromagnetic reflection from biological tissue”); U.S. Pat. No. 6,110,117 (“Ultrasonic imaging method and image for doppler tissue parameters”).

Some variants may likewise include software-controlled or other special-purpose circuitry for categorically or otherwise indicating a shape of a cell group, a portion of an image, or some other item of interest on the order of a micron or longer. In light of teachings herein, numerous existing techniques may be applied for relating morphological categories or other such output from one or more modules 1624 of evaluation logic 1620 to such attributes as described herein without undue experimentation. See, e.g., U.S. Pat. No. 7,416,550”); U.S. Pat. No. Method and apparatus for the control and monitoring of shape change in tissue”); U.S. Pat. No. 7,343,190”); U.S. Pat. No. System and method for assessing fetal abnormality based on landmarks”); U.S. Pat. No. 7,316,904”); U.S. Pat. No. Automated pap screening using optical detection of HPV with or without multispectral imaging”); U.S. Pat. No. 7,252,638”); U.S. Pat. No. Method and system for simultaneously displaying relationships of measurements of features associated with a medical image”); U.S. Pat. No. 7,230,242”); U.S. Pat. No. Methods for SEM inspection of fluid containing samples”); U.S. Pat. No. 7,212,660”); U.S. Pat. No. System and method for finding regions of interest for microscopic digital montage imaging”); U.S. Pat. No. 7,102,740”); U.S. Pat. No. Method and system for determining surface feature characteristics using slit detectors”); U.S. Pat. No. 6,975,899”); U.S. Pat. No. Multi-modal optical tissue diagnostic system”); U.S. Pat. No. 6,288,539”); U.S. Pat. No. System for measuring an embryo, reproductive organs, and tissue in an animal”); U.S. Pat. No. 6,181,811”); U.S. Pat. No. Method and apparatus for optimizing biological and cytological specimen screening and diagnosis”); U.S. Pat. No. 6,084,407”); U.S. Pat. No. System for measuring tissue size and marbling in an animal”).

Alternatively or additionally, such media may bear one or more size-descriptive quantities characterizing an organelle of, a group of, a sample of, an image of, or some other aspect of one or more cells. In light of teachings herein, numerous existing techniques may be applied for relating such output from one or more modules 1626 of evaluation logic 1620 to such attributes as described herein without undue experimentation. See, e.g., U.S. Pat. No. 7,343,190 (“System and method for assessing fetal abnormality based on landmarks”); U.S. Pat. No. 7,252,638 (“Method and system for simultaneously displaying relationships of measurements of features associated with a medical image”); U.S. Pat. No. 6,833,242 (“Methods for detecting and sorting polynucleotides based on size”); U.S. Pat. No. 6,794,987 (“Object detection system and method of estimating object size”); U.S. Pat. No. 6,288,539 (“System for measuring an embryo, reproductive organs, and tissue in an animal”); U.S. Pat. No. 6,236,458 (“Particle size distribution measuring apparatus, including an array detector and method of manufacturing the array detector”); U.S. Pat. No. 6,137,407 (“Humanoid detector and method that senses infrared radiation and subject size”); U.S. Pat. No. 6,084,407 (“System for measuring tissue size and marbling in an animal”); U.S. Pat. No. 5,917,934 (“Automated visual inspection apparatus for detecting defects and for measuring defect size”).

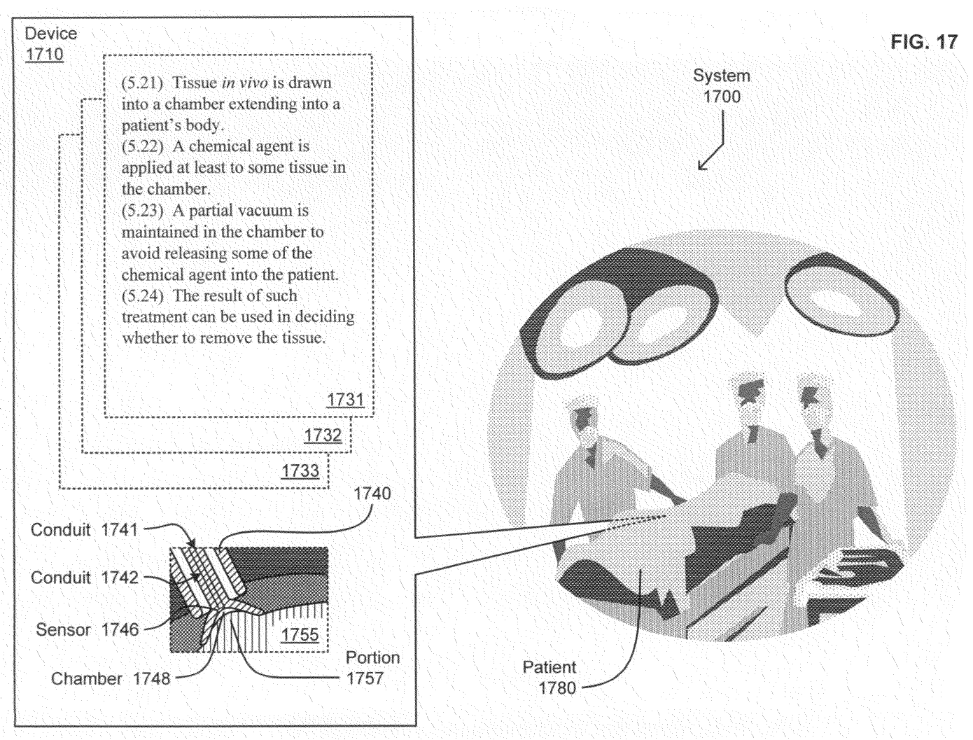

With reference now to FIG. 17, shown is a context in which one or more technologies may be implemented for performing one or more protocols 1731, 1732, 1733. In protocol 1731, for example, (5.21) tissue in vivo is drawn into a chamber extending into a patient's body; (5.22) a chemical agent is applied at least to some tissue in the chamber; (5.23) a partial vacuum is maintained in the chamber to avoid releasing some of the chemical agent into the patient; and (5.24) the result of such treatment can be used in deciding whether to remove the tissue. In system 1700, for example, a surgeon urges a distal portion 1740 of a laparoscopic or other device 1710 so that a flexible cup can extend into patient 1780 and into tissue 1755 as shown. A vacuum is drawn via one or more conduits 1741 so that a portion 1757 of tissue 1755 in vivo enters chamber 1748, bringing a surface of the tissue closer (to one or more sensors 1746 adjacent the chamber, e.g.). Some variants feature permeabilizing or other chemical agents in contact with the tissue portion 1757 in the chamber 1748 (through one or more of the conduits, for example, or otherwise positioned within the chamber). Alternatively or additionally, a succession of such agents may be brought into contact with the tissue portion, permitting a surgeon to image or otherwise observe the tissue in various sequential and/or conditional ways.

An embodiment provides one or more conduits 1742 or other physical media bearing one or more device-detectable measurements 1661, images 1662, intensity levels, or other forms of data indicating one or more chemical, therapeutic, and/or other treatments of an attached portion 1757, sample, or other component of tissue 1755 in a chamber 1748 extended into tissue 1755 of a patient 1780 or other subject. In some contexts, for example, at least one such medium bears a data component that was generated while the treatment was applied to such a tissue portion and/or extraction. Alternatively or additionally, in some variants, at least one such medium bears a Boolean computation or other result 1663 derived (by detection circuitry as described herein, e.g.) from raw data at sensor 1746.

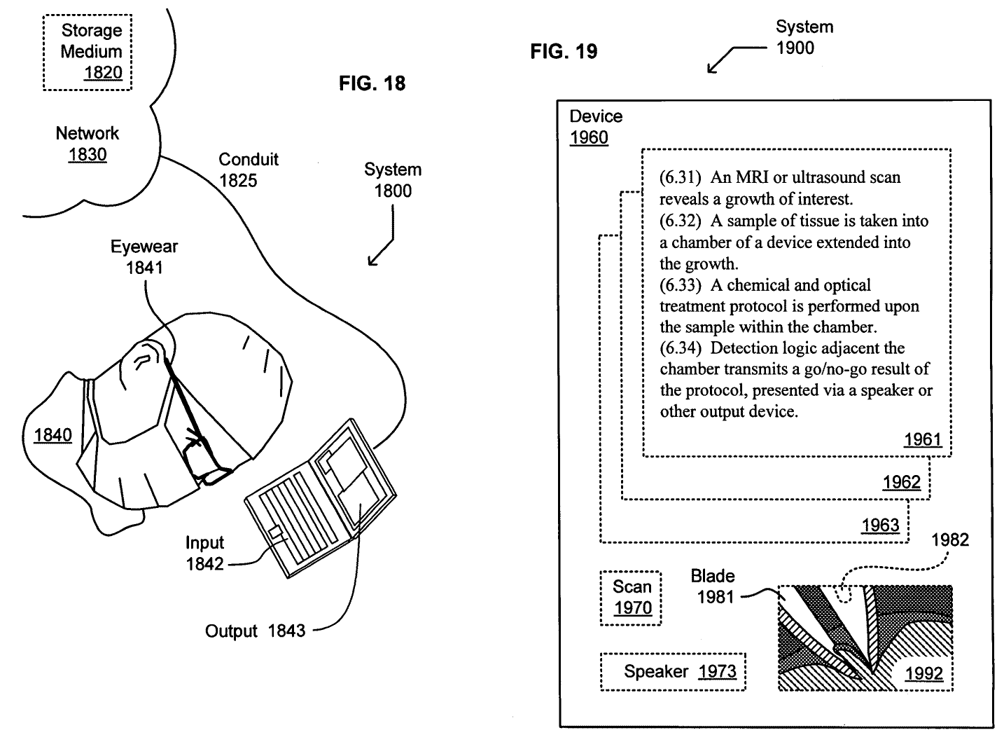

With reference now to FIG. 18, shown is a context in which one or more. technologies may be implemented for using any of the above-described protocols, devices, or other configurations. A surgeon 1840 may manipulate a sensor-containing probe or take other actions that provide input 1842 to system 1800 so that one or more conduits 1825, storage media 1820, other participants or other resources in network 1830 have access to transmitted results. In some contexts, such entities may respond by transmitting an apparent tissue category 122 (“malignant” or “unknown,” e.g.), an identifier 141 or other recommendation 143, one or more images or other results 192, 193 of an image processing or other computational protocol 115, or other such output 1843 of potential utility in a procedure being performed by surgeon 1840.

An embodiment provides one or more storage media 1820 or conduits 1825 bearing subject tissue data 1472 or other image data 1493 clearly depicting at least some of a cell to which one or more stains 644 or other agents 213, 893 effective for optical enhancement was applied in vivo. This can occur, for example, in a context in which such image data is transmitted to a surgeon via imaging eyewear 1841, projection surfaces 392, display outputs 1843, or other such presentation media. Alternatively or additionally, such embodiments may include one or more dispensers 891, 922, 1540 containing optical enhancement materials and one or more sensors 1242, 1331, 1644, 1746 or other circuitry for transmitting such device-detectable images.

With reference now to FIG. 19, shown is a context in which one or more technologies may be implemented for performing one or more protocols 1961, 1962, 1963. In protocol 1961, for example, (6.31) a magnetic resonance image or ultrasound scan reveals a growth of interest; (6.32) a sample of tissue is taken into a chamber of a device extended into the growth; (6.33) a chemical and optical treatment protocol is performed upon the sample within the chamber; and (6.34) detection logic adjacent the chamber transmits a go/no-go result of the protocol, presented via a speaker or other output device. In an instance of system 1900 in which scan 1970 reveals a growth of interest, for example, a surgical device 1960 can be extended into tissue 1992 to take a sample into a chamber (formed by one or more blades 1981 of the device 1960, e.g.) within which the chemical and/or optical treatments are performed. An ultrasound sensor or other sensor 1982 (adjacent the chamber, e.g.) may, in some variants, work in conjunction with a software or other remote module 1158 of pattern recognition logic 1150 or other detection logic configured to transmit a go/no-go result. In a context in which surgeon 1840 selects a given decision protocol 1962, this result can signify whether the selected protocol's suggestion of whether to extract the tissue 1992. An affirmative indicator 161 can, for example, be transmitted as a spoken “yes” or beep via an earpiece or other speaker 1973, or a blue indicator light in the surgeon's field of view. In a context in which a protocol within the chamber takes about a second or more, an contingent negative indicator can likewise be transmitted (as a spoken “no” or red light, e.g.). Such suggestions can, in many contexts, facilitate a faster execution of a surgical procedure in which two or more regions of tissue are to be investigated.

Alternatively or additionally, some embodiments, may provide a dispenser 921, 922, 1540 configured to apply a treatment material to tissue 985, 1531 of an organism 1210 in vivo; a protrusion 1520 or other cooling component configured to freeze at least some of the tissue 985, 1531 in vivo; and a blade 1981, rotary cutting element, retractable element, or other extraction element configured to remove at least a portion of the tissue from the organism.

In some variants, one or more results 192, 872 can comprise go/no-go indications of (a) whether tissue apparently exhibits a pathology, (b) whether tissue apparently exhibits a chromosomal attribute of interest, (c) whether a fraction of tissue apparently meets a profile exceeds a threshold, (d) whether other thresholds 1171 or criteria 1172 are met, (e) whether an extraction meets a standard profile 1184, (f) whether a selected profile 1185 specified by a pathologist 471 or other expert are met, and/or (g) other such logical expressions. Such results may be indicated by a color, symbol, or other expression in real time via a surgeon's eyewear 1841 or other device 1960 in some contexts, for example, or via some other such mode of output.

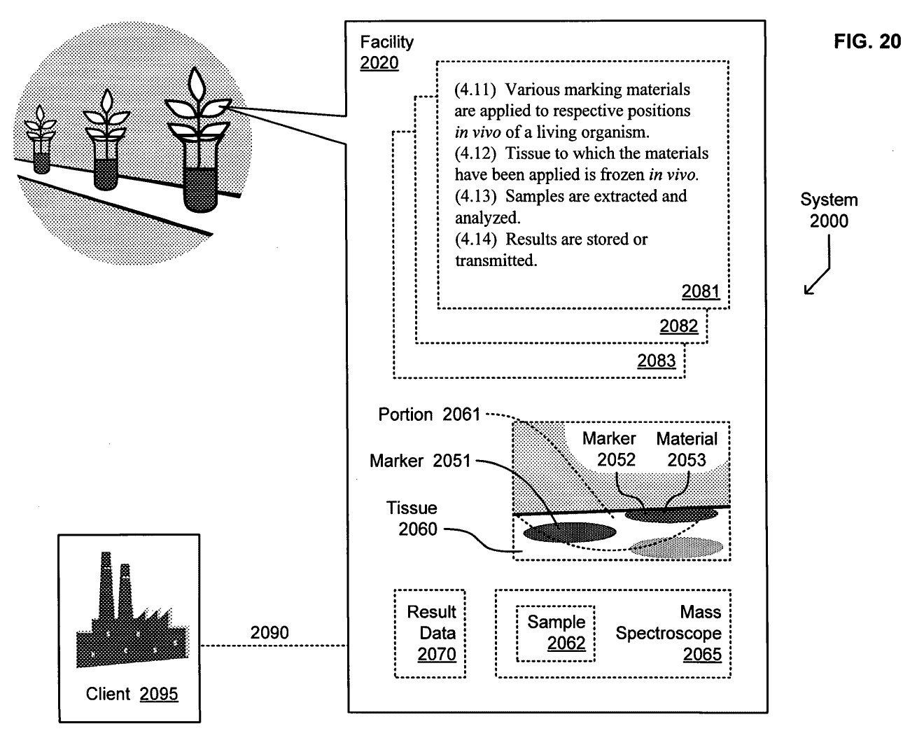

With reference now to FIG. 20, shown is a facility 2020 or other context in which one or more technologies may be implemented for performing one or more evaluation protocols 2081, 2082, 2083. In protocol 2081, for example, (4.11) various marking materials are applied to respective positions in vivo of a living subject; (4.12) tissue to which the materials have been applied is frozen in vivo; (4.13) samples are extracted and analyzed; (4.14) results are stored or transmitted. In an instance of system 2000 in an agricultural research facility, for example, various formulations of markers 2051, 2052 or other materials 2053 may be applied to respective positions of plant tissue 2060 in vivo. Effects of such materials may be evaluated, for example, by freezing and extracting one or more portions 2061 of such tissue in vivo, potentially without any substantial harm to the organism organism. Samples 2062 of such tissue 2062 may then be analyzed (in a microscope or mass spectroscope 2065, e.g.), and result data 2070 sent (via conduit 2090, e.g.) to clients 2095 or other recipients.

In some embodiments, an “extraction” of frozen tissue may include fine slices of the tissue (obtained by a laser microtome or ultramicrotome, e.g.), whole cells, cytoplasmic or other fluid samples, protein or other molecular fragments (observable by electrospray mass spectrometry, e.g.), or other such forms of matter.

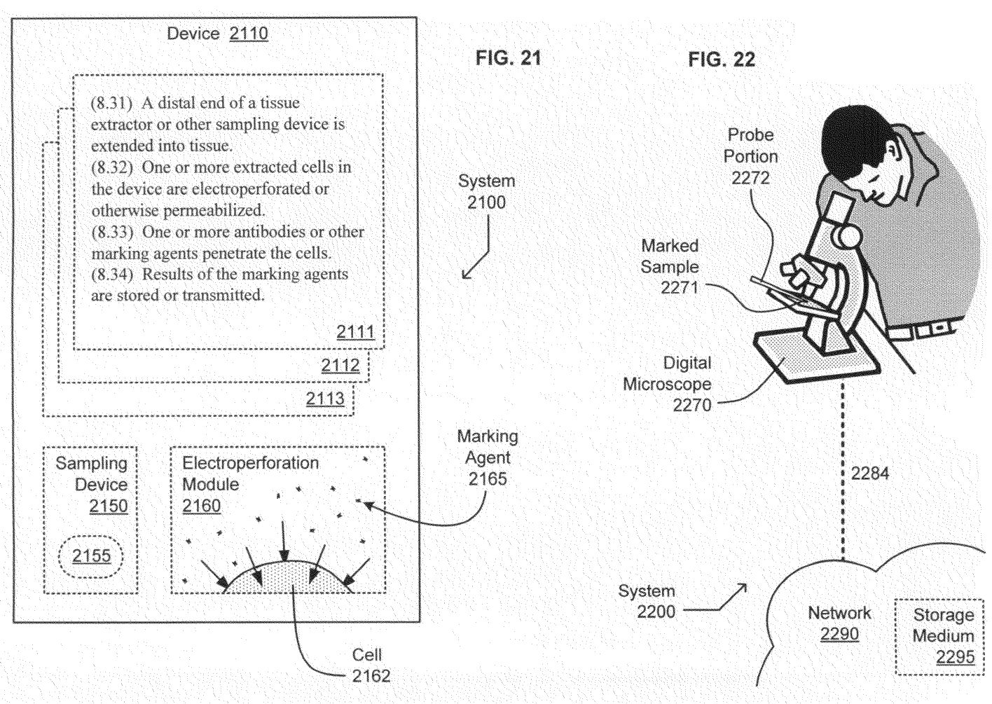

With reference now to FIG. 21, shown is a context in which one or more technologies may be implemented for performing one or more protocols 2111, 2112, 2113. In protocol 2111, for example, (8.31) a distal end of a tissue extractor or other sampling device is extended into tissue; (8.32) one or more extracted cells in the device are electroperforated or otherwise permeabilized; (8.33) one or more antibodies or other marking agents penetrate the cells; and (8.34) results of the marking agents are stored or transmitted. In some devices 2110 of system 2100, for example, a permeabilizing agent, electroperforation module 2160, or other such component effectively permits one or more marking agents 2165 or other materials to enter one or more cells 2162 (in a chamber 2155 of a sampling device 2150, e.g.).

In light of teachings herein, numerous existing techniques may be applied for temporarily or otherwise permeabilizing an organic membrane to facilitate marking or other operations as described herein without undue experimentation. See, e.g., U.S. Pat. No. 7,412,284 (“Medical or veterinary system for electroporation mediated delivery for drugs and genes”); U.S. Pat. No. 7,393,680 (“Combined electroporation and microinjection method for the penetration of lipid bilayer membranes”); U.S. Pat. No. 7,306,940 (“Electroporation device and method, delivering a modulated signal under continuous control of cell electropermeabilization”); U.S. Pat. No. 7,271,005 (“Modulation of bacterial membrane permeability”); U.S. Pat. No. 7,186,559 (“Medical or veterinary system and method for electroporation of biological samples”); U.S. Pat. No. 6,846,668 (“Microfabricated cell injector”); U.S. Pat. No. 6,706,088 (“Method for controlling membrane permeability by microwave and method for producing organic separation membrane”); U.S. Pat. No. 6,589,503 (“Membrane-permeant peptide complexes for medical imaging, diagnostics, and pharmaceutical therapy”); U.S. Pat. No. 6,319,901 (“Methods for prolonging cell membrane permeability”); U.S. Pat. No. 6,015,834 (“In vivo treatment of mammalian cells with a cell membrane permeant calcium buffer”).

With reference now to FIG. 22, shown is a context in which one or more technologies may be implemented. An embodiment provides a conduit 2284 or storage medium 2295 bearing cell attribute indicators 1050 or other device-detectable data from a digital microscope 2270 or other such equipment. Such equipment may be configured (a) to observe a marked sample 2271 in a probe portion 2272 implementing an extraction module 540, 660, 850 suitable for extending into an organism's tissue and (b) to transmit a result 872, 1194, 1687 of one or more therapeutic agents 333, marking agents 2165, or other agents 893 having been applied (adjacent device 2110, e.g.) to a portion of tissue in or from the organism.

In some variants, a surgical instrument or other device 2110 includes one or more primary chamber 2155 and an electroperforation module 2160 or other treatment modules 530, 890 configured to apply electrical, optical, or other treatments to a tissue sample or other extraction in the chamber(s). Such configurations may likewise include a camera or other output module configured to transmit a result of such treatments (to network 2290, e.g.).

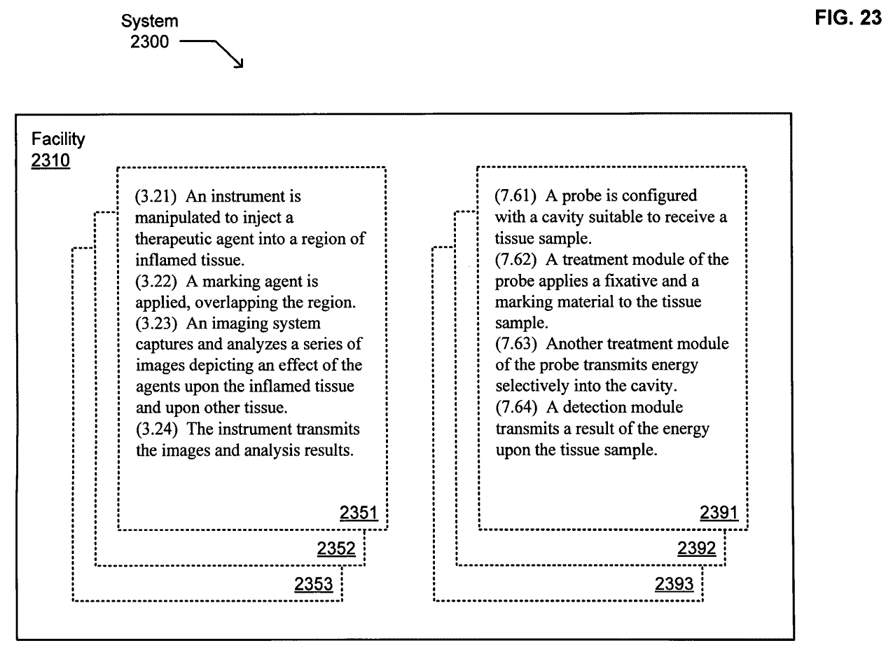

With reference now to FIG. 23, shown is a context in which one or more technologies may be implemented for performing one or more protocols 2351, 2352, 2353, 2391, 2392, 2393. In protocol 2351, for example, (3.21) an instrument is manipulated to inject a therapeutic agent into a region of inflamed tissue; (3.22) a marking agent is applied, overlapping the region; (3.23) an imaging system captures and analyzes a series of images depicting an effect of the agents upon the inflamed tissue and upon other tissue; and (3.24) the instrument transmits the images and analysis results. In a context in which one or more systems 1100, 1400 are implemented in facility 2310, for example, an analyst may invoke one or more modules 1159 of image enhancement software or other pattern recognition logic 1150 for analyzing a series of images 1191 depicting an artificially or otherwise visible effect of the agents upon inflamed cells and/or upon other tissue across a period of several minutes or hours, for example.

Alternatively or additionally, for example, facility 2310 may implement protocol 2391, in which (7.61) a probe is configured with a cavity suitable to receive a tissue sample; (7.62) a treatment module of the probe applies a fixative and a marking material to the tissue sample; (7.63) another treatment module of the probe transmits energy selectively into the cavity; and (7.64) a detection module transmits a result of the energy upon the tissue sample. In a context in which one or more systems 300, 600 are implemented in facility 2310, for example, an applicator 340 or other component may cause one or more fixatives 331, 641; stains 644 or other marking agents 332; or other materials to come into contact with tissue before, during, after, or interleaved with an extraction of such tissue into a chamber 635. A light 345 or other treatment module may then transmit energy selectively into the cavity, such as to minimize an exposure of subject 380 or other individuals to such energy.

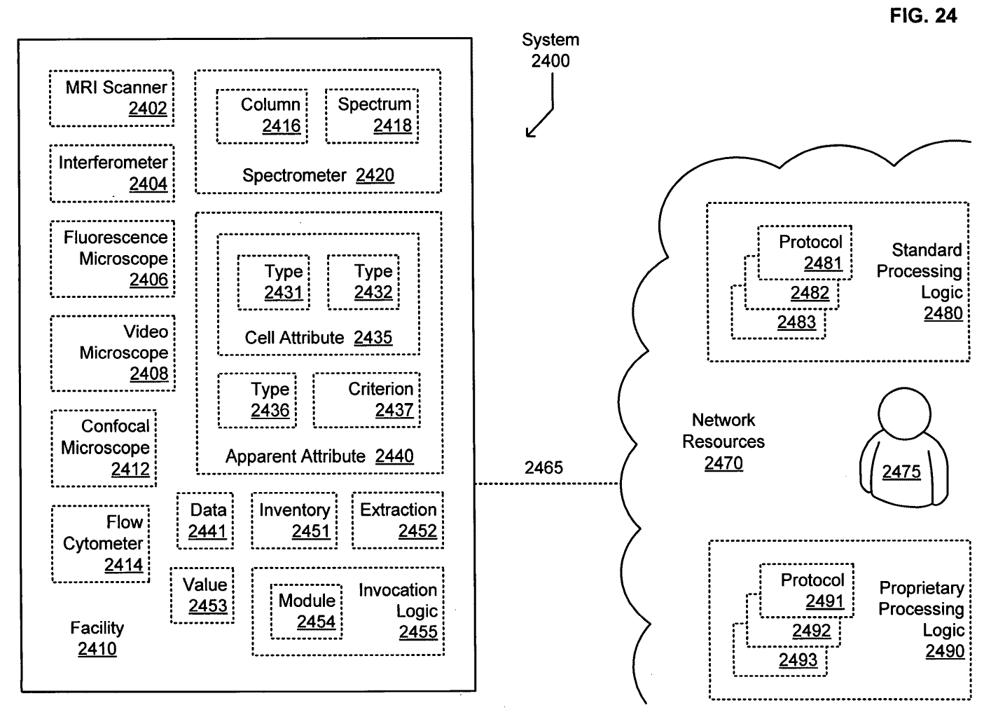

With reference now to FIG. 24, shown is a context in which one or more technologies may be implemented. A facility 2410 may include or otherwise interact with one or more MRI scanners 2402, interferometers 2404, fluorescence microscopes 2406, video microscopes 2408, flow cytometers 2412, confocal microscopes 2414, spectrometers 2420, extraction modules, or other such instruments. In some variants, for example, such equipment may be configured to implement preliminary protocols, to generate raw sensor data, or otherwise to facilitate clinicians or other local users determining apparent attributes 2440 of various tissues 240, 520, 640, 985, 1531, 2060; treatments; or extractions 2452 as described herein.

In some protocols, for example, a user and/or device in facility 2410 applies one or more criteria 2437 locally for making a preliminary determination of an “apparently irregular” or other chromosome type 2431 or other cell attribute 2435. One or more modules 2454 of invocation logic 2455 may respond, for example, by selecting one or more providers 2475 or other network resources 2470 or otherwise by triggering an evaluation process.

An embodiment provides one or more conduits 2465 bearing one or more of raw sensor data, records 111, image data 871, organelle morphologies or other types 2432 indicating cell attributes 2435, identifiers of protocols used, or other types 2436 or apparent attributes 2440 of data resulting from or otherwise indicating (a) an optical enhancement or other chemical treatment material applied in vivo, (b) a freezing agent or other fixative applied in vivo, and/or (c) an extraction of treated tissue from an organism. In some variants, such signals may directly invoke one or more protocols 2481, 2482, 2483 of spectral karyotyping pseudo-coloring, BLAST searching or other sequence analysis, or other common or standard processing logic 2480. Alternatively or additionally, such signals may likewise permit a server or provider 2475 to implement one or more protocols 2491, 2492, 2493 of custom image processing, advanced diagnostic services, or other such specialized or proprietary processing logic 2490. In any case, such network resources may respond with updated criteria or protocols 114 for use by facility 2410, with a specification of or response to a material or equipment inventory 2451, or with other images 124, measurements 153, diagnoses, recommendations 144, authorizations, or other such feedback for use in facility 2410.

Alternatively or additionally, some variants may include a probe comprising a first dispenser 921, 922, 1540 configured to apply a first treatment material to tissue 985, 1531 of an organism 1210 in vivo, a first optical element configured to transmit light into the tissue 985, 1531 of the organism in vivo, and a display or other output module configured to transmit a result 1194, 1663 of at least the light and the first treatment material upon the tissue of the organism in vivo. Alternatively or additionally, such embodiments may include one or more physical media 100, 1000 bearing an image 1662 (from a confocal microscope 2412 or other laser-scanning optical module 1650, e.g.) of at least some of a cell to which a material 213 was applied in vivo primarily for optical enhancement.

In light of teachings herein, numerous existing techniques may be applied for detecting luminescence in an imaging or other analytical protocol as described herein without undue experimentation. See, e.g., U.S. Pat. No. 7,372,985 (“Systems and methods for volumetric tissue scanning microscopy”); U.S. Pat. No. 7,336,989 (“System and method for quantitative or qualitative measurement of exogenous substances in tissue and other materials using laser-induced fluorescence spectroscopy”); U.S. Pat. No. 7,310,547 (“Fluorescent fiberoptic probe for tissue health discrimination”); U.S. Pat. No. 7,236,815 (“Method for probabilistically classifying tissue in vitro and in vivo using fluorescence spectroscopy”); U.S. Pat. No. 7,176,345 (“Transgenic animals expressing light-emitting fusion proteins and diagnostic and therapeutic methods therefor”); U.S. Pat. No. 7,139,598 (“Determination of a measure of a glycation end-product or disease state using tissue fluorescence”); U.S. Pat. No. 7,050,208 (“Scanning microscopy, fluorescence detection, and laser beam positioning”); U.S. Pat. No. 6,984,828 (“Quantified fluorescence microscopy”); U.S. Pat. No. 6,697,652 (“Fluorescence, reflectance and light scattering spectroscopy for measuring tissue”); U.S. Pat. No. 6,631,289 (“System and method of fluorescence spectroscopic imaging for characterization and monitoring of tissue damage”); U.S. Pat. No. 6,510,338 (“Method of and devices for fluorescence diagnosis of tissue, particularly by endoscopy”); U.S. Pub. No. 20070077639 (“Estimation of activity or inhibition of processes involved in nucleic acid modification using chemiluminescence quenching”). Alternatively or additionally, such techniques may be used for manipulating and/or observing such extractions 2452 or other forms of matter using a magnetic resonance imaging (MRI) scanner 2402, interferometer 2404, fluorescence microscope 2408, video microscope 2408, electron microscope 770, flow cytometer 2412, confocal microscope 2414, spectrometer 2420, or other such equipment as described herein. Images or other results from such equipment may be stored, presented, or otherwise transmitted on various conduits 2465 or other media 100, 1000 as described herein, for example. Alternatively or additionally, in some variants, probes or other components of such instruments may include one or more surfaces 214, 574, 584, 1630 configured to permit a surgeon to extend extraction modules or other probes into a subject organism.

With reference now to FIG. 25, shown is a flow 2500 comprising operation 2560—obtaining device-detectable data indicating an extraction of chemically treated tissue frozen in vivo (e.g. provider 2475 or other network resources 2470 receiving image data 871, 1493 or other attribute indicators 1080 depicting a sample of tissue 240, 1532 that has been marked and then frozen in vivo before extraction). This can occur, for example, in a context in which facility 2410 transmits subject tissue data 1472 or other result data 1494 from within a probe 1510 or other such on-site equipment. In some variants, for example, facility 2410 may comprise a hospital at which a human or other subject 280 undergoes surgery. Alternatively or additionally, operation 2560 may include one or more instances of operation 2520—generating at least some of the device-detectable data (e.g. provider 2475 deriving one or more images 1011 or other cell attribute indicators 1050 with one or more protocols 2482, 2492 of processing logic). This can occur, for example, in a context in which such extractions comprise a bodily fluid or other sub-cellular material containing molecular components of interest and in which facility 2410 performs some data acquisition on behalf of provider 2475. Alternatively or additionally, provider 2470 may furnish facility 2410 with an inventory 2451 of suitable reagents for use in proprietary protocols, with or without revealing their composition. Flow 2500 further includes operation 2580—transmitting an evaluation of the device-detectable data (e.g. provider 2475 transmitting a summary, a responsive record 111, a diagnosis, a category 121, an estimate, or other such indicators 162 as described herein).

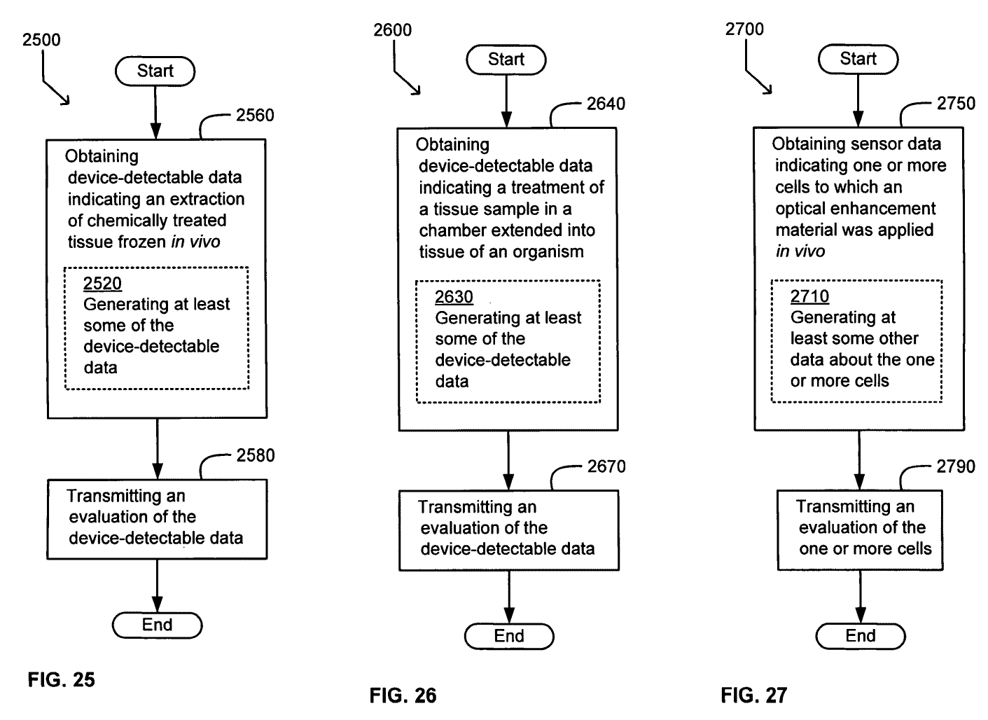

With reference now to FIG. 26, shown is a flow 2600 comprising operation 2640-obtaining device-detectable data indicating a treatment of a tissue sample in a chamber extended into tissue of an organism (e.g. evaluation module 1140 or other such resources receiving measurements 153, images 1191, pathological data, or other such information that includes data from sensors 553, 1644, 1746 about samples 552, 1112, 2062 of tissue 240, 1755, 2060). This can occur, for example, in a context in which a surgical probe or other device 610, 800, 1330, 1710 configured for tissue extraction has such sensors positioned adjacent an extraction module 540, 660, 850 or other such recessed portion. Alternatively or additionally, operation 2640 may include one or more instances of operation 2630—generating at least some of the device-detectable data (e.g. instrument 1110 monitoring sample 1112 during or after an optical, chemical, or other treatment). This can occur, for example, in a context in which a syringe or other instrument 1110 includes or otherwise interacts with a flow cytometer 2412, spectrometer 2420, or imaging system as described herein. Alternatively or additionally, instrument 1110 may include or otherwise interact with invocation logic 2455 configured to request or otherwise trigger evaluation by one or more modules 1157 of pattern recognition logic 1150, provider 2475, or other such resources. Flow 2600 further includes operation 2670—transmitting an evaluation of the device-detectable data (e.g. evaluation module 1140 transmitting one or more images 1191, types 1192, values 1193, diagnoses, or other results 1194 from the device-detectable data conforming to one or more pathological profiles 1182). This can occur, for example, in a context in which evaluation module 1140 responds to such invocations within a few minutes, for example, optionally by applying one or more protocols 2493 of proprietary processing logic 2490 in a highly specialized and central facility.

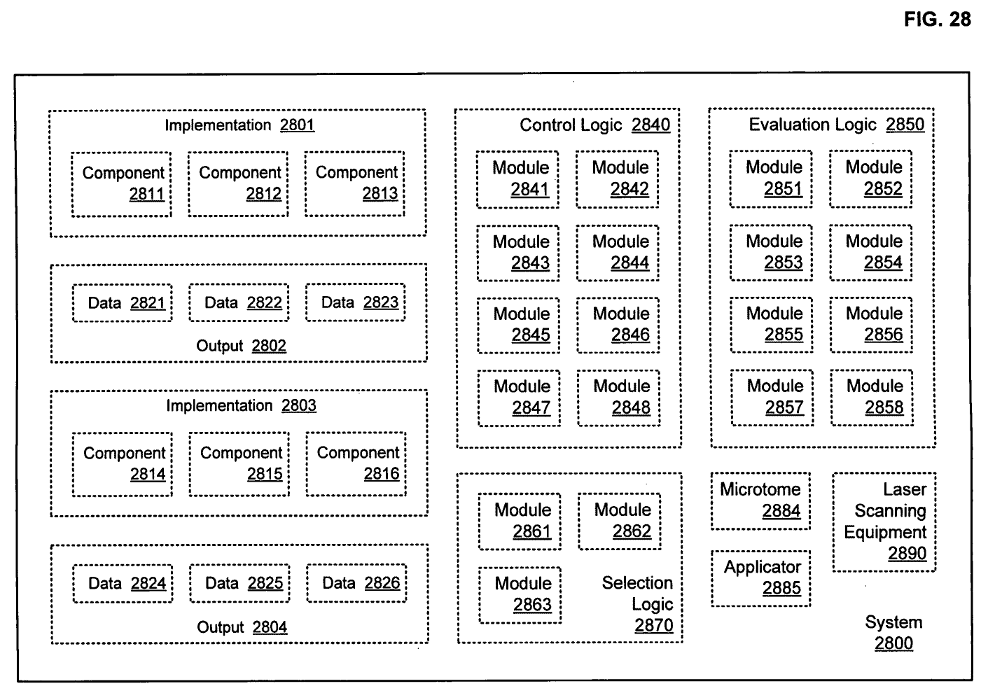

With reference now to FIG. 27, shown is a flow 2700 comprising operation 2750—obtaining a device-detectable image of at least some of a cell to which an optical enhancement material was applied in vivo (e.g. pattern recognition logic 1150 or other evaluation logic 1620 receiving such images 1191 from a confocal microscope 2414, a charge-coupled device, or other such optical modules 1640). This can occur, for example, in a context in which a vital stain or other marking agent 2165 has been accepted into the cell(s) 2162 in vivo, in which such equipment is configured to observe the cell(s) in vivo or in a chamber of probe 1610, and in which such evaluation modules 1140 or other resources include an image recognition module 1152 or other protocols for image analysis. Alternatively or additionally, operation 2750 may include one or more instances of operation 2710—generating at least some other device-detectable data (e.g. one or more instruments 1110, sensors 1746, inputs 1842, or other components providing one or more concentrations 151 or other measurements 153, phenotypes, physiological responses 355, symptoms, protocols 1122, or other such supplemental input 110 from a clinician 1490, subject, or other user). This can occur, for example, in a context in which one or more modules 1157 of pattern recognition logic 1150 queries such a user in response to image recognition module 1152 reporting a success or failure in locating a key feature in the image(s) of the cell(s), for example. In a context in which module 1156 recognizes a dark-field image, for example, pattern recognition logic 1150 may respond by triggering a user query as to (a) whether an image is from a fluorescence microscope 2406 or other recognized equipment, (b) which protocols were applied, (c) who or what performed the protocols, (d) where and when such protocols were applied, (e) what pathological indicators were present, or other such result-determinant data. Alternatively or additionally, one or more records 1690 may include data indicative of one or more medicants administered by port 1674, one or more treatments administered by an emitter 1642 or thermal element 1672, or other such data potentially affecting one or more modules 1621-1626 of evaluation logic 1620 or other processing logic. Flow 2700 further includes operation 2790—transmitting an evaluation of the device-detectable image (e.g. one or more protocols 2481, 2491 of standard processing logic 2481, proprietary processing logic 2490, or other network resources 2470 transmitting one or more categories 121, estimates, sizes 1031, morphologies 1032, chromosomal patterns 1040, or other such cell attribute indicators at least partly derived from cell images). This can occur, for example, in a context in which such image data 1493 depicts one or more cell features with sufficient clarity to facilitate a diagnosis or other inference and in which system 2400 includes or otherwise interacts with one or more components of system 1100 via one or more conduits 2465 or other media 100, 1000.

With reference now to FIG. 28, shown is a distributed or other system 2800 comprising one or more implementations 2801, 2803; one or more modules 2841, 2842, 2843, 2844, 2845, 2846, 2847, 2848 of control logic 2840; one or more modules 2851, 2852, 2853, 2854, 2855, 2856, 2857, 2858 of evaluation logic 2850; or one or more modules 2861, 2862, 2863 of selection logic 2870. Such implementations may include one or more microtomes 2884 or other material handling equipment, one or more applicators 2885 or other tissue or extraction treatment equipment; laser scanning equipment 2890 or other imaging or measurement equipment, or other such components 2811, 2812, 2813, 2814, 2815, 2816 for interacting with such logic and/or generating data 2821, 2822, 2823, 2824, 2825, 2826 as described below.

Referring again to FIG. 27, some instances of flow 2700 may be implemented entirely within system 2800. Operation 2750 may be implemented by configuring one or more sensors or other components 2814, 2815 as logic for obtaining device-detectable images or other data indicating tissue to which an optical enhancement material was applied in vivo, for example, such as by including special-purpose instruction sequences or special-purpose-circuit designs for this function. Output data 2824, 2825 from such a component in system 2800 may (optionally) be recorded or presented locally. Component 2815 may perform operation 2710 via implementation as logic for generating at least some of the device-detectable data, for example. Implementation output data 2825 from such a component in system 2800 may likewise be recorded locally or transmitted one or more media 100,1000, for example. Component 2816 may perform operation 2790 via implementation as logic for transmitting an evaluation of the device-detectable data. Output 2804 from flow 2700 may likewise include other data 2826 as described herein. Each portion of implementation 2803 may likewise include one or more instances of software, hardware, or the like implementing logic that may be expressed in several respective forms as described herein or otherwise understood by those skilled in the art.

Referring again to FIG. 27, some instances of flow 2700 may be implemented entirely within system 2800. Operation 2750 may be implemented by configuring one or more sensors or other components 2814, 2815 as logic for obtaining device-detectable data including (a) an earlier image depicting at least some of a cell to which an optical enhancement material was applied in vivo and (b) a later image depicting at least some of the cell to which the optical enhancement material was applied in vivo, for example. Output data 2824, 2825 from such a component in system 2800 may be recorded or displayed locally one such media, in some variants. Component 2815 may perform operation 2710 via implementation as logic for generating at least some of the device-detectable data, for example. Implementation output data 2825 from such a component in system 2800 may likewise be recorded locally or transmitted one or more media 100, 1000, for example. Component 2816 may perform operation 2790 via implementation as logic for transmitting an evaluation of the device-detectable data. Output 2804 from flow 2700 may likewise include other data 2826 as described herein. Each portion of implementation 2803 may likewise include one or more instances of software, hardware, or the like implementing logic that may be expressed in several respective forms as described herein or otherwise understood by those skilled in the art.

Firstly, referring again to FIGS. 5 & 12, some embodiments may include software-controlled or other special-purpose circuitry for positioning or otherwise configuring a surgical or other instrument with removable components. In light of teachings herein, numerous existing techniques may be applied for implementing such modules 2841 of software or other control logic 2840 as described herein without undue experimentation. See, e.g., U.S. Pat. No. 7,367,973 (“Electro-surgical instrument with replaceable end-effectors and inhibited surface conduction”); U.S. Pat. No. 7,179,263 (“Methods and instruments for laparoscopic spinal surgery”); U.S. Pat. No. 7,008,431 (“Configured and sized cannula”); U.S. Pat. No. 6,974,483 (“Modular neck for femur replacement surgery”); U.S. Pat. No. 6,692,514 (“Surgical clamp having replaceable pad”); U.S. Pat. No. 6,595,984 (“Laparoscopic instrument with a detachable tip”); U.S. Pat. No. 6,464,704 (“Bipolar electrosurgical instrument with replaceable electrodes”); U.S. Pat. No. 6,293,954 (“Surgical clamp with replaceable clamp members”); U.S. Pat. No. 6,197,002 (“Laparoscopic tool and method”); U.S. Pat. No. 6,174,291 (“Optical biopsy system and methods for tissue diagnosis”); U.S. Pat. No. 5,893,875 (“Surgical instrument with replaceable jaw assembly”). Alternatively or additionally, such modules may comprise or otherwise interact with circuitry for positioning a distal portion 550, dispenser, or entirety of a probe 590 or other instrument 1260.

Secondly, some variants may include handling and/or imaging devices configured to facilitate observation of tissue 240, 1531, 1755, 2060 or other forms of matter, with or without fixatives 331, 641 or other such delay-inducing treatments. In light of teachings herein, numerous existing techniques may be applied for implementing one or more modules of 2843 of control logic 2840 for such functions as described herein without undue experimentation. See, e.g., U.S. Pat. No. 7,347,817 (“Polarized in vivo imaging device, system and method”); U.S. Pat. No. 7,303,741 (“Systems and methods for high-resolution in vivo imaging of biochemical activity in a living organism”); U.S. Pat. No. 7,267,648 (“Magnifying image pickup unit for an endoscope, an endoscope for in vivo cellular observation that uses it, and endoscopic, in vivo cellular observation methods”); U.S. Pat. No. 7,230,242 (“Methods for SEM inspection of fluid containing samples”); U.S. Pat. No. 7,009,634 (“Device for in-vivo imaging”); U.S. Pat. No. 6,611,716 (“Multi-phasic microphotodiode retinal implant and adaptive imaging retinal stimulation system”); U.S. Pat. No. 6,546,272 (“Apparatus for in vivo imaging of the respiratory tract and other internal organs”); U.S. Pat. No. 6,296,608 (“Diagnosing and performing interventional procedures on tissue in vivo”); U.S. Pat. No. 7,411,672 (“Method and apparatus for chemical imaging in a microfluidic circuit”); U.S. Pat. No. 7,391,936 (“Microfluidic sensors and methods for making the same”); U.S. Pat. No. 7,214,298 (“Microfabricated cell sorter”); U.S. Pat. No. 7,160,730 (“Method and apparatus for cell sorting”); U.S. Pat. No. 6,897,031 (“Multiparameter FACS assays to detect alterations in exocytosis”); U.S. Pat. No. 6,692,952 (“Cell analysis and sorting apparatus for manipulation of cells”); U.S. Pat. No. 6,455,263 (“Small molecule library screening using FACS”); U.S. Pat. No. 5,985,216 (“Flow cytometry nozzle for high efficiency cell sorting”); U.S. Pat. No. 5,264,341 (“Selective cloning for high monoclonal antibody secreting hybridomas”). Alternatively or additionally, such modules may comprise or otherwise interact with optical modules 1650, microscopes, or other imaging equipment operable for receiving one or more separable extraction modules 660, 850 (of a probe, e.g.) that contain a chamber 955 or other such feature (configured to bear tissue 985, 2060, e.g.).

Thirdly, some variants may include software-controlled or other special-purpose circuitry for controlling one or more instances of microtomes 2884, applicators 2885 containing protein-dissolving materials, or other modes of extracting or dividing tissue samples. In light of teachings herein, numerous existing protocols may be applied for implementing one or more modules 2844 of control logic 2840 operable for such manipulation as described herein without undue experimentation. See, e.g, U.S. Pat. No. 7,354,775 (“Reagent for partially lysing a cell membrane of a red blood cell, a reagent for detecting malaria infected red blood cells, and a sample analyzing method for detecting malaria infected red blood cells”); U.S. Pat. No. 7,156,814 (“Apparatus and method for harvesting and handling tissue samples for biopsy analysis”); U.S. Pat. No. 7,115,386 (“Device and method for carrying out immunological marking techniques for thin-sectioned tissue”); U.S. Pat. No. 6,942,169 (“Micromachined lysing device and method for performing cell lysis”); U.S. Pat. No. 6,623,945 (“System and method for microwave cell lysing of small samples”); U.S. Pat. No. 6,558,629 (“Device and method for preparing tissue specimen for histologic sectioning”); U.S. Pat. No. 6,113,584 (“Intraluminal delivery of tissue lysing medium”); U.S. Pat. No. 6,035,258 (“Method for correction of quantitative DNA measurements in a tissue section”); U.S. Pat. No. 6,017,476 (“Method for embedding and sectioning specimen”).

Fourthly, some variants may include software-controlled or other special-purpose circuitry for causing a visible modification of a selected portion of tissue in vivo or otherwise. In light of teachings herein, numerous existing techniques may be applied for implementing such modules 2847, 2861 of selection logic 2870 or other control logic 2840 as described herein without undue experimentation. See, e.g., U.S. Pat. No. 7,332,360 (“Early detection of metal wiring reliability using a noise spectrum”); U.S. Pat. No. 7,329,414 (“Biodegradable polymer for marking tissue and sealing tracts”); U.S. Pat. No. 7,285,364 (“Permanent, removable tissue markings”); U.S. Pat. No. 7,127,040 (“Device and method for margin marking tissue to be radiographed”); U.S. Pat. No. 7,047,063 (“Tissue site markers for in vivo imaging”); U.S. Pat. No. 6,780,179 (“Methods and systems for in situ tissue marking and orientation stabilization”); U.S. Pat. No. 6,745,067 (“System for marking the locations of imaged tissue with respect to the surface of the tissue”); U.S. Pat. No. 6,464,646 (“Instrument and method for locating and marking a hot spot in a person's body tissue”); U.S. Pat. No. 6,432,064 (“Biopsy instrument with tissue marking element”); U.S. Pat. No. 6,394,965 (“Tissue marking using biocompatible microparticles”); U.S. Pat. No. 6,296,608 (“Diagnosing and performing interventional procedures on tissue in vivo”); U.S. Pat. No. 6,228,055 (“Devices for marking and defining particular locations in body tissue”); U.S. Pat. No. 5,690,107 (“Method for positioning and marking a patient at a diagnostic apparatus”). Alternatively or additionally, such modules may implement circuitry for causing a marking agent or other material to be applied to one or more cells in situ in response to user input. In some variants, for example, such an application may start or end within a few seconds or minutes of a user's “dispense now” signal.

Fifthly, some variants may include software-controlled or other special-purpose configurations for permitting optical or other equipment to receive an extraction module or other vessel as described herein. In light of teachings herein, numerous existing techniques may be applied for implementing such modules 2848 of control logic 2840 without undue experimentation. See, e.g., U.S. Pat. No. 7,411,672 (“Method and apparatus for chemical imaging in a microfluidic circuit”); U.S. Pat. No. 7,410,055 (“Transport container for slides for immunological labeling for thin tissue sections”); U.S. Pat. No. 7,364,655 (“Method and apparatus for injecting a sample into a chromatography system”); U.S. Pat. No. 7,361,305 (“Analyzer system having sample rack transfer line”); U.S. Pat. No. 7,273,759 (“Plate alignment and sample transfer indicia for a multiwell multiplate stack and method for processing biological/chemical samples using the same”); U.S. Pat. No. 7,230,242 (“Methods for SEM inspection of fluid containing samples”); U.S. Pat. No. 7,195,698 (“Capillary electrophoretic apparatus, sample plate and sample injection method”); U.S. Pat. No. 7,172,558 (“Device for containing and analyzing surgically excised tissue and related methods”); U.S. Pat. No. 6,939,452 (“Parallel sample loading and injection device for multichannel microfluidic devices”); U.S. Pat. No. 6,833,267 (“Tissue collection devices containing biosensors”); U.S. Pat. No. 6,384,418 (“Sample transfer apparatus and sample stage”); U.S. Pat. No. 6,372,182 (“Integrated body fluid collection and analysis device with sample transfer component”); U.S. Pat. No. 6,068,978 (“Apparatus and method for transfer of a fluid sample”).

Sixthly, some variants may indicate one or more genetic anomalies or other chromosomal patterns 1040 characterizing an image or other optical field of a sensor, an extraction, or another mode or region. In light of teachings herein, numerous existing techniques may be applied for relating such output from one or more modules 2851 of evaluation logic 2850 to such attributes as described herein without undue experimentation. See, e.g., U.S. Pat. No. 7,368,245 (“Method and probes for the detection of chromosome aberrations”); U.S. Pat. No. 7,303,880 (“Microdissection-based methods for determining genomic features of single chromosomes”); U.S. Pat. No. 7,205,109 (“Method for detecting hepatocarcinoma susceptibility by detecting a tumor related gene in the region of human chromosome 17 p. 13.3”); U.S. Pat. No. 7,176,345 (“Transgenic animals expressing light-emitting fusion proteins and diagnostic and therapeutic methods therefor”); U.S. Pat. No. 7,115,709 (“Methods of staining target chromosomal DNA employing high complexity nucleic acid probes”); U.S. Pat. No. 7,094,534 (“Detection of chromosoal abnormalities associated with breast cancer”); U.S. Pat. No. 7,034,144 (“Molecular detection of chromosome aberrations”); U.S. Pat. No. 7,014,997 (“Chromosome structural abnormality localization with single copy probes”); U.S. Pat. No. 6,677,123 (“Process for detecting increased risk of fetal chromosomal abnormality”); U.S. Pat. No. 6,607,877 (“Methods and compositions for chromosome-specific staining”); U.S. Pat. No. 6,566,069 (“Gene sequencer and method for determining the nucleotide sequence of a chromosome”); U.S. Pat. No. 6,455,258 (“Detection of chromosome copy number changes to distinguish melanocytic nevi from malignant melanoma”); U.S. Pat. No. 6,344,315 (“Chromosome-specific staining to detect genetic rearrangements associated with chromosome 3 and/or chromosome 17”); U.S. Pat. No. 6,280,929 (“Method of detecting genetic translocations identified with chromosomal abnormalities”); U.S. Pat. No. 6,277,569 (“Methods for multiple direct label probe detection of multiple chromosomes or regions thereof by in situ hybridization”).

Sevently, some variants may include special-purpose circuitry for characterizing cell and/or organ types or otherwise processing device-detectable data. In light of teachings herein, numerous existing techniques may be applied for implementing such modules 2853 of evaluation logic 2850 as described herein without undue experimentation. See, e.g., U.S. Pat. No. 7,289,835 (“Multivariate analysis of green to ultraviolet spectra of cell and tissue samples”); U.S. Pat. No. 7,277,740 (“Analysis system for reagent-free determination of the concentration of an analyte in living tissue”); U.S. Pat. No. 7,233,330 (“Organ wall analysis with ray-casting”); U.S. Pat. No. 7,167,734 (“Method for optical measurements of tissue to determine disease state or concentration of an analyte”); U.S. Pat. No. 7,155,050 (“Method of analyzing cell samples, by creating and analyzing a resultant image”); U.S. Pat. No. 7,050,842 (“Method of tissue modulation for noninvasive measurement of an analyte”); U.S. Pat. No. 6,716,633 (“Blood cell detector, blood analyzer and blood analyzing method using the detector”); U.S. Pat. No. 46,461,828 (“Conjunctive analysis of biological marker expression for diagnosing organ failure”); U.S. Pat. No. 6,372,183 (“Automated analysis equipment and assay method for detecting cell surface protein and/or cytoplasmic receptor function using same”); U.S. Pat. No. 6,174,698 (“Micro lysis-analysis process to measure cell characteristics”); U.S. Pat. No. 6,080,551 (“Rapid assays for the assessment of organ status based on the detection of one or more isoenzymes of glutathione S-transferase”).

Eighthly, some variants may include special-purpose circuitry for processing data from one or more assays. In light of teachings herein, numerous existing techniques may be applied for implementing such modules 2856 of evaluation logic 2850 as described herein without undue experimentation. See, e.g., U.S. Pat. No. 7,351,546 (“Flow cytometric, whole blood dendritic cell immune function assay”); U.S. Pat. No. 7,230,086 (“Assay for YKL-40 as a marker for degradation of mammalian connective tissue matrices”); U.S. Pat. No. 7,226,753 (“Displacement assay for selective biological material detection”); U.S. Pat. No. 7,217,564 (“Cytotoxic assay and new established cell line of sturgeon origin”); U.S. Pat. No. 7,214,505 (“Cell-based assay for the detection of toxic analytes”); U.S. Pat. No. 7,045,311 (“Whole cell assay systems for cell surface proteases”); U.S. Pat. No. 6,864,053 (“Quantitative assay of host cell DNA in a sample”); U.S. Pat. No. 6,852,906 (“Assay for measuring enzyme activity in vivo”); U.S. Pat. No. 6,849,406 (“Reverse transcriptase assay kit, use thereof and method for analysis of RT activity in biological samples”); U.S. Pat. No. 6,790,611 (“Assay for directly detecting a RS virus related biological cell in a body fluid sample”); U.S. Pat. No. 6,756,233 (“Method for measuring free ligands in biological fluids, and assay kits for measuring same”); U.S. Pat. No. 6,610,494 (“Solid-phase activity assay for biologically active substance”); U.S. Pat. No. 6,455,684 (“Insitu assay of substance in biological sample using labeled probe”); U.S. Pat. No. 6,391,555 (“Assay for the detection of avian leukosis/sarcoma viruses (ALSV) in DNA from human and animal biological specimens”); U.S. Pat. No. 6,372,183 (“Automated analysis equipment and assay method for detecting cell surface protein and/or cytoplasmic receptor function using same”); U.S. Pat. No. 6,159,699 (“Enzyme linked chemiluminescent assay”). Some such variants, for example, may include pattern recognition logic 1150 or other circuitry for processing image data 871, evaluation data 1180, video data 1686, sensor data, result data 2070, digital output, or other data 1279, 2441 as described herein. Alternatively or additionally, such modules may implement or otherwise interact with one or more protocols 2483, 2493 relating to an assay as described herein.

Ninthly, some variants may categorically or otherwise indicate one or more cellular specializations, orientations, response characteristics, morphologies 1032, biomarkers 1075, or other cell attributes. In light of teachings herein, numerous existing techniques may be applied for relating such output from one or more modules 2857 of evaluation logic 2850 to such attributes as described herein without undue experimentation. See, e.g., U.S. Pat. No. 7,384,781 (“Sensors for biomolecular detection and cell classification”); U.S. Pat. No. 7,183,389 (“Monoclonal antibodies and cell surface antigens for the detection and treatment of small cell lung cancer (SCLC)”); U.S. Pat. No. 7,045,311 (“Whole cell assay systems for cell surface proteases”); U.S. Pat. No. 6,975,899 (“Multi-modal optical tissue diagnostic system”); U.S. Pat. No. 6,927,049 (“Cell viability detection using electrical measurements”); U.S. Pat. No. 6,670,197 (“Method for assaying whole blood for the presence or absence of circulating cancer or other target cell fragments”); U.S. Pat. No. 6,599,694 (“Method of characterizing potential therapeutics by determining cell-cell interactions”); U.S. Pat. No. 6,123,860 (“Method for separating cell populations by thermophilic characteristics”); U.S. Pat. No. 6,106,778 (“Blood cell count/immunoassay apparatus using whole blood”); U.S. Pat. No. 7,387,895 (“Monoclonal antibody specific for PPAR gamma, hydridoma cell line producing the same, and method for detecting regulator related to diseases, including inflammation, cancer and metabolic diseases, using the same”); U.S. Pat. No. 7,354,775 (“Reagent for partially lysing a cell membrane of a red blood cell, a reagent for detecting malaria infected red blood cells, and a sample analyzing method for detecting malaria infected red blood cells”); U.S. Pat. No. 7,291,710 (“Detection of spectrin and spectrin proteolytic cleavage products in assessing nerve cell damage”); U.S. Pat. No. 7,256,252 (“Methods for detecting cell apoptosis”); U.S. Pat. No. 7,166,427 (“Detecting the expression of the DESC l gene in squamous cell carcinoma”); U.S. Pat. No. 7,155,361 (“Semiconductor test management system and method”); U.S. Pat. No. 7,155,050 (“Method of analyzing cell samples, by creating and analyzing a resultant image”); U.S. Pat. No. 7,112,415 (“Method of preparing cell cultures from biological specimens for assaying a response to an agent”); U.S. Pat. No. 7,105,292 (“Screening methods used to identify compounds that modulate a response of a cell to ultraviolet radiation exposure”); U.S. Pat. No. 7,022,516 (“Well unit for detecting cell chemotaxis and separating chemotactic cells”); U.S. Pat. No. 6,958,221 (“Cell flow apparatus and method for real-time measurements of patient cellular responses”); U.S. Pat. No. 6,900,049 (“Adenovirus vectors containing cell status-specific response elements and methods of use thereof”); U.S. Pat. No. 6,808,890 (“Method of detecting a cancerous cell expressing EGFL6, and EGF mutif protein”); U.S. Pat. No. 6,607,879 (“Compositions for the detection of blood cell and immunological response gene expression”); U.S. Pat. No. 6,372,183 (“Automated analysis equipment and assay method for detecting cell surface protein and/or cytoplasmic receptor function using same”). Some such variants, for example, may include pattern recognition logic 1150 or other circuitry for processing image data 871, video data 1686, sensor data, evaluation data 1180, result data 2070, digital output, or other data 1279, 2441 as described herein. Some such modules, for example, may include software-controlled or other circuitry for processing device-detectable data obtained from one or more biomarker detection protocols as described herein. Alternatively or additionally, such modules may implement or otherwise interact with one or more protocols 2483, 2493 for evaluating data, for example, from an assay as described above.

Tenthly, some variants may include medical databases or other special-purpose circuitry for characterizing types of genetic/chromosomal abnormalities and their consequences. In light of teachings herein, numerous existing techniques may be applied for implementing such modules 2858 of evaluation logic 2850 as described herein without undue experimentation. See, e.g., U.S. Pat. No. 7,371,522 (“Use of polymorphism of the serotonin transporter gene promoter as a predictor of disease risk”); U.S. Pat. No. 7,217,547 (“Aspartoacylase gene, protein, and methods of screening for mutations associated with Canavan disease”); U.S. Pat. No. 7,141,373 (“Method of haplotype-based genetic analysis for determining risk for developing insulin resistance and coronary artery disease”); U.S. Pat. No. 7,094,534 (“Detection of chromosoal abnormalities associated with breast cancer”); U.S. Pat. No. 7,060,438 (“Method for analyzing a patient's genetic prediposition to at least one disease and amplification adapted to such a method”); U.S. Pat. No. 6,973,388 (“Methods of diagnosing disease states using gene expression profiles”); U.S. Pat. No. 6,808,881 (“Method for determining susceptibility to heart disease by screening polymorphisms in the vitamin D receptor gene”); U.S. Pat. No. 6,673,546 (“Genetic loci indicative of propensity for longevity and methods for identifying propensity for age-related disease”); U.S. Pat. No. 6,485,911 (“Methods for determining risk of developing alzheimer's disease by detecting mutations in the presenilin 2 (PS-2) gene”); U.S. Pat. No. 6,306,603 (“CD36 mutant gene and methods for diagnosing diseases caused by abnormal lipid metabolism and diagnostic kits therefor”); U.S. Pat. No. 6,280,929 (“Method of detecting genetic translocations identified with chromosomal abnormalities”); U.S. Pat. No. 6,251,601 (“Simultaneous measurement of gene expression and genomic abnormalities using nucleic acid microarrays”); U.S. Pat. No. 6,225,069 (“Methods to identify genetic predisposition to alzheimer's disease”); U.S. Pat. No. 6,221,607 (“Automated fluorescence in situ hybridization detection of genetic abnormalities”); U.S. Pat. No. 6,210,889 (“Method for enrichment of fetal cells from maternal blood and use of same in determination of fetal sex and detection of chromosomal abnormalities”).

Alternatively or additionally, some variants may measure, image, or otherwise indicate an organ or other cell group's attributes. In light of teachings herein, numerous existing techniques may be applied for relating such output from one or more modules 2854 of evaluation logic 2850 to such attributes as described herein without undue experimentation. See, e.g., U.S. Pat. No. 7,333,845 (“Non-invasive imaging for determination of global tissue characteristics”); U.S. Pat. No. 7,309,867 (“Methods and apparatus for characterization of tissue samples”); U.S. Pat. No. 7,301,629 (“Apparatus and method for determining tissue characteristics”); U.S. Pat. No. 7,257,244 (“Elastography imaging modalities for characterizing properties of tissue”); U.S. Pat. No. 7,155,042 (“Method and system of measuring characteristics of an organ”); U.S. Pat. No. 7,074,188 (“System and method of characterizing vascular tissue”); U.S. Pat. No. 7,004,902 (“Method and apparatus for measuring biomechanical characteristics of corneal tissue”); U.S. Pat. No. 6,975,899 (“Multi-modal optical tissue diagnostic system”); U.S. Pat. No. 6,954,667 (“Method for Raman chemical imaging and characterization of calcification in tissue”); U.S. Pat. No. 6,912,412 (“System and methods of fluorescence, reflectance and light scattering spectroscopy for measuring tissue characteristics”); U.S. Pat. No. 6,678,552 (“Tissue characterization based on impedance images and on impedance measurements”); U.S. Pat. No. 6,507,747 (“Method and apparatus for concomitant structural and biochemical characterization of tissue”); U.S. Pat. No. 6,208,749 (“Systems and methods for the multispectral imaging and characterization of skin tissue”); U.S. Pat. No. 6,024,698 (“Apparatus for monitoring functional characteristics of an organ intended for transplantations”); U.S. Pat. No. 7,372,985 (“Systems and methods for volumetric tissue scanning microscopy”); U.S. Pat. No. 7,366,365 (“Tissue scanning apparatus and method”); U.S. Pat. No. 7,359,548 (“Method and apparatus for automated image analysis of biological specimens”); U.S. Pat. No. 7,230,242 (“Methods for SEM inspection of fluid containing samples”); U.S. Pat. No. 7,129,473 (“Optical image pickup apparatus for imaging living body tissue”); U.S. Pat. No. 6,909,792 (“Historical comparison of breast tissue by image processing”); U.S. Pat. No. 6,594,021 (“Analysis system for interferometric scanning of donor corneal tissue”); U.S. Pat. No. 6,510,338 (“Method of and devices for fluorescence diagnosis of tissue, particularly by endoscopy”); U.S. Pat. No. 6,408,050 (“X-ray detector and method for tissue specific image”); U.S. Pat. No. 6,364,829 (“Autofluorescence imaging system for endoscopy”); U.S. Pat. No. 6,256,530 (“Optical instrument and technique for cancer diagnosis using in-vivo fluorescence emission of test tissue”); U.S. Pat. No. 6,165,128 (“Method and apparatus for making an image of a lumen or other body cavity and its surrounding tissue”).

Alternatively or additionally, some variants may include software-controlled or other special-purpose circuitry for selecting a dispenser 921, 1540 or otherwise causing at least a component of tissue to come into contact with a stain effective for indicating whether the tissue exhibits an abnormality in a chromosomal pattern 1040 or some other attribute of interest. In light of teachings herein, numerous existing techniques may be applied for relating such output from one or more modules 2862 of selection logic 2870 to such attributes as described herein without undue experimentation. See, e.g., U.S. Pat. No. 7,344,587 (“Magnetic ink tissue markings”); U.S. Pat. No. 7,332,360 (“Early detection of metal wiring reliability using a noise spectrum”); U.S. Pat. No. 7,329,414 (“Biodegradable polymer for marking tissue and sealing tracts”); U.S. Pat. No. 7,285,364 (“Permanent, removable tissue markings”); U.S. Pat. No. 7,047,063 (“Tissue site markers for in vivo imaging”); U.S. Pat. No. 7,015,013 (“Method for localized staining of an intact corneal tissue surface”); U.S. Pat. No. 6,998,270 (“Automated tissue staining system and reagent container”); U.S. Pat. No. 6,830,743 (“In vivo stain compounds and methods of use to identify dysplastic tissue”); U.S. Pat. No. 6,599,496 (“Endoscopy tissue stain”); U.S. Pat. No. 6,436,348 (“Staining apparatus for preparation of tissue specimens placed on microscope slides”); U.S. Pat. No. 6,086,852 (“In vivo stain composition, process of manufacture, and methods of use to identify dysplastic tissue”); U.S. Pat. No. 6,017,495 (“Staining apparatus for staining of tissue specimens on microscope slides”).

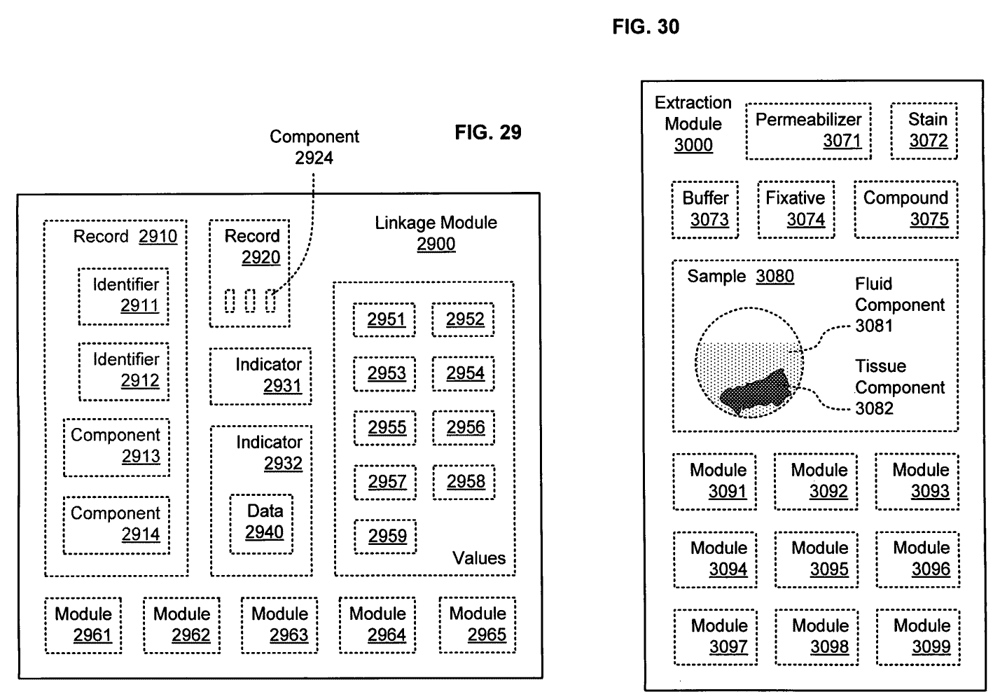

With reference now to FIG. 29, shown is a context in which one or more technologies may be implemented in a linking module 2900 (among two or more instruments, modules, networks, users, or other such resources, e.g.). In some variants, software-controlled or other modules 2961, 2962, 2963, 2964, 2965 of linking module 2900 may be configured to process or otherwise bear one or more records 2910, 2920; values 2951, 2952, 2953, 2954, 2955, 2956, 2957, 2958, 2959; identifiers 2911, 2912 or other components 2913, 2914, 2924; data 2940; or other indicators 2931, 2932 as described herein.

In some variants, such data “indicates” a therapeutic or other treatment of tissue or an extraction. This can occur, for example, in a context in which the treatment has an optical or other detectable effect upon some component of extracted matter. Alternatively or additionally, such an effect may be conditional upon a molecular structure being present in the tissue or extraction, for example, such that an absence of the detectable effect indicates a lower likelihood and/or concentration of the molecular structure.

Some variants may include special-purpose circuitry or other components for applying hybridization or other diagnostic protocols to one or more cells of a sample. In light of teachings herein, numerous existing techniques may be applied by one or more modules 2863, 2962 of selection or other logic for invoking an appropriate diagnostic protocol. Some such variants, for example, may include media bearing one or more excitation wavelengths, emission wavelengths, magnifications or other such values 2955, 2956 usable in a fluorescence microscope 2406 as described herein without undue experimentation. See, e.g., U.S. Pat. No. 7,348,361 (“Solution for diagnosing or treating tissue pathologies”); U.S. Pat. No. 7,326,575 (“Methods and compositions for the preparation and use of fixed-treated cell-lines and tissue in fluorescence in situ hybridization”); U.S. Pat. No. 7,237,392 (“System for preparing cutaneous tissue samples for oncological histology study and diagnosis”); U.S. Pat. No. 7,230,086 (“Assay for YKL-40 as a marker for degradation of mammalian connective tissue matrices”); U.S. Pat. No. 6,946,287 (“Device for providing a hybridization chamber, and process unit and system for hybridizing nucleic acid samples, proteins, and tissue sections”); U.S. Pat. No. 6,852,906 (“Assay for measuring enzyme activity in vivo”); U.S. Pat. No. 6,697,665 (“Systems and methods of molecular spectroscopy to provide for the diagnosis of tissue”); U.S. Pat. No. 6,510,338 (“Method of and devices for fluorescence diagnosis of tissue, particularly by endoscopy”); U.S. Pat. No. 6,296,608 (“Diagnosing and performing interventional procedures on tissue in vivo”); U.S. Pat. No. 6,159,699 (“Enzyme linked chemiluminescent assay”); U.S. Pat. No. 6,157,856 (“Tissue diagnostics using evanescent spectroscopy”); U.S. Pat. No. 5,998,139 (“Assay for determination of neuronal activity in brain tissue”). Alternatively or additionally, such modules or media may receive or otherwise obtain a diagnostic identifier or other result of positioning a cell in a microfluidic structure.

Some variants may include special-purpose modules 2964 or other circuitry for causing diagnostic procedures on body fluids or other sample components. In light of teachings herein, numerous existing techniques may be applied for obtaining a karyotype or other data component relating to tissue, blood, or other fluid extracted from an organism as described herein without undue experimentation. See, e.g., U.S. Pat. No. 7,384,791 (“Method of analyzing blood”); U.S. Pat. No. 7,354,775 (“Reagent for partially lysing a cell membrane of a red blood cell, a reagent for detecting malaria infected red blood cells, and a sample analyzing method for detecting malaria infected red blood cells”); U.S. Pat. No. 7,316,649 (“Method and apparatus for non-invasive analysis of blood glucose”); U.S. Pat. No. 7,276,376 (“Analyzing method of a blood coagulation reaction”); U.S. Pat. No. 7,258,673 (“Devices, systems and methods for extracting bodily fluid and monitoring an analyte therein”); U.S. Pat. No. 7,192,405 (“Integrated lancet and bodily fluid sensor”); U.S. Pat. No. 7,188,515 (“Nanoliter viscometer for analyzing blood plasma and other liquid samples”); U.S. Pat. No. 7,150,995 (“Methods and systems for point of care bodily fluid analysis”); U.S. Pat. No. 7,027,134 (“Spectrophotometric system and method for the identification and characterization of a particle in a bodily fluid”); U.S. Pat. No. 7,016,021 (“Method for measuring concentration of component contained in bodily fluid and apparatus for measuring concentration of component contained in bodily fluid”); U.S. Pat. No. 7,004,901 (“Method and kit for the transdermal determination of analyte concentration in blood”); U.S. Pat. No. 6,736,777 (“Biosensor, iontophoretic sampling system, and methods of use thereof”); U.S. Pat. No. 6,718,189 (“Method and apparatus for non-invasive blood analyte measurement with fluid compartment equilibration”); U.S. Pat. No. 6,339,722 (“Apparatus for the in-vivo non-invasive measurement of a biological parameter concerning a bodily fluid of a person or animal”); U.S. Pat. No. 6,246,785 (“Automated, microscope-assisted examination process of tissue or bodily fluid samples”); U.S. Pat. No. 6,023,639 (“Non-invasive bodily fluid withdrawal and monitoring system”); U.S. Pat. No. 5,569,225 (“Bodily fluid test kit and method of testing bodily fluids”). Some such variants, for example, may include media bearing one or more magnifications, capture modes, or other such values 2953, 2954 usable in a digital microscope 2270 as described herein. Alternatively or additionally, such modules may comprise or otherwise interact with media bearing one or more spectral ranges, acquisition durations, or other such values 2952, 2954 usable in a spectrometer 2420 as described herein.

In light of teachings herein, numerous existing techniques may be applied for configuring antibodies for detecting antigens of particular interest as described herein. Some variants may include special-purpose modules 2965 or other circuitry for detecting a result of antibody-containing or other optical enhancement materials indicating an absence of or a presence of a chromosomal pattern 1040 or other attribute in a cell, for example, without undue experimentation. See, e.g., U.S. Pat. No. 7,396,915 (“Monoclonal antibody and gene encoding the same, hybridoma, pharmaceutical composition, and diagnostic reagent”); U.S. Pat. No. 7,387,895 (“Monoclonal antibody specific for PPAR gamma, hydridoma cell line producing the same, and method for detecting regulator related to diseases, including inflammation, cancer and metabolic diseases, using the same”); U.S. Pat. No. 7,364,863 (“Monoclonal antibody W8B2 and method of use”); U.S. Pat. No. 7,320,791 (“Monoclonal antibody for analysis and clearance of polyethylene glycol and polyethylene glycol-modified molecules”); U.S. Pat. No. 7,241,578 (“Immunoassay method/equipment, biological component measurable toilet, anti-albumin monoclonal antibody, cell strain producing the same, and albumin detection kit”); U.S. Pat. No. 7,198,104 (“Subterranean fluids and methods of cementing in subterranean formations”); U.S. Pat. No. 7,148,332 (“High affinity monoclonal antibody for recognizing the estrogen receptor (ER) and method for creating the antibody”); U.S. Pat. No. 7,087,396 (“Monoclonal antibody and method and kit for immunoassay of soluble human ST2”); U.S. Pat. No. 7,038,021 (“Anti-dioxins monoclonal antibody suitable for assaying dioxins in environment and hybridoma producing the same”); U.S. Pat. No. 6,989,241 (“Assay for rapid detection of human activated protein C and highly specific monoclonal antibody therefor”); U.S. Pat. No. 6,919,435 (“Human lung adenocarcinoma-related monoclonal antibody and antigen and immunoassay method which uses the same”); U.S. Pat. No. 6,849,419 (“Monoclonal antibody hybridoma immunoassay method and diagnosis kit”); U.S. Pat. No. 6,787,153 (“Human monoclonal antibody specifically binding to surface antigen of cancer cell membrane”); U.S. Pat. No. 6,709,833 (“Monoclonal antibody recognizing phosphatidylinositol-3,4-diphosphate”). Alternatively or additionally, such modules may comprise or otherwise interact with media bearing one or more excitation wavelengths, emission wavelengths, magnifications or other such values 2955, 2956 usable in a fluorescence microscope 2406 as described herein.

Some variants may include special-purpose circuitry for including or otherwise interacting with protein-based arrays, biopolymers, or other biosensors (of probes 210, 1510 or other instruments 1110, e.g.). This can occur, for example, in a context in which a conduit 1130 or other medium bears one or more biosensor-generated signals or other device-detectable data. In light of teachings herein, numerous existing techniques may be applied for implementing such modules 1154 of pattern recognition logic 1150 or other components as described herein without undue experimentation. See, e.g., U.S. Pat. No. 7,402,381 (“Method of immobilizing molecules onto a solid phase substrate and method of fabricating a biosensor using the method”); U.S. Pat. No. 7,323,347 (“Biosensor surface structures and methods”); U.S. Pat. No. 7,244,582 (“Immobilized carbohydrate biosensor”); U.S. Pat. No. 7,223,330 (“Biosensor, biosensor array and method for detecting macromolecular biopolymers with a biosensor”); U.S. Pat. No. 7,176,345 (“Transgenic animals expressing light-emitting fusion proteins and diagnostic and therapeutic methods therefor”); U.S. Pat. No. 6,977,160 (“Sensor protein and use thereof”); U.S. Pat. No. 6,960,466 (“Composite membrane containing a cross-linked enzyme matrix for a biosensor”); U.S. Pat. No. 6,783,958 (“Method of producing a biosensor protein capable of regulating a fluorescence property of green fluorescent protein, and the biosensor protein produced by the method”); U.S. Pat. No. 6,376,257 (“Detection by fret changes of ligand binding by GFP fusion proteins”); U.S. Pat. No. 5,965,713 (“Dye labeled protein conjugate its preparing method and sensor using the same”). Some such variants, for example, may include one or more protocol descriptors relating to a cell as described herein. Alternatively or additionally, such modules may include media bearing one or more resource addresses, invocation parameters, or other such values 2956, 2958 usable in a module 2454 of invocation logic 2455 as described herein.

Some variants may include special-purpose modules 1155 of pattern recognition logic 1150 or other circuitry for imaging and evaluating cells or other attributes of tissue. In light of teachings herein, numerous existing techniques may be applied writing or otherwise causing media to bear optical wavelengths, scan area coordinates, or other such values 2951, 2952 usable in laser scanning equipment 2890 as described herein. Alternatively or additionally, such media may include one or more dispenser identifiers or other values indicative of contrast agents, pulse sequence or type descriptors, or other such values 2954, 2955 usable in MRI scanners 2402, ultrasound imaging equipment, or other such devices. See, e.g., U.S. Pat. No. 7,155,050 (“Method of analyzing cell samples, by creating and analyzing a resultant image”); U.S. Pat. No. 7,129,473 (“Optical image pickup apparatus for imaging living body tissue”); U.S. Pat. No. 6,900,009 (“Method for creating a frozen tissue array”); U.S. Pat. No. 6,893,837 (“Frozen tissue microarray technology for analysis RNA, DNA, and proteins”); U.S. Pat. No. 6,811,766 (“Ultrasound imaging with contrast agent targeted to microvasculature and a vasodilator drug”); U.S. Pat. No. 6,544,794 (“Method for visual imaging of ion distribution in tissue”); U.S. Pat. No. 6,463,438 (“Neural network for cell image analysis for identification of abnormal cells”); U.S. Pat. No. 6,408,050 (“X-ray detector and method for tissue specific image”); U.S. Pat. No. 6,032,068 (“Non-invasive measurement of frozen tissue temperature using MRI signal”); U.S. Pat. No. 5,854,851 (“System and method for diagnosis of living tissue diseases using digital image processing”); U.S. Pat. No. 5,741,648 (“Cell analysis method using quantitative fluorescence image analysis”); U.S. Pat. No. 5,024,830 (“Method for cryopreparing biological tissue for ultrastructural analysis”). Some such variants, for example, may include one or more protocols for treating, imaging, evaluating, and/or extracting cells that are or will be frozen. Alternatively or additionally, such modules may comprise or otherwise interact with images depicting cellular or other features from electron microscopes 770, image recognition modules 1152, fluorescence microscopes 2406, video microscopes 2408, or other image-handling equipment as described herein.

Some variants may include one or more statistical evaluations or other quantifications characterizing an image or other optical field of a sensor, extraction, or other mode or region. In light of teachings herein, numerous existing techniques may be applied for relating such output from one or more modules 1622 of evaluation logic 1620 to such attributes as described herein without undue experimentation. See, e.g., U.S. Pat. No. 7,416,531 (“System and method of detecting and processing physiological sounds”); U.S. Pat. No. 7,397,545 (“Application of statistical inference to optical time domain reflectometer data”); U.S. Pat. No. 7,330,588 (“Image metrics in the statistical analysis of DNA microarray data”); U.S. Pat. No. 7,310,590 (“Time series anomaly detection using multiple statistical models”); U.S. Pat. No. 7,248,921 (“Method and devices for performing cardiac waveform appraisal”); U.S. Pat. No. 7,190,394 (“Method for statistical analysis of images for automatic white balance of color channel gains for image sensors”); U.S. Pat. No. 7,155,050 (“Method of analyzing cell samples, by creating and analyzing a resultant image”); U.S. Pat. No. 7,082,224 (“Statistic calculating method using a template and corresponding sub-image to determine similarity based on sum of squares thresholding”); U.S. Pat. No. 7,016,786 (“Statistical methods for analyzing biological sequences”); U.S. Pat. No. 6,804,394 (“System for capturing and using expert's knowledge for image processing”); U.S. Pat. No. 6,718,068 (“Noise reduction method utilizing statistical weighting, apparatus, and program for digital image processing”); U.S. Pat. No. 6,507,633 (“Method for statistically reconstructing a polyenergetic X-ray computed tomography image and image reconstructor apparatus utilizing the method”); U.S. Pat. No. 6,161,089 (“Multi-subframe quantization of spectral parameters”). Alternatively or additionally, such modules may include or otherwise interact with media 100, 1000 bearing one or more feature definitions, ranges, shape types, or other such values 2955, 2958 usable in one or more modules 1155 of image or other pattern recognition logic 1150 as described herein.

In some variants, the above-described systems and methods may incorporate or otherwise operate in conjunction with an adhesive or other mode of fixation and/or extraction. One or more parametric values 2957, 2959 relating to such variants may determine or otherwise indicate one or more of a contact time, an energy transfer rate, a ratio of ingredients, a penetration or other engagement force, an agent or component selection, an amount of tissue extracted, or other such quantities.

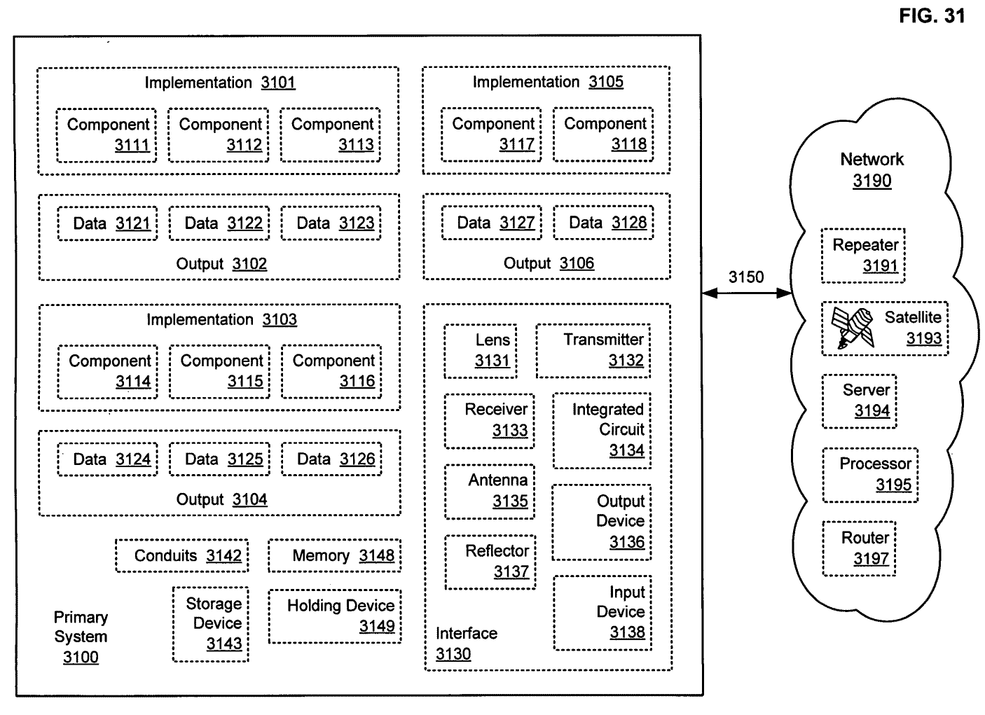

With reference now to FIG. 30, shown is a context in which one or more technologies may be implemented. An extraction module 3000 may include one or more permeabilizers 3071, stains 3072, buffers 3073, fixatives 3074, or other components in compounds 3075 configured to treat one or more samples 3080. Such samples or other extractions may include one or more solid or semi-solid tissue components 3082, for example, as well as (sputum, sap, interstitial fluid, cytoplasm, or other) fluid components 3081. Alternatively or additionally, such extraction modules may include one or more modules 3091, 3092, 3093, 3094, 3095, 3096, 3097, 3098, 3099 for controlling dispensations, extractions, evaluations, or other such protocols as described below.

Some variants may include or otherwise interact with one or more modules and/or protocols for configuring a frozen or other tissue sample for shipment or long-term storage. In light of teachings herein, numerous existing techniques may be applied for configuring one or more modules 2845 of control logic 2840 to implement such features as described herein without undue experimentation. See, e.g., U.S. Pat. No. 7,371,513 (“Method of preserving corneal tissue using polyoxyethylene/polyoxypropylene copolymer”); U.S. Pat. No. 7,129,035 (“Method of preserving tissue”); U.S. Pat. No. 7,014,990 (“Machine perfusion solution for organ and biological tissue preservation”); U.S. Pat. No. 7,005,253 (“Cold storage solution for organ and biological tissue preservation”); U.S. Pat. No. 6,994,954 (“System for organ and tissue preservation and hypothermic blood substitution”); U.S. Pat. No. 6,946,241 (“Physiological medium for perfusing, preserving and storing isolated cell, tissue and organ samples”); U.S. Pat. No. 6,942,961 (“Method for dehydrating biological tissue for producing preserved transplants”); U.S. Pat. No. 6,569,615 (“Composition and methods for tissue preservation”); U.S. Pat. No. 6,492,103 (“System for organ and tissue preservation and hypothermic blood substitution”); U.S. Pat. No. 6,270,986 (“Method of preserving biological tissue specimens and method of infrared spectroscopic analysis which avoids the effects of polymorphs”); U.S. Pat. No. 6,207,658 (“Preservation of tissue during removal storage and implantation”); U.S. Pat. No. 5,964,096 (“Method and package design for cryopreservation and storage of cultured tissue equivalents”). Alternatively or additionally, such modules may comprise or otherwise interact with pattern recognition logic 1150, evaluation logic 2850, or other circuitry for processing data and/or samples 1112, 2062, 3080 as described herein.

Alternatively or additionally, some variants may implement protocols for configuring a molecular probe or other biosensor to detect an effect, pH, density, concentration, structure, constitution, or other attribute of a fluid component 3081 or other form of matter. In light of teachings herein, numerous existing techniques may be applied for implementing one or more control modules 3094 for such functions as described herein without undue experimentation. See, e.g., U.S. Pat. No. 7,396,687 (“Mass spectrometric immunoassay analysis of specific proteins and variants present in various biological fluids”); U.S. Pat. No. 7,359,743 (“System for monitoring and calculating integrated tissue pH”); U.S. Pat. No. 7,359,548 (“Method and apparatus for automated image analysis of biological specimens”); U.S. Pat. No. 7,323,347 (“Biosensor surface structures and methods”); U.S. Pat. No. 7,319,046 (“Integrated optoelectronic silicon biosensor for the detection of biomolecules labeled with chromophore groups or nanoparticles”); U.S. Pat. No. 7,191,068 (“Proteomic analysis of biological fluids”); U.S. Pat. No. 7,112,433 (“Electrical analysis of biological membranes”); U.S. Pat. No. 7,033,321 (“Ultrasonic water content monitor and methods for monitoring tissue hydration”); U.S. Pat. No. 6,979,728 (“Articles of manufacture and methods for array based analysis of biological molecules”); U.S. Pat. No. 6,913,697 (“Nanostructured separation and analysis devices for biological membranes”); U.S. Pat. No. 6,790,669 (“Method for chemical analysis of biological material”); U.S. Pat. No. 6,600,941 (“Systems and methods of pH tissue monitoring”); U.S. Pat. No. 6,479,019 (“Sensor and sensor assembly for detecting a target gas in a breath sample”); U.S. Pat. No. 6,372,183 (“Automated analysis equipment and assay method for detecting cell surface protein and/or cytoplasmic receptor function using same”); U.S. Pat. No. 5,965,713 (“Dye labeled protein conjugate its preparing method and sensor using the same”).

Alternatively or additionally, some variants may include special-purpose modules or other circuitry for generating and/or evaluating images, measurements, or other data from minimally invasive or noninvasive protocols. In light of teachings herein, numerous existing techniques may be applied for implementing such detection modules 3098 as described herein without undue experimentation. See, e.g., U.S. Pat. No. 7,415,146 (“Method and apparatus to determine bone mineral density utilizing a flat panel detector”); U.S. Pat. No. 7,415,139 (“Living-tissue pattern detecting method, living-tissue pattern detecting device, biometric authentication method, and biometric authentication device”); U.S. Pat. No. 7,409,040 (“System and method for noninvasive diagnostic imaging, detection, and identification of substances by microwave/RF modulation of x-rays and applications in treatment of diseases characterized by the presence of pathological macromolecules or by the need for regeneration of normal tissue”); U.S. Pat. No. 7,261,693 (“Soft tissue diagnostic apparatus and method”); U.S. Pat. No. 7,133,717 (“Tissue electroperforation for enhanced drug delivery and diagnostic sampling”); U.S. Pat. No. 7,043287 (“Method for modulating light penetration depth in tissue and diagnostic applications using same”); U.S. Pat. No. 6,975,899 (“Multi-modal optical tissue diagnostic system”); U.S. Pat. No. 6,697,665 (“Systems and methods of molecular spectroscopy to provide for the diagnosis of tissue”); U.S. Pat. No. 6,510,338 (“Method of and devices for fluorescence diagnosis of tissue, particularly by endoscopy”); U.S. Pat. No. 6,507,748 (“Compression apparatus for diagnostically examining breast tissue”); U.S. Pat. No. 6,505,079 (“Electrical stimulation of tissue for therapeutic and diagnostic purposes”); U.S. Pat. No. 6,174,291 (“Optical biopsy system and methods for tissue diagnosis”); U.S. Pat. No. 6,045,511 (“Device and evaluation procedure for the depth-selective, noninvasive detection of the blood flow and/or intra and/or extra-corporeally flowing liquids in biological tissue”).

Alternatively or additionally, some such variants, for example, may include frozen or other superficial extractions as described herein. Alternatively or additionally, such modules may comprise or otherwise interact with probes configured to transmit a signal arising from a hybridization protocol or other mode of analyzing cells or other structures.

Alternatively or additionally, some variants may include software-controlled or other special-purpose circuitry for controlling a dispenser or otherwise causing a chemical or other treatment in vivo. In light of teachings herein, numerous existing techniques may be applied for implementing such control modules 3091 as described herein without undue experimentation. See, e.g., U.S. Pat. No. 7,371,744 (“Biologically active methylene blue derivatives”); U.S. Pat. No. 7,270,661 (“Electrosurgical apparatus and methods for treatment and removal of tissue”); U.S. Pat. No. 7,157,080 (“Injectable hyaluronic acid derivative with pharmaceuticals/cells”); U.S. Pat. No. 6,975,899 (“Multi-modal optical tissue diagnostic system”); U.S. Pat. No. 6,905,475 (“Method of injecting a drug and echogenic bubbles into prostate tissue”); U.S. Pat. No. 6,830,743 (“In Vivo stain compounds and methods of use to identify dysplastic tissue”); U.S. Pat. No. 6,699,294 (“Injectable implants for tissue augmentation and restoration”); U.S. Pat. No. 6,591,129 (“Method for treating tissue through injection of a therapeutic agent”); U.S. Pat. No. 6,586,407 (“Injectable pharmaceutical formulations for partricin derivatives”); U.S. Pat. No. 6,372,451 (“Histochemical labeling stain for myelin in brain tissue”); U.S. Pat. No. 6,368,637 (“Method and composition for topical treatment of viral lesions”); U.S. Pat. No. 6,296,608 (“Diagnosing and performing interventional procedures on tissue in vivo”); U.S. Pat. No. 6,083,487 (“Methylene blue and toluidene blue mediated fluorescence diagnosis of cancer”); U.S. Pat. No. 5,854,240 (“Methylene blue for the treatment or prophylaxis of encephalopathy caused by ifosfamide”); U.S. Pat. No. 5,827,217 (“Process and apparatus for harvesting tissue for processing tissue and process and apparatus for re-injecting processed tissue”); U.S. Pat. No. 5,308,772 (“Method for classifying and counting leukocytes”); U.S. Pat. No. 4,950,665 (“Phototherapy using methylene blue”).

Alternatively or additionally, some variants may include or otherwise interact with one or more extraction modules and/or protocols for preserving at least some structural aspects of a tissue sample 3080 or other specimen. In light of teachings herein, numerous existing techniques may be applied for configuring software-implemented or other control modules 3092 or other components to implement such features as described herein without undue experimentation. See, e.g., U.S. Pat. No. 7,371,513 (“Method of preserving corneal tissue using polyoxyethylene/polyoxypropylene copolymer”); U.S. Pat. No. 7,229,820 (“Apparatus and method for culturing and preserving tissue constructs”); U.S. Pat. No. 7,129,035 (“Method of preserving tissue”); U.S. Pat. No. 7,056,673 (“Preservation of RNA in a biological sample”); U.S. Pat. No. 7,014,990 (“Machine perfusion solution for organ and biological tissue preservation”); U.S. Pat. No. 7,005,253 (“Cold storage solution for organ and biological tissue preservation”); U.S. Pat. No. 6,962,774 (“Method for dry-preserving multicellular organism tissue at ordinary temperatures”); U.S. Pat. No. 6,946,241 (“Physiological medium for perfusing, preserving and storing isolated cell, tissue and organ samples”); U.S. Pat. No. 6,942,961 (“Method for dehydrating biological tissue for producing preserved transplants”); U.S. Pat. No. 6,881,543 (“Sampling and storage system for genetic material from tissue”); U.S. Pat. No. 6,746,711 (“Polymers with biocidal action, process for their preparation and their use”); U.S. Pat. No. 6,508,013 (“Method of quickly drying a fresh sample and method of preserving a dried body”); U.S. Pat. No. 6,458,762 (“Therapeutic use of hemoglobin for preserving tissue viability and reducing restenosis”); U.S. Pat. No. 6,283,228 (“Method for preserving core sample integrity”); U.S. Pat. No. 6,270,986 (“Method of preserving biological tissue specimens and method of infrared spectroscopic analysis which avoids the effects of polymorphs”); U.S. Pat. No. 6,207,658 (“Preservation of tissue during removal storage and implantation”); U.S. Pat. No. 5,341,692 (“Device for taking, preserving and transporting a fluid sample for analysis”).

Alternatively or additionally, some variants may include or otherwise interact with one or more extraction modules and/or protocols for drawing or otherwise manipulating a component of an organism's tissue. In light of teachings herein, numerous existing techniques may be applied for configuring software-implemented or other control modules 3093 or other components to implement such features as described herein without undue experimentation. See, e.g., U.S. Pat. No. 7,405,056 (“Tissue punch and tissue sample labeling methods and devices for microarray preparation, archiving and documentation”); U.S. Pat. No. 7,357,081 (“Safety and arming unit for a spinning projectile fuze”); U.S. Pat. No. 7,329,227 (“Forward-fired automatic tissue sampling apparatus with safety lock”); U.S. Pat. No. 7,270,661 (“Electrosurgical apparatus and methods for treatment and removal of tissue”); U.S. Pat. No. 7,241,874 (“Rapid isolation of osteoinductive protein mixtures from mammalian bone tissue”); U.S. Pat. No. 7,232,414 (“System and method for capturing body tissue samples”); U.S. Pat. No. 7,087,028 (“Method and apparatus for sampling cervical tissue”); U.S. Pat. No. 7,008,381 (“Device for taking a tissue sample”); U.S. Pat. No. 6,860,860 (“Tissue sampling and removal apparatus and method”); U.S. Pat. No. 6,641,575 (“Surgical vacuum instrument for retracting, extracting, and manipulating tissue”); U.S. Pat. No. 6,443,902 (“Ultrasound probe with a detachable needle guide, for collecting tissue samples”); U.S. Pat. No. 6,083,169 (“Method and an apparatus for the insertion of a needle guide into a patient in order to remove tissue samples”).

Alternatively or additionally, some variants may include software-controlled or other special-purpose circuitry for configuring or otherwise controlling laparoscopic instruments or other chambers adjacent tissue. In light of teachings herein, numerous existing techniques may be applied for implementing such control modules 3095 as described herein without undue experimentation. See, e.g., U.S. Pat. No. 7,405,056 (“Tissue punch and tissue sample labeling methods and devices for microarray preparation, archiving and documentation”); U.S. Pat. No. 7,329,227 (“Forward-fired automatic tissue sampling apparatus with safety lock”); U.S. Pat. No. 7,008,381 (“Device for taking a tissue sample”); U.S. Pat. No. 6,440,061 (“Laparoscopic instrument system for real-time biliary exploration and stone removal”); U.S. Pat. No. 6,383,195 (“Laparoscopic specimen removal apparatus”); U.S. Pat. No. 6,206,889 (“Device for removing anatomical parts by laparoscopy”); U.S. Pat. No. 5,713,368 (“Single use automated soft tissue aspiration biopsy device”); U.S. Pat. No. 5,451,524 (“In vitro chamber for human organ tissue samples”).

Alternatively or additionally, some variants may include software-controlled or other special-purpose circuitry for controlling an emitter 531, 1642 in vitro or otherwise administering a treatment with an optical component. In light of teachings herein, numerous existing techniques may be applied for implementing such control modules 3096 as described herein without undue experimentation. See, e.g., U.S. Pat. No. 7,411,672 (“Method and apparatus for chemical imaging in a microfluidic circuit”); U.S. Pat. No. 7,351,252 (“Method and apparatus for photothermal treatment of tissue at depth”); U.S. Pat. No. 7,328,060 (“Cancer detection and adaptive dose optimization treatment system”); U.S. Pat. No. 7,288,106 (“System and method for excitation of photoreactive compounds in eye tissue”); U.S. Pat. No. 7,252,815 (“Pathological tissue detection and treatment employing targeted benzoindole optical agents”); U.S. Pat. No. 7,220,256 (“Laser system and method for treatment of biological tissues”); U.S. Pat. No. 7,201,767 (“Device for ultraviolet radiation treatment of body tissues”); U.S. Pat. No. 6,394,964 (“Optical forceps system and method of diagnosing and treating tissue”); U.S. Pat. No. 5,454,807 (“Medical treatment of deeply seated tissue using optical radiation”).

Alternatively or additionally, some variants may include special-purpose circuitry for implementing various modes of imaging suitable for surgical applications. In light of teachings herein, numerous existing techniques may be applied for implementing such modules 3097 as described herein without undue experimentation. See, e.g., U.S. Pat. No. 7,130,676 (“Fluoroscopic image guided orthopaedic surgery system with intraoperative registration”); U.S. Pat. No. 7,072,704 (“System for indicating the position of a surgical probe within a head on an image of the head”); U.S. Pat. No. 6,763,259 (“Surgical system supported by optical coherence tomography”); U.S. Pat. No. 6,714,729 (“Automatic motion-controlled photographing apparatus and related photographing method”); U.S. Pat. No. 6,584,339 (“Method and apparatus for collecting and processing physical space data for use while performing image-guided surgery”); U.S. Pat. No. 46,301,495 (“System and method for intra-operative, image-based, interactive verification of a pre-operative surgical plan”); U.S. Pat. No. 6,192,267 (“Endoscopic or fiberscopic imaging device using infrared fluorescence”); U.S. Pat. No. 6,055,446 (“Continuous lengths of oxide superconductors”); U.S. Pat. No. 6,004,314 (“Optical coherence tomography assisted surgical apparatus”). Alternatively or additionally, such variants may include media bearing one or more recording durations, magnifications, or other such values 2951, 2953 usable in a video microscope 2408 as described herein.

Alternatively or additionally, some variants may include special-purpose circuitry for controlling, configuring, enabling, triggering, or otherwise facilitating extractions or other manipulations of tissue. In light of teachings herein, numerous existing techniques may be applied for implementing such modules 3099 as described herein (in an extraction module 3000, e.g.) without undue experimentation. See, e.g., U.S. Pat. No. 7,329,227 (“Forward-fired automatic tissue sampling apparatus with safety lock”); U.S. Pat. No. 7,232,414 (“System and method for capturing body tissue samples”); U.S. Pat. No. 7,156,814 (“Apparatus and method for harvesting and handling tissue samples for biopsy analysis”); U.S. Pat. No. 7,133,717 (“Tissue electroperforation for enhanced drug delivery and diagnostic sampling”); U.S. Pat. No. 7,041,114 (“Surgical tool and method for extracting tissue from wall of an organ”); U.S. Pat. No. 7,008,381 (“Device for taking a tissue sample”); U.S. Pat. No. 6,928,139 (“Method and device for sampling tissue during a radiological examination”); U.S. Pat. No. 6,695,791 (“System and method for capturing body tissue samples”); U.S. Pat. No. 6,641,575 (“Surgical vacuum instrument for retracting, extracting, and manipulating tissue”); U.S. Pat. No. 6,509,187 (“Method and device for collection and preparation of tissue samples for molecular genetic diagnostics”); U.S. Pat. No. 6,443,902 (“Ultrasound probe with a detachable needle guide, for collecting tissue samples”); U.S. Pat. No. 6,432,111 (“Device for extraction of tissue or the like”); U.S. Pat. No. 6,273,861 (“Pneumatically actuated tissue sampling device”); U.S. Pat. No. 6,152,932 (“Device for extraction of tissue”); U.S. Pat. No. 6,036,658 (“Cervical tissue sampling device and method”); U.S. Pat. No. 5,993,399 (“Automated tissue sampling device”); U.S. Pat. No. 5,643,313 (“Laparoscopic tissue compressor and extractor”). Some such variants, for example, may include media bearing one or more identifiers 2912 or other components 2913, protocol descriptors, or other such values 2951, 2954 usable in a syringe, probe, biopsy device, or other extraction module 660, 850, 3000 as described herein. Alternatively or additionally, such media may indicate one or more speeds, thicknesses, or other such values 2955, 2957 usable, for example, in microtomes 2884, tissue sampling devices, or other surgical instruments.

In light of teachings herein, numerous existing techniques may likewise be applied for operating or otherwise configuring a surgical probe for safely removing a tumor or other mass as described herein without undue experimentation. See, e.g., U.S. Pat. No. 7,270,661 (“Electrosurgical apparatus and methods for treatment and removal of tissue”); U.S. Pat. No. 6,984,239 (“Thrombectomy and tissue removal method”); U.S. Pat. No. 6,764,493 (“Tissue removal using biocompatible materials”); U.S. Pat. No. 6,758,842 (“Medical instrument for removing tissue, bone cement or the like in the human or animal body”); U.S. Pat. No. 6,743,228 (“Devices and methods for tissue severing and removal”); U.S. Pat. No. 6,730,098 (“Tissue removal pen”); U.S. Pat. No. 6,698,433 (“System and method for bracketing and removing tissue”); U.S. Pat. No. 6,685,472 (“Tool for removing soft tissue growth around a dental implant”); U.S. Pat. No. 6,418,338 (“Method for detecting and surgically removing lymphoid tissue involved in tumor progression”); U.S. Pat. No. 6,383,194 (“Flexible ultrasonic surgical snare”); U.S. Pat. No. 6,231,578 (“Ultrasonic snare for excising tissue”); U.S. Pat. No. 5,846,513 (“Tumor localization and removal system using penetratable detection probe and removal instrument”); U.S. Pat. No. 5,447,510 (“Apparatus comprising an ultrasonic probe for removing biologic tissue”).

With reference now to FIG. 31, shown is an example of a system that may serve as a context for introducing one or more processes, systems or other articles described herein. Primary system 3100 may include one or more instances of implementations 3101, 3103, 3105 or outputs 3102, 3104, 3106 that may be held or transmitted by interfaces 3130, conduits 3142, storage devices 3143, memories 3148, or other holding devices 3149 or the like. In various embodiments as described herein, for example, one or more instances of implementation components 3111, 3112, 3113, 3114, 3115, 3116, 3117, 3118 or implementation output data 3121, 3122, 3123, 3124, 3125, 3126, 3127, 3128 may each be expressed in any aspect or combination of software, firmware, or hardware as signals, data, designs, logic, instructions, or the like. The interface(s) 3130 may include one or more instances of lenses 3131, transmitters 3132, receivers 3133, integrated circuits 3134, antennas 3135, output devices 3136, reflectors 3137, input devices 3138, or the like for handling data or communicating with local users or with network 3190 via linkage 3150, for example. Several variants of primary system 3100 are described below with reference to one or more instances of repeaters 3191, communication satellites 3193, servers 3194, processors 3195, routers 3197, or other elements of network 3190.

Those skilled in the art will recognize that some list items may also function as other list items. In the above-listed types of media, for example, some instances of interface(s) 3130 may include conduits 3142, or may also function as storage devices that are also holding devices 3149. One or more transmitters 3132 may likewise include input devices or bidirectional user interfaces, in many implementations of interface(s) 3130. Each such listed term should not be narrowed by any implication from other terms in the same list but should instead be understood in its broadest reasonable interpretation as understood by those skilled in the art.

Several variants described herein refer to device-detectable “implementations” such as one or more instances of computer-readable code, transistor or latch connectivity layouts or other geometric expressions of logical elements, firmware or software expressions of transfer functions implementing computational specifications, digital expressions of truth tables, or the like. Such instances can, in some implementations, include source code or other human-readable portions. Alternatively or additionally, functions of implementations described herein may constitute one or more device-detectable outputs such as decisions, manifestations, side effects, results, coding or other expressions, displayable images, data files, data associations, statistical correlations, streaming signals, intensity levels, frequencies or other measurable attributes, packets or other encoded expressions, or the like from invoking or monitoring the implementation as described herein.

Referring again to FIG. 25, flow 2500 may be performed by one or more instances of server 3194 remote from primary system 3100, for example, but operable to cause output device(s) 3136 to receive and present results via linkage 3150. Alternatively or additionally, device-detectable data 3122 may be borne by one or more instances of signal-bearing conduits 3142, holding devices 3149, integrated circuits 3134, or the like as described herein. Such data may optionally be configured for transmission by a semiconductor chip or other embodiment of integrated circuit 3134 that contains or is otherwise operatively coupled with antenna 3135 (in a radio-frequency identification tag, for example).

In some variants, some instances of flow 2500 may be implemented entirely within primary system 3100, optionally configured as a stand-alone system. Operation 2560 may be implemented by configuring one or more components 3111, 3112 as logic for obtaining device-detectable data indicating an extraction of chemically treated tissue frozen in vivo, for example. This can be accomplished by including special-purpose instruction sequences or special-purpose-circuit designs for this function, for example, in optical or other known circuit fabrication operations, in programming by various known voltage modulation techniques, or otherwise as described herein or known by those skilled in the art. Output data 3121, 3122 from such a component in primary system 3100 or network 3190 may be recorded by writing to or otherwise configuring available portions of storage device(s) 3143.

Alternatively or additionally, such specific output data may be transmitted by configuring transistors, relays, or other drivers or conduits 3142 of primary system 3100 to transfer it to component 3113, for example. Component 3112 may perform operation 2520 via implementation as logic for generating at least some of the device-detectable data, for example. Implementation output data 3122 from such a component in primary system 3100 or network 3190 may be recorded into available portions of storage device(s) 3143 or sent to component 3113, for example. Component 3113 may perform operation 2580 via implementation as logic for transmitting an evaluation of the device-detectable data. Output 3102 from flow 2500 may likewise include other data 3123 as described herein. Each portion of implementation 3103 may likewise include one or more instances of software, hardware, or the like implementing logic that may be expressed in several respective forms as described herein or otherwise understood by those skilled in the art.

Referring again to FIG. 26, some instance of flow 2600 may be implemented entirely within primary system 3100. Operation 2640 may be implemented by configuring one or more components 3114, 3115 as logic for obtaining device-detectable data indicating a treatment of a tissue sample in a chamber extended into tissue of an organism, for example, such as by including special-purpose instruction sequences or special-purpose-circuit designs for this function. Output data 3124, 3125 from such a component in primary system 3100 or network 3190 may be recorded into available portions of storage device(s) 3143 or sent to component 3116, for example. Component 3115 may perform operation 2630 via implementation as logic for generating at least some of the device-detectable data, for example. Implementation output data 3125 from such a component in primary system 3100 or network 3190 may be recorded into available portions of storage device(s) 3143 or sent to component 3116, for example. Component 3116 may perform operation 2670 via implementation as logic for transmitting an evaluation of the device-detectable data. Output 3104 from flow 2600 may likewise include other data 3126 as described herein. Each portion of implementation 3103 may likewise include one or more instances of software, hardware, or the like implementing logic that may be expressed in several respective forms as described herein or otherwise understood by those skilled in the art.

Referring again to FIG. 27, some instance of flow 2700 may be implemented entirely within primary system 3100. Operation 2750 may be implemented by configuring one or more components 3117, 3118 as logic for obtaining device-detectable data indicating a treatment of a tissue sample in a chamber extended into tissue of an organism, for example, such as by including special-purpose instruction sequences or special-purpose-circuit designs for this function. Output data 3127 from such a component in primary system 3100 or network 3190 may be recorded into available portions of storage device(s) 3143 or sent to component 3118, for example. Component 3117 may perform operation 2750 via implementation as logic for generating at least some of the device-detectable data, for example. Implementation output data 3127 from such a component in primary system 3100 or network 3190 may be recorded into available portions of storage device(s) 3143 or sent to component 3118, for example. Component 3116 may perform operation 2770 via implementation as logic for transmitting an evaluation of the device-detectable data. Output 3104 from flow 2700 may likewise include other data 3128 as described herein. Each portion of implementation 3103 may likewise include one or more instances of software, hardware, or the like implementing logic that may be expressed in several respective forms as described herein or otherwise understood by those skilled in the art.

In some embodiments, output device 3136 may indicate an occurrence of flow 2500 concisely as a decision, an evaluation, an effect, an hypothesis, a probability, a notification, or some other useful technical result. For example, such “indicating” may comprise such modes as showing, signifying, acknowledging, updating, explaining, associating, or the like in relation to any past or ongoing performance of such actions upon the common item(s) as recited. Such indicating may also provide one or more specifics about the occurrence: the parties or device(s) involved, a description of the method or performance modes used, any sequencing or other temporal aspects involved, indications of resources used, location(s) of the occurrence, implementation version indications or other update-indicative information, or any other such contextual information that may be worthwhile to provide at potential output destinations.

Concise indication may occur, for example, in a context in which at least some items of data 3121-3128 do not matter, or in which a recipient may understand or access portions of data 3121-3128 without receiving a preemptive explanation of how it was obtained. By distilling at least some output 3102, 3104, 3106 at an “upstream” stage (which may comprise integrated circuit 3134, for example, in some arrangements), downstream-stage media (such as other elements of network 3190, for example) may indicate occurrences of various methods described herein more effectively. Variants of flow 2500, for example, may be enhanced by distillations described herein, especially in bandwidth-limited transmissions, security-encoded messages, long-distance transmissions, complex images, or compositions of matter bearing other such expressions.

In some variants, a local implementation comprises a service operable for accessing a remote system running a remote implementation. In some embodiments, such “accessing” may include one or more instances of establishing or permitting an interaction between the server and a local embodiment such that the local embodiment causes or uses another implementation or output of one or more herein-described functions at the server. Functioning as a web browser, remote terminal session, or other remote activation or control device, for example, interface(s) 3130 may interact with one or more primary system users via input and output devices 3136, 3138 so as to manifest an implementation in primary system 3100 via an interaction with server 3194, for example, running a secondary implementation of flow 2500. Such local implementations may comprise a visual display supporting a local internet service to the remote server, for example. Such a remote server may control or otherwise enable one or more instances of hardware or software operating the secondary implementation outside a system, network, or physical proximity of primary system 3100. For a building implementing primary system 3100, for example, “remote” devices may include those in other countries, in orbit, or in adjacent buildings. In some embodiments, “running an implementation” may include invoking one or more instances of software, hardware, firmware, or the like atypically constituted or adapted to facilitate methods or functions as described herein. For example, primary system 3100 running an implementation of flow 2500 may be a remote activation of a special-purpose computer program resident on server 3194 via an internet browser session interaction through linkage 3150, mediated by input device 3138 and output device 3136.

In some variants, some or all of components 3111-3118 may be borne in various data-handling elements—e.g., in one or more instances of storage devices 3143, in memories 3148 or volatile media, passing through linkage 3150 with network 3190 or other conduits 3142, in one or more registers or data-holding devices 3149, or the like. For example, such processing or configuration may occur in response to user data or the like received at input device 3138 or may be presented at output device 3136. Instances of input devices 3138 may (optionally) include one or more instances of cameras or other optical devices, hand-held systems or other portable systems, keypads, sensors, or the like as described herein. Output device(s) 3136 may likewise include one or more instances of image projection modules, touch screens, wrist-wearable systems or the like adapted to be worn while in use, headphones and speakers, eyewear, liquid crystal displays (LCDs), actuators, lasers, organic or other light-emitting diodes, phosphorescent elements, portions of (hybrid) input devices 3138, or the like.

A device-detectable implementation of variants described herein with reference to flows 2500, 2600, 2700, for example, may be divided into several components 3111-3118 carried by one or more instances of active modules such as signal repeaters 3191, communication satellites 3193, servers 3194, processors 3195, routers 3197, or the like. For example, in some embodiments, component 3112 may be borne by an “upstream” module (e.g., repeater 3191 or the like) while or after component 3111 is borne in a “downstream” module (e.g., another instance of repeater 3191, communication satellite 3193, server 3194, or the like). Such downstream modules may “accept” such bits or other portions of implementation 3103 or implementation 3101 sequentially, for example, such as by amplifying, relaying, storing, checking, or otherwise processing what was received actively. Sensors and other “upstream” modules may likewise “accept” raw data, such as by measuring physical phenomena or accessing one or more databases.

An embodiment provides an instrument 1110 having at least (a) a chamber 551, 1748, 2155 or other cavity in which one or more sample treatment protocols 443, 2083 may be applied to a tissue sample 1112, 2062 and (b) sensors, transmitters 3132, invocation logic 2455, or other such output modules configured to transmit one or more measurements 1661, images 1662, records 1690, or other results 192 of such treatment. In some variants, for example, the instrument may include or otherwise interact with a treatment module 890 configured to apply one or more fixatives 3074, optical treatments, marking agents 2165 or other compounds 3075, or other such treatments.

Another embodiment provides a probe 210, 1510, 1610 having one or more separable extraction modules 660, 3000 or other probe portions 2272 (positionable in a digital microscope 2270 or other such equipment, e.g.). The embodiment further provides a buffer-containing or other compound 3075 (in a dispenser having access to a sample 3080, for example) for treating an extraction 1555, 2452 in the module(s), and (c) one or more instances of interface logic 1290, sensors 1644, invocation logic 2455, transmitters 3132, or other output modules configured to transmit one or more measurements 1661, images 1662, records 1690, or other results 192 of such treatment from the probe.

In some embodiments, a medium bearing data (or other such event) may be “caused” (directly or indirectly) by one or more instances of prior or contemporaneous measurements, decisions, transitions, circumstances, or other causal determinants. Any such event may likewise depend upon one or more other prior, contemporaneous, or potential determinants, in various implementations as taught herein. In other words, such events may occur “in response” to both preparatory (earlier) events and triggering (contemporaneous) events in some contexts. Output 3102 may result from more than one component of implementations 3101, 3103 or more than one operation of flow 2500, for example.

In some embodiments, such integrated circuits 3134 may comprise transistors, capacitors, amplifiers, latches, converters, or the like on a common substrate of a semiconductor material, operable to perform computational tasks or other transformations. An integrated circuit may be application-specific (“ASIC”) in that it is designed for a particular use rather than for general purpose use. An integrated circuit may likewise include one or more instances of memory circuits, processors, field-programmable gate arrays (FPGA's), antennas, or other components, and may be referred to as a system-on-a-chip (“SoC”).

In some embodiments, one or more instances of integrated circuits or other processors may be configured to perform auditory pattern recognition. In FIG. 31, for example, instances of the one or more input devices 3138 may include a microphone or the like operable to provide auditory samples in data 3121-3128. Some form or portion of such output may be provided remotely, for example, to one or more instances of neural networks or other configurations of remote processors 3195 operable to perform automatic or supervised speech recognition, selective auditory data retention or transmission, or other auditory pattern recognition, upon the samples. Alternatively or additionally such sound-related data may include annotative information relating thereto such as a capture time or other temporal indications, capture location or other source information, language or other content indications, decibels or other measured quantities, pointers to related data items or other associative indications, or other data aggregations or distillations as described herein.

In some embodiments, one or more instances of integrated circuits or other processors may be configured for optical image pattern recognition. In FIG. 31, for example, instances of lenses 3131 or other input devices 3138 may include optical sensors or the like operable to provide one or more of geometric, hue, or optical intensity information in data 3121-3128. Some form or portion of such output may be provided locally, for example, to one or more instances of optical character recognition software, pattern recognition processing resources, or other configurations of integrated circuits 3134 operable to perform automatic or supervised image recognition, selective optical data retention or transmission, or the like. Alternatively or additionally such image-related data may include annotative information relating thereto such as a capture time or other temporal indications, capture location or other source information, language or other content indications, pointers to related data items or other associative indications, or other data aggregations or distillations as described herein.

In some embodiments, one or more instances of integrated circuits or other processors may be configured to perform linguistic pattern recognition. In FIG. 31, for example, instances of input devices 3138 may include keys, pointing devices, microphones, sensors, reference data, or the like operable to provide spoken, written, or other symbolic expressions in data 3121-3128. Some form or portion of such output may be provided locally, for example, to one or more instances of translation utilities, compilers, or other configurations of integrated circuits 3134 operable to perform automatic or supervised programming or other language recognition, selective linguistic data retention or transmission, or the like. Alternatively or additionally such language-related data may include annotative information relating thereto such as a capture time or other temporal indications, capture location or other source information, language or other content indications, pointers to related data items or other associative indications, or other data classifications, aggregations, or distillations as described herein.

In some embodiments, one or more antennas 3135 or receivers 3133 may include a device that is the receiving end of a communication channel as described herein. For example, such a receiver may gather a signal from a dedicated conduit or from the environment for subsequent processing and/or retransmission. As a further example, such antennas or other receivers may include one or more instances of wireless antennas, radio antennas, satellite antennas, broadband receivers, digital subscriber line (DSL) receivers, modem receivers, transceivers, or configurations of two or more such devices for data reception as described herein or otherwise known.

In one variant, two or more respective portions of output data 3121-3128 may be sent from server 3194 through respective channels at various times, one portion passing through repeater 3191 and another through router 3197. Such channels may each bear a respective portion of a data aggregation or extraction, a publication, a comparative analysis or decision, a record selection, digital subscriber content, statistics or other research information, a resource status or potential allocation, an evaluation, an opportunity indication, a test or computational result, or some other output 3102, 3104, 3106 of possible interest. Such distributed media may be implemented as an expedient or efficient mode of bearing such portions of output data to a common destination such as interface 3130 or holding device 3149. Alternatively or additionally, some such data may be transported by moving a medium (carried on storage device 3143, for example) so that only a small portion (a purchase or other access authorization, for example, or a contingent or supplemental module) is transferred via linkage 3150.

In some embodiments, one or more instances of signal repeaters 3191 may include a device or functional implementation that receives a signal and transmits some or all of the signal with one or more of an altered strength or frequency, or with other modulation (e.g., an optical-electrical-optical amplification device, a radio signal amplifier or format converter, a wireless signal amplifier, or the like). A repeater may convert analog to digital signals or digital to analog signals, for example, or perform no conversion. Alternatively or additionally, a repeater may reshape, retime or otherwise reorder an output for transmission. A repeater may likewise introduce a frequency offset to an output signal such that the received and transmitted frequencies are different. A repeater also may include one or more instances of a relay, a translator, a transponder, a transceiver, an active hub, a booster, a noise-attenuating filter, or the like.

In some embodiments, such communication satellite(s) 3193 may be configured to facilitate telecommunications while in a geosynchronous orbit, a Molniya orbit, a low earth orbit, or the like. Alternatively or additionally, a communication satellite may receive or transmit, for example, telephony signals, television signals, radio signals, broadband telecommunications signals, or the like.

In some variants, processor 3195 or any components 3111-3118 of implementations 3103, 3101 may (optionally) be configured to perform flow variants as described herein with reference to FIGS. 25-27. An occurrence of such a variant can be expressed as a computation, a transition, or as one or more other items of data 3121-3128 described herein. Such output 3104,2802 can be generated, for example, by depicted components of primary system 3100 or network 3190 including one or more features as described with reference to FIGS. 1-24.

Some variants may include special-purpose circuitry for implementing a spectroscopic analysis protocol. In light of teachings herein, numerous existing techniques may be applied for implementing such modules 2855 of evaluation logic 2850 as described herein without undue experimentation. See, e.g., U.S. Pat. No. 7,411,396 (“Method and system of magnetic resonance spectroscopy with volume element dissection”); U.S. Pat. No. 7,356,364 (“Device for optical monitoring of constituent in tissue or body fluid sample using wavelength modulation spectroscopy, such as for blood glucose levels”); U.S. Pat. No. 7,149,567 (“Near-infrared spectroscopic tissue imaging for medical applications”); U.S. Pat. No. 6,697,665 (“Systems and methods of molecular spectroscopy to provide for the diagnosis of tissue”); U.S. Pat. No. 6,697,652 (“Fluorescence, reflectance and light scattering spectroscopy for measuring tissue”); U.S. Pat. No. 6,690,966 (“Methods of molecular spectroscopy to provide for the diagnosis of tissue”); U.S. Pat. No. 6,681,133 (“Methods and apparatus for obtaining enhanced spectroscopic information from living tissue”); U.S. Pat. No. 6,671,540 (“Methods and systems for detecting abnormal tissue using spectroscopic techniques”); U.S. Pat. No. 6,642,059 (“Method for the comparative quantitative analysis of proteins and other biological material by isotopic labeling and mass spectroscopy”); U.S. Pat. No. 6,324,418 (“Portable tissue spectroscopy apparatus and method”); U.S. Pat. No. 6,289,230 (“Tissue modulation process for quantitative noninvasive in vivo spectroscopic analysis of tissues”); U.S. Pat. No. 6,157,856 (“Tissue diagnostics using evanescent spectroscopy”); U.S. Pat. No. 6,095,982 (“Spectroscopic method and apparatus for optically detecting abnormal mammalian epithelial tissue”). Alternatively or additionally, such modules may comprise or otherwise interact with conduits 3142 or other media 100, 1000 bearing an operational setting value 2453 usable in mass spectroscopes 2065, imaging systems, or other such equipment for analyzing solid or other samples 1112, 2062, 3080 as described herein.

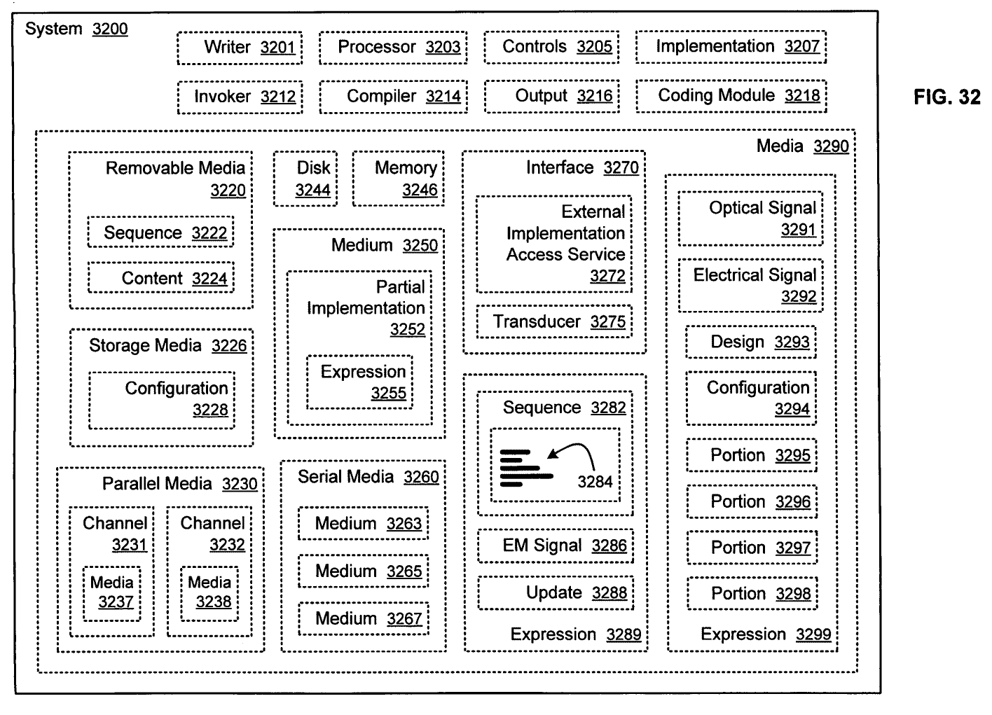

With reference now to FIG. 32, shown is an example of another system that may serve as a context for introducing one or more processes, systems or other articles described herein. As shown system 3200 comprises one or more instances of writers 3201, processors 3203, controls 3205, software or other implementations 3207, invokers 3212, compilers 3214, outputs 3216, coding modules 3218, or the like with one or more media 3290 bearing expressions or outputs thereof. In some embodiments, such media may include distributed media bearing a divided or otherwise distributed implementation or output. For example, in some embodiments, such media may include two or more physically distinct solid-state memories, two or more transmission media, a combination of such transmission media with one or more data-holding media configured as a data source or destination, or the like.

In some embodiments, transmission media may be “configured” to bear an output or implementation (a) by causing a channel in a medium to convey a portion thereof or (b) by constituting, adapting, addressing, or otherwise linking to such media in some other mode that depends upon one or more atypical traits of the partial or whole output or implementation. Data-holding elements of media may likewise be “configured” to bear an output or implementation portion (a) by holding the portion in a storage or memory location or (b) by constituting, adapting, addressing, or otherwise linking to such media in some other mode that depends upon one or more atypical traits of the partial or whole output or implementation. Such atypical traits may include a name, address, portion identifier 2911, functional description, or the like sufficient to distinguish the output, implementation, or portion from a generic object.

In some embodiments described herein, “logic” and similar implementations can include software or other control structures operable to guide device operation. Electronic circuitry, for example, can manifest one or more paths of electrical current constructed and arranged to implement various logic functions as described herein. In some embodiments, one or more media are “configured to bear” a device-detectable implementation if such media hold or transmit a special-purpose device instruction set operable to perform a novel method as described herein. Alternatively or additionally, in some variants, an implementation may include special-purpose hardware or firmware components or general-purpose components executing or otherwise invoking special-purpose components. Specifications or other implementations may be transmitted by one or more instances of transmission media as described herein, optionally by packet transmission or otherwise by passing through distributed media at various times.

In some embodiments, one or more of the coding modules 3218 may be configured with circuitry for applying, imposing, or otherwise using a syntactic or other encoding constraint in forming, extracting, or otherwise handling respective portions of the device-detectable implementation or output. In encoding a software module or other message content, for example, compiler 3214 or coding module 3218 may implement one or more such constraints pursuant to public key or other encryption, applying error correction modes, certifying or otherwise annotating the message content, or implementing other security practices described herein or known by those skilled in the art. Alternatively or additionally, another instance of coding module 3218 may be configured to receive data (via receiver 3133, e.g.) and decode or otherwise distill the received data using one or more such encoding constraints. Compiler 3214 may, in some variants, convert one or more of components 3111-3118 from a corresponding source code form before the component(s) are transmitted across linkage 3150.

System 3200 may be implemented, for example, as one or more instances of stand-alone workstations, servers, vehicles, portable devices, removable media 3220, as components of primary system 3100 or network 3190 (of FIG. 31), or the like. Alternatively or additionally, media 3290 may include one or more instances of signal repeaters 3191, communication satellites 3193, servers 3194, processors 3195, routers 3197, portions of primary system 3100 as shown, or the like.

Media 3290 may include one or more instances of removable media 3220, tapes or other storage media 3226; parallel (transmission) media 3230; disks 3244; memories 3246; other data-handling media 3250; serial media 3260; interfaces 3270; or expressions 3289, 3299. Removable media 3220 can bear one or more device-detectable instances of instruction sequences 3222 or other implementations of flow 2500 or flow 2600, for example. Alternatively or additionally, in some embodiments, removable media 3220 can bear alphanumeric data, audio data, image data, structure-descriptive values, or other content 3224 in a context that indicates an occurrence of one or more flows 2500, 2600, 2700. In some circumstances, transmission media may bear respective portions of implementations as described herein serially or otherwise non-simultaneously. In some variants in which two portions 3297, 3298 constitute a partial or complete software implementation or product of a novel method described herein, portion 3297 may follow portion 3298 successively through serial media 3263, 3265, 3267 (with transmission of portion 3297 partly overlapping in time with transmission of portion 3298 passing through medium 3263, for example). As shown, parallel channels 3231, 3232 are respectively implemented at least in media 3237, 3238 of a bus or otherwise effectively in isolation from one another. In some embodiments, a bus may be a system of two or more signal paths—not unified by a nominally ideal conduction path between them—configured to transfer data between or among internal or external computer components. For example, one data channel may include a power line (e.g., as medium 3265) operable for transmitting content of the device-detectable implementation as described herein between two taps or other terminals (e.g., as media 3263, 3267 comprising a source and destination). In another such configuration, one or more media 3237 of channel 3231 may bear portion 3297 before, while or after one or more other media 3238 of parallel channel 3232 bear portion 3298. In some embodiments, such a process may occur “while” another process occurs if they coincide or otherwise overlap in time substantially (by several clock cycles, for example). In some embodiments, such a process may occur “after” an event if any instance of the process begins after any instance of the event concludes, irrespective of other instances overlapping or the like.

In a variant in which a channel through medium 3250 bears an expression 3255 partially implementing an operational flow described herein, the remainder of the implementation may be borne (earlier or later, in some instances) by the same medium 3250 or by one or more other portions of media 3290 as shown. In some embodiments, moreover, one or more controls 3205 may configure at least some media 3290 by triggering transmissions as described above or transmissions of one or more outputs 3216 thereof.

In some embodiments, the one or more “physical media” may include one or more instances of conduits, layers, networks, static storage compositions, or other homogenous or polymorphic structures or compositions suitable for bearing signals. In some embodiments, such a “communication channel” in physical media may include a signal path between two transceivers or the like. A “remainder” of the media may include other signal paths intersecting the communication channel or other media as described herein. In some variants, another exemplary system comprises one or more physical media 3290 constructed and arranged to receive a special-purpose sequence 3282 of two or more device-detectable instructions 3284 for implementing a flow as described herein or to receive an output of executing such instructions. Physical media 3290 may (optionally) be configured by writer 3201, transmitter 3132, or the like.

In some embodiments, such a “special-purpose” instruction sequence may include any ordered set of two or more instructions directly or indirectly operable for causing multi-purpose hardware or software to perform one or more methods or functions described herein: source code, macro code, controller or other machine code, or the like. In some embodiments, an implementation may include one or more instances of special-purpose sequences 3282 of instructions 3284, patches or other implementation updates 3288, configurations 3294, special-purpose circuit designs 3293, or the like. Such “designs,” for example, may include one or more instances of a mask set definition, a connectivity layout of one or more gates or other logic elements, an application-specific integrated circuit (ASIC), a multivariate transfer function, or the like.

Segments of such implementations or their outputs may (optionally) be manifested one or more information-bearing static attributes comprising the device-detectable implementation. Such attributes may, in some embodiments, comprise a concentration or other layout attribute of magnetic or charge-bearing elements, visible or other optical elements, or other particles in or on a liquid crystal display or other solid-containing medium. Solid state data storage modules or other such static media may further comprise one or more instances of laser markings, barcodes, human-readable identifiers, or the like, such as to indicate one or more attributes of the device-detectable implementation. Alternatively or additionally such solid state or other solid-containing media may include one or more instances of semiconductor devices or other circuitry, magnetic or optical digital storage disks, dynamic or flash random access memories (RAMs), or the like. Magnetoresistive RAMs may bear larger implementation or output portions or aggregations safely and efficiently, moreover, and without any need for motors or the like for positioning the storage medium.

Segments of such implementations or their outputs may likewise be manifested in electromagnetic signals 3286, laser or other optical signals 3291, electrical signals 3292, or the like. In some embodiments, for example, such electrical or electromagnetic signals may include one or more instances of static or variable voltage levels or other analog values, radio frequency transmissions or the like. In some embodiments, the above-mentioned “optical” signals may likewise include one or more instances of time- or position-dependent, device-detectable variations in hue, intensity, or the like. Alternatively or additionally, portions of such implementations or their outputs may manifest as one or more instances of magnetic, magneto-optic, electrostatic, or other physical configurations 3228 of nonvolatile storage media 3226 or as external implementation access services 3272.

In some embodiments, physical media can be configured by being “operated to bear” or “operated upon to bear” a signal. For example, they may include physical media that generate, transmit, conduct, receive, or otherwise convey or store a device-detectable implementation or output as described herein. Such conveyance or storing of a device-detectable implementation or output may be carried out in a distributed fashion at various times or locations, or such conveyance or storing of a device-detectable implementation or output may be done at one location or time. As discussed above, such physical media “operated to bear” or “operated upon to bear” may include physical media that are atypically constituted or adapted to facilitate methods or functions as described herein.

In some configurations, one or more output devices 3136 may present one or more results of generating at least some of the device-detectable data in response to interface(s) 3130 receiving one or more invocations or outputs of an implementation of this function via linkage 3150. Such an “invocation” may, in some embodiments, comprise one or more instances of requests, hardware or software activations, user actions, or other determinants as described herein. Alternatively or additionally, in some embodiments, one or more input devices 3138 may later receive one or more invocations or results of transmitting an evaluation of the device-detectable data. In contexts like these, processor 3195 or other components of network 3190 may likewise constitute a secondary implementation having access to a primary instance of interface 3130 implementing methods like flow 2500 as described herein.

Serial media 3260 comprises a communication channel of two or more media configured to bear a transition or other output increment successively. In some embodiments, for example, serial media 3260 may include a communication line or wireless medium (e.g., as medium 3265) between two signal-bearing conduits (e.g., terminals or antennas as media 3263, 3267). Alternatively or additionally, one or more lenses 3131 or other light-transmissive media may comprise a serial medium between a light-transmissive medium and a sensor or other light receiver 3133 or transmitter 3132. In some embodiments, such “light-transmissive” media may (optionally) comprise metamaterials or other media operable for bearing one or more instances of microwave signals, radiowave signals, visible light signals, or the like.

In some embodiments, such a lens may be an optical element that causes light to converge or diverge along one or more signal paths. Such a light-transmissive medium may include a signal-bearing conduit, glass, or other physical medium through which an optical signal may travel. More generally, a signal-bearing conduit may be an electrical wire, a telecommunications cable, a fiber-optic cable, or a mechanical coupling or other path for the conveyance of analog or digital signals.

An embodiment provides a probe 210, 370 or other device comprising (a) a handling control surface 214, 1630, (b) one or more distal portions 1740 narrow enough to extend into a living organism 1210, (c) a first dispenser 921, 922, 1540 configured to apply an agent, compound 3075, or other treatment material(s) to tissue 985, 1531 adjacent the device, and (d) one or more instances of interface logic 1270, transducers 1290, transmitters 3132, invocation logic 2455, or other modules of output 3216 configured to transmit a result 1194, 1663 of the treatment material(s).

An embodiment provides one or more physical media 3290 bearing (a) an earlier image depicting at least some of a cell to which an optical enhancement material was applied in vivo and (b) a later image depicting at least some of the cell to which the optical enhancement material was applied in vivo. This can occur, for example, in a context in which the material comprises a vital stain and in which the two or more images illustrate a progressive change in the cell in its environment.

An embodiment provides one or more disks 3244 or other physical media 3290 bearing an optical signal 3291 or other go/no-go indicator 2931 expressing an evaluation of tissue 1531 to which a fluorescent or other optical enhancement material was applied in vivo, for example, via one or more protocols 1571, 1731, 2081 as described herein. Alternatively or additionally, such media may bear device-detectable data indicating a treatment of a tissue component in a chamber 1515, 1748 extended into tissue 985, 1531 of an organism 1210. Alternatively or additionally, such media may bear a laser-scanned image of (at least) some of a cell to which an elutant 1363, fluor, or other marking component was included in a compound 3075 applied in vivo.

Alternatively or additionally, system 3200 may include one or more instances of media for handling implementations or their outputs: satellite dishes or other reflectors 3137, antennas 3135 or other transducers 3275, arrays of two or more such devices configured to detect or redirect one or more incoming signals, caching elements or other data-holding elements (e.g., disks 3244, memories 3246, or other media 3290), integrated circuits 3134, or the like. In some variants, one or more media may be “configured” to bear a device-detectable implementation as described herein by being constituted or otherwise specially adapted for that type of implementation at one or more respective times, overlapping or otherwise. Such “signal-bearing” media may include those configured to bear one or more such signals at various times as well as those currently bearing them.

In some variants, such caching elements may comprise a circuit or device configured to store data that duplicates original values stored elsewhere or computed earlier in time. For example, a caching element may be a temporary storage area where frequently-accessed data may be held for rapid access by a computing system. A caching element likewise may be machine-readable memory (including computer-readable media such as random access memory or data disks). In some embodiments, such caching elements may likewise comprise a latching circuit or device configured to store data that has been modified from original values associated with the data (held elsewhere or computed earlier in time, for example).

In one variant, respective portions 3295, 3296 of an expression 3299 of implementation 3207 may be sent through respective channels at various times. Invoker 3212 may request or otherwise attempt to activate a computer program or streaming media overseas via a telephone cable or other channel 3231. Meanwhile, output 3216 may attempt to trigger a session or other partial implementation 3252, success in which may be indicated by receiving expression 3255 into a visual display or other medium 3250. Such a program or other implementation may be made complete, for example, once both of these attempts succeed.

In some embodiments, transducer(s) 3275 may comprise one or more devices that convert a signal from one form to another form. For example, a transducer may be a cathode ray tube that transforms electrical signals into visual signals. Another example of a transducer comprises a microelectromechanical systems (“MEMS”) device, which may be configured to convert mechanical signals into electrical signals (or vice versa).

Some variants may include special-purpose circuitry for triggering a diagnostic or other evaluation in one or more microfluidic structures. In light of teachings herein, numerous existing techniques may be applied for implementing such control modules 2963 as described herein without undue experimentation. See, e.g., U.S. Pat. No. 7,411,672 (“Method and apparatus for chemical imaging in a microfluidic circuit”); U.S. Pat. No. 7,391,936 (“Microfluidic sensors and methods for making the same”); U.S. Pat. No. 7,336,812 (“System for microvolume laser scanning cytometry”); U.S. Pat. No. 7,315,357 (“Imaging and analyzing parameters of small moving objects such as cells”); U.S. Pat. No. 7,312,611 (“Apparatus and method for trapping bead based reagents within microfluidic analysis systems”); U.S. Pat. No. 7,264,794 (“Methods of in vivo cytometry”); U.S. Pat. No. 7,214,478 (“Composite material for biological or biochemical analysis microfluidic system”); U.S. Pat. No. 7,186,352 (“Microfluidic systems with embedded materials and structures and method thereof”); U.S. Pat. No. 7,160,730 (“Method and apparatus for cell sorting”); U.S. Pat. No. 7,125,711 (“Method and apparatus for splitting of specimens into multiple channels of a microfluidic device”); U.S. Pat. No. 7,081,192 (“Methods for manipulating moieties in microfluidic systems”). Some such variants, for example, may include parallel or other media 3290 bearing a signal from one or more chemical sensors as described herein. Alternatively or additionally, such modules may comprise or otherwise interact with media bearing one or more resource addresses, invocation parameters, or other such values 2956, 2958 usable in a module 2454 of invocation logic 2455 as described herein.

Alternatively or additionally, some variants may include or otherwise interact with one or more modules and/or protocols for controlling an ablation, extraction, or other operational element adjacent to tissue or other extractions. In light of teachings herein, numerous existing techniques may be applied for configuring one or more modules 2846 of control logic 2840 to implement such features as described herein without undue experimentation. See, e.g., U.S. Pat. No. 7,384,417 (“Air-powered tissue-aspiration instrument system employing curved bipolar-type electro-cauterizing dual cannula assembly”); U.S. Pat. No. 7,332,160 (“Medical device and method for tissue removal and repair”); U.S. Pat. No. 7,297,145 (“Bipolar electrosurgical clamp for removing and modifying tissue”); U.S. Pat. No. 7,270,661 (“Electrosurgical apparatus and methods for treatment and removal of tissue”); U.S. Pat. No. 7,186,234 (“Electrosurgical apparatus and methods for treatment and removal of tissue”); U.S. Pat. No. 6,918,919 (“System and method for bracketing and removing tissue”); U.S. Pat. No. 6,852,108 (“Apparatus and method for resecting and removing selected body tissue from a site inside a patient”); U.S. Pat. No. 6,830,556 (“Debridement extension providing irrigation and mechanical scrubbing for removal of dead, devitalized, or contaminated tissue from a wound”); U.S. Pat. No. 6,761,718 (“Direction-oriented and spatially controlled bipolar coagulator for in-situ cauterization of adherent cranial tissue occluding a ventricular catheter previously implanted in-vivo”); U.S. Pat. No. 6,652,522 (“Power-assisted tissue aspiration instrument with cauterizing cannula assembly”); U.S. Pat. No. 6,401,722 (“Method for stabilizing and removing tissue”); U.S. Pat. No. 6,296,608 (“Diagnosing and performing interventional procedures on tissue in vivo”). Some such variants, for example, may include or otherwise interact with a memory 3246 or other media 100, 3290 bearing one or more values 2453, 2953 as described herein. Such values may, in various contexts, be usable in configuring operational settings of a probe 210, 370, 590, 840, 910, 1510; a digital microscope 2270; a flow cytometer 2414, an extraction module 3000, or other such equipment for preparing, imaging, or otherwise handling samples 1112, 2062, 3080. Such variants may, for example, include media bearing one or more frequency ranges, acquisition durations, or other such values 2953, 2955 usable in an interferometer 2404 as described herein.

Alternatively or additionally, some variants may include hardware configurations for a surgical probe or other instrument with a handling control surface 1630. In light of teachings herein, numerous existing techniques may be applied for implementing and positioning such extraction modules 660, 850, 3000; sensors 553, 1644, 1746 or other detection logic; treatment modules 530, 890; distal portions 550, 1740; control logic 2840, transmission or other media 3290, chambers or other features for tissue sampling and/or observation, or other features as described herein without undue experimentation. See, e.g., U.S. Pat. No. 7,366,562 (“Method and apparatus for surgical navigation”); U.S. Pat. No. 7,328,057 (“Shunt passer or like surgical instrument configured for receiving different-sized positioning locators of image-guided surgical system”); U.S. Pat. No. 7,252,660 (“Multifunctional instrument for use in microinvasive surgery”); U.S. Pat. No. 7,166,114 (“Method and system for calibrating a surgical tool and adapter thereof”); U.S. Pat. No. 7,122,028 (“Reconfiguration surgical apparatus”); U.S. Pat. No. 6,950,691 (“Surgery support system and surgery support method”); U.S. Pat. No. 6,802,840 (“Medical instrument positioning tool and method”); U.S. Pat. No. 6,647,281 (“Expandable diagnostic or therapeutic apparatus and system for introducing the same into the body”); U.S. Pat. No. 6,589,231 (“Multi-function surgical instrument tool actuator assembly”); U.S. Pat. No. 6,572,264 (“Radiation clinical thermometer”); U.S. Pat. No. 6,497,134 (“Calibration of an instrument”); U.S. Pat. No. 6,428,547 (“Detection of the shape of treatment devices”); U.S. Pat. No. 6,298,262 (“Instrument guidance for stereotactic surgery”); U.S. Pat. No. 6,197,003 (“Catheter advancing single-handed soft passer”). Some such variants, for example, may present or otherwise bear laser-scanned images, measurements, evaluations, or other such output relating to a patient's tissue. Alternatively or additionally, such output may include products of various botanical or other agricultural protocols 2082, minimally invasive protocols 1733, or other surgical protocols as described herein.

Alternatively or additionally, some variants may include special-purpose protocols or components for causing cells or other structures to undergo scanning or other electron microscopic imaging. In light of teachings herein, numerous existing techniques may be applied by module 2961 for implementing such sampling, marking, or other protocols without undue experimentation. See, e.g., U.S. Pat. No. 7,374,907 (“System and method for automatically processing tissue samples”); U.S. Pat. No. 7,344,700 (“Radiolabeled selective androgen receptor modulators and their use in prostate cancer imaging and therapy”); U.S. Pat. No. 7,230,242 (“Methods for SEM inspection of fluid containing samples”); U.S. Pat. No. 6,811,766 (“Ultrasound imaging with contrast agent targeted to microvasculature and a vasodilator drug”); U.S. Pat. No. 6,783,752 (“Contrast agents”); U.S. Pat. No. 6,106,804 (“Arsenic-72 labeled compounds for tissue specific medical imaging”); U.S. Pat. No. 6,096,874 (“High affinity tamoxifen derivatives”); U.S. Pat. No. 5,808,300 (“Method and apparatus for imaging biological samples with MALDI MS”). This can occur, for example, in a context in which linkage module 2900 interacts with one or more facilities 990, providers 2475 or other resources (in networks 790, 1830, e.g.), or other entities via one or more media 100, 1000, 3290 as described herein.

In some variants, the above-described systems and methods may incorporate or otherwise operate in conjunction with a hand-held probe or other instrument with a handling control surface. Such surfaces may be configured to permit a clinician or other user to extend an entirety of a chamber into an organism, for example, or otherwise to facilitate tissue extractions and/or measurements. Alternatively or additionally, such embodiments may include a context in which a chamber contains a reagent to begin the treatment upon a portion of the tissue entering the chamber.

In some variants, the above-described systems and methods may incorporate or otherwise operate in conjunction with circuitry for transmitting energy into extractions in a chamber, such as for curing a fixative and/or to facilitate capturing an image of a sample.

In some variants, the above-described systems and methods may incorporate or otherwise operate in conjunction with a camera or other imaging system, an electrospray or other mass spectrometer, or other such instrument configured to observe such a tissue sample in the chamber and to transmit, store, display, or otherwise provide at least some such device-detectable data on the one or more physical media.

In some variants, the above-described systems and methods may incorporate or otherwise operate in conjunction with dispensers of pharmaceuticals, fixatives, solvents, or other chemical treatment materials. Such materials may include stains or other optical enhancement materials, for example. Such materials may likewise include a syringe or other such mechanism for depositing materials in vivo and/or into a chamber. Alternatively or additionally, such embodiments may include a context in which the treatment commences upon a portion of the tissue in such a chamber and continues upon the tissue sample in the chamber.

In some variants, the above-described systems and methods may incorporate or otherwise operate in conjunction with optical or other treatment components causing a discoloration, luminescence, or other artificial enhancement of one or more optical properties of a tissue or extraction. In many existing protocols, for example, markers may effectively be used for detecting specific genes or other components of large molecules.

In some variants, the above-described systems and methods may incorporate or otherwise operate in conjunction with physical media bearing one or more configuration parameters, type identifiers, images, measurements, specifications, or other descriptors of instruments and/or materials.

In some variants, the above-described systems and methods may incorporate or otherwise operate in conjunction with various spectrometers, microscopes, ultrasound or magnetic resonance imaging systems, or other such instruments as exemplified herein. Such instruments may, for example, be configured (a) to observe tissue samples in chambers and (b) to include physical media bearing images or other device-detectable data. Such instruments may likewise include one or more lenses configured to receive optical energy from a region containing one or more cells, for example, and circuitry for transforming such optical energy into images.

Some or all of the embodiments described herein may generally comprise technologies for handling one or more bioactive agents and/or carriers in releasable module form, via a liquid-bearing conduit, in a mist or other spray form, in a pumped or other pressurized form, or otherwise according to technologies described herein. In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

All of the above-mentioned U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Those skilled in the art will recognize that it is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, helicopter, etc.) , (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory. Further, implementation of at least part of a system for performing a method in one territory does not preclude use of the system in another territory.

Various aspects of the subject matter described herein are set out in the following numbered clauses:

1. A medical or veterinary system comprising:

a surgical probe having a first separable extraction module;

a treatment module configured to apply a first treatment to a tissue sample in the first separable extraction module of the surgical probe; and

an output module configured to transmit a result of the first treatment from the surgical probe.

2. The medical or veterinary system of clause 1 in which the treatment module comprises:

circuitry for causing an application of the first treatment in response to contemporaneous user input.

3. The medical or veterinary system of clause 1, further comprising:

circuitry for positioning at least the first separable extraction module.

4. The medical or veterinary system of clause 1, further comprising:

circuitry for positioning at least a distal end of the surgical probe.

5. The medical or veterinary system of clause 1, further comprising:

circuitry for processing data from one or more assay protocols performed upon the tissue sample.

6. The medical or veterinary system of clause 1, further comprising:

circuitry for processing data from one or more biomarker detection protocols performed upon the tissue sample.

7. The medical or veterinary system of clause 1, further comprising:

at least one medium bearing an operational setting value usable in laser scanning equipment for analyzing the tissue sample.

8. The medical or veterinary system of clause 1, further comprising:

at least one medium bearing an operational setting value usable in a mass spectroscope for analyzing the tissue sample.

9. The medical or veterinary system of clause 1, further comprising:

at least one medium bearing an operational setting value usable in a microscope for analyzing the tissue sample.

10. The medical or veterinary system of clause 1, further comprising:

laser scanning equipment operable for receiving the first separable extraction module.

11. The medical or veterinary system of clause 1, further comprising:

imaging equipment operable for receiving the first separable extraction module.

12. The medical or veterinary system of clause 1 in which the output module comprises:

one or more conduits operably coupling the first separable extraction module to imaging equipment operable to detect the result of the first treatment.

13. The medical or veterinary system of clause 1, further comprising:

circuitry for configuring the result to include one or more quantifications derived from an optical field of the surgical probe.

14. The medical or veterinary system of clause 1 in which the first treatment comprises:

a drug.

15. The medical or veterinary system of clause 1 in which the first treatment comprises:

a buffer.

16. The medical or veterinary system of clause 1 in which the first treatment comprises:

a permeabilizing agent.

17. The medical or veterinary system of clause 1 in which the first treatment comprises:

one or more of a microinjection or an electropermeabilization.

18. The medical or veterinary system of clause 1, further comprising:

an ultramicrotome configured to section the tissue sample.

19. The medical or veterinary system of clause 1, further comprising:

a device configured to extract the tissue sample by severing a larger tissue sample.

20. The medical or veterinary system of clause 1, in which the output module comprises:

a device configured to observe the tissue sample in a first chamber of the first separable extraction module and to transmit the result via one or more conduits.

21. The medical or veterinary system of clause 1, in which the output module comprises:

circuitry for causing at least some of the result to be stored.

22. The medical or veterinary system of clause 1, further comprising:

a device configured to monitor the tissue sample in the first separable extraction module and to cause a presentation of at least some of the result on one or more physical media.

23. The medical or veterinary system of clause 1, further comprising:

an electron microscope configured to observe the tissue sample and to provide at least some of the result on one or more physical media.

24. The medical or veterinary system of clause 1, further comprising:

a fluorescence microscope configured to observe the tissue sample and to provide at least some of the result on one or more physical media.

25. The medical or veterinary system of clause 1, further comprising:

a confocal microscope configured to observe the tissue sample and to provide at least some of the result on one or more physical media.

26. The medical or veterinary system of clause 1, further comprising:

a spectrometer configured to observe the tissue sample and to provide at least some of the result on one or more physical media.

27. The medical or veterinary system of clause 1, further comprising:

an imaging system configured to observe the tissue sample and to provide at least some of the result on one or more physical media.

28. The medical or veterinary system of clause 1, further comprising:

a nuclear magnetic resonance imaging system configured to observe the tissue sample and to transmit at least some of the result on one or more physical media.

29. The medical or veterinary system of clause 1, further comprising:

circuitry for transmitting energy into the tissue sample; and

circuitry for capturing an image of the tissue sample.

30. The medical or veterinary system of clause 1 in which the surgical probe comprises:

one or more handling control surfaces configured to permit a user to extend an entirety of the first separable extraction module into an organism.

31. The medical or veterinary system of clause 1 in which the surgical probe comprises:

the first separable extraction module containing a chamber and supportable by and separable from a remainder of the surgical probe.

32. The medical or veterinary system of clause 1 in which the surgical probe comprises:

a second extraction module.

33. The medical or veterinary system of clause 1, further comprising:

one or more physical media bearing a descriptor of a device that includes at least the first separable extraction module.

34. The medical or veterinary system of clause 1, further comprising:

one or more media bearing an attribute of a macromolecule of the tissue sample.

35. The medical or veterinary system of clause 1, further comprising:

one or more media bearing a shape-indicative category relating to a portion of the tissue sample.

36. The medical or veterinary system of clause 1, further comprising:

circuitry for selecting a stain effective for indicating whether the tissue sample includes a chromosomal abnormality.

37. The medical or veterinary system of clause 1, further comprising:

circuitry for causing the tissue sample to come into contact with a stain effective for indicating whether the tissue sample apparently exhibits an attribute of interest.

38. The medical or veterinary system of clause 1, further comprising:

one or more media bearing an indication of whether the tissue sample apparently exhibits a chromosomal attribute of interest.

39. The medical or veterinary system of clause 1, in which the first dispenser comprises:

one or more media bearing an indication of how much of the tissue sample apparently exhibits a pathology.

40. The medical or veterinary system of clause 1, in which the first dispenser comprises:

one or more media bearing a go/no-go result.

41. The medical or veterinary system of clause 1, further comprising:

a dispenser operable to mark a portion of the tissue sample with a luminescent material.

42. The medical or veterinary system of clause 1, further comprising:

a dispenser operable to mark a portion of the tissue sample with a stain.

43. The medical or veterinary system of clause 1, further comprising:

circuitry for configuring the result to include one or more size-descriptive quantities relating to the tissue sample.

44. A medical or veterinary system comprising:

a surgical instrument having at least a first cavity and a first treatment module configured to apply a first treatment to a tissue sample in the first cavity; and

an output module configured to transmit a result of the first treatment.

45. The medical or veterinary system of clause 44 in which the first treatment module comprises:

circuitry for causing an application of the first treatment in response to contemporaneous user input.

46. The medical or veterinary system of clause 44, further comprising:

circuitry for positioning at least a distal end of the surgical instrument.

47. The medical or veterinary system of clause 44, further comprising:

circuitry for processing data from one or more assay protocols performed upon the tissue sample.

48. The medical or veterinary system of clause 44, further comprising:

circuitry for processing data from one or more biomarker detection protocols performed upon the tissue sample.

49. The medical or veterinary system of clause 44, further comprising:

at least one medium bearing an operational setting value usable in laser scanning equipment for analyzing the tissue sample.

50. The medical or veterinary system of clause 44, further comprising:

at least one medium bearing an operational setting value usable in a mass spectroscope for analyzing the tissue sample.

51. The medical or veterinary system of clause 44, further comprising:

at least one medium bearing an operational setting value usable in a microscope for analyzing the tissue sample.

52. The medical or veterinary system of clause 44, further comprising:

laser scanning equipment operable for receiving at least some of the surgical instrument.

53. The medical or veterinary system of clause 44, further comprising:

imaging equipment operable for receiving a separable extraction module of the surgical instrument that contains the first cavity.

54. The medical or veterinary system of clause 44 in which the output module comprises:

one or more conduits operably coupling the first cavity to imaging equipment operable to detect the result of the first treatment.

55. The medical or veterinary system of clause 44 in which the first treatment comprises:

a drug.

56. The medical or veterinary system of clause 44 in which the first treatment comprises:

a buffer.

57. The medical or veterinary system of clause 44 in which the first treatment comprises:

a permeabilizing agent.

58. The medical or veterinary system of clause 44 in which the first treatment comprises:

one or more of a microinjection or an electropermeabilization.

59. The medical or veterinary system of clause 44, further comprising:

circuitry for configuring the result to include one or more quantifications derived from an optical field of the tissue sample.

60. The medical or veterinary system of clause 44, further comprising:

a microtome configured to section the tissue sample.

61. The medical or veterinary system of clause 44, further comprising:

a device configured to extract the tissue sample by dividing a larger tissue sample.

62. The medical or veterinary system of clause 44, in which the output module comprises:

a device configured to observe the tissue sample in the first cavity and to transmit the result via one or more conduits.

63. The medical or veterinary system of clause 44, in which the output module comprises:

circuitry for causing the result of the first treatment to be stored on one or more physical media.

64. The medical or veterinary system of clause 44, further comprising:

a device configured to observe the tissue sample in a first separable extraction module and to cause a presentation of at least some of the result on one or more physical media.

65. The medical or veterinary system of clause 44, further comprising:

an electron microscope configured to observe the tissue sample and to provide at least some of the result on one or more physical media.

66. The medical or veterinary system of clause 44, further comprising:

a fluorescence microscope configured to observe the tissue sample and to provide at least some of the result on one or more physical media.

67. The medical or veterinary system of clause 44, further comprising:

a confocal microscope configured to observe the tissue sample and to provide at least some of the result on one or more physical media.

68. The medical or veterinary system of clause 44, further comprising:

a spectrometer configured to observe the tissue sample and to provide at least some of the result on one or more physical media.

69. The medical or veterinary system of clause 44, further comprising:

an imaging system configured to observe the tissue sample and to provide at least some of the result on one or more physical media.

70. The medical or veterinary system of clause 44, further comprising:

a nuclear magnetic resonance imaging system configured to observe the tissue sample and to provide at least some of the result on one or more physical media.

71. The medical or veterinary system of clause 44, further comprising:

circuitry for transmitting energy into the tissue sample; and

circuitry for capturing an image of the tissue sample.

72. The medical or veterinary system of clause 44 in which the surgical probe comprises:

one or more handling control surfaces configured to permit a user to extend an entirety of the first cavity into an organism.

73. The medical or veterinary system of clause 44 in which the surgical probe comprises:

a first separable extraction module containing the first cavity and supportable by and separable from a remainder of the surgical instrument.

74. The medical or veterinary system of clause 44, further comprising:

one or more media bearing an attribute of a macromolecule of the tissue sample.

75. The medical or veterinary system of clause 44, further comprising:

one or more media bearing a morphological category relating to a portion of the tissue sample.

76. The medical or veterinary system of clause 44, further comprising:

circuitry for selecting a stain effective for indicating whether the tissue sample includes a chromosomal abnormality.

77. The medical or veterinary system of clause 44, further comprising:

circuitry for causing the tissue sample to come into contact with a stain effective for indicating whether the tissue sample apparently exhibits an attribute of interest.

78. The medical or veterinary system of clause 44 in which a portion of the first treatment module comprises:

a dispenser operable to mark a portion of the tissue sample with a luminescent marking agent of the first treatment.

79. The medical or veterinary system of clause 44 in which a portion of the first treatment module comprises:

a dispenser operable to mark a portion of the tissue sample with a stain of the first treatment.

80. The medical or veterinary system of clause 44, further comprising:

circuitry for configuring the result to include one or more size-descriptive quantities relating to the tissue sample.

81. A medical or veterinary system comprising:

a device having at least (a) a handling control surface, (b) a distal portion narrow enough to extend into a living organism, (c) a first dispenser configured to apply a first treatment material to tissue adjacent the device, and (d) a first output module configured to transmit a result of the first treatment material.

82. The medical or veterinary system of clause 81 in which the first treatment module comprises:

circuitry for actuating the first dispenser in response to contemporaneous user input.

83. The medical or veterinary system of clause 81, further comprising:

circuitry for positioning at least the distal portion of the device.

84. The medical or veterinary system of clause 81, further comprising:

circuitry for processing data from one or more assay protocols performed upon a sample of the tissue.

85. The medical or veterinary system of clause 81, further comprising:

circuitry for processing data from one or more biomarker detection protocols performed upon a sample of the tissue.

86. The medical or veterinary system of clause 81, further comprising:

at least one medium bearing an operational setting value usable in laser scanning equipment for analyzing a sample of the tissue.

87. The medical or veterinary system of clause 81, further comprising:

at least one medium bearing an operational setting value usable in a mass spectroscope for analyzing a sample of the tissue.

88. The medical or veterinary system of clause 81, further comprising:

at least one medium bearing an operational setting value usable in a microscope for analyzing a sample of the tissue.

89. The medical or veterinary system of clause 81, further comprising:

laser scanning equipment operable for receiving at least the distal portion of the device.

90. The medical or veterinary system of clause 81, further comprising:

a separable extraction module of the device; and

imaging equipment operable for receiving the separable extraction module of the device.

91. The medical or veterinary system of clause 81, further comprising:

a recessed portion of the device containing a sample of the tissue adjacent the device.

92. The medical or veterinary system of clause 81, further comprising:

a recessed portion of the device containing a sample of the tissue adjacent the device; and

one or more conduits operably coupling the first output module with imaging equipment operable to detect the result of the first treatment.

93. The medical or veterinary system of clause 81, further comprising:

circuitry for configuring the result to include one or more quantifications derived from an optical field of the tissue adjacent the device.

94. The medical or veterinary system of clause 81 in which the first treatment material comprises:

a drug.

95. The medical or veterinary system of clause 81 in which the first treatment material comprises:

a buffer.

96. The medical or veterinary system of clause 81 in which the first treatment material comprises:

a permeabilizing agent.

97. The medical or veterinary system of clause 81 in which the device further comprises:

an electropermeabilization module configured to permeabilize at least some of the tissue.

98. The medical or veterinary system of clause 81, further comprising:

a device configured to extract a sample of the tissue adjacent the distal portion.

99. The medical or veterinary system of clause 81, in which the output module comprises:

a component configured to observe the tissue and to transmit the result via one or more conduits.

100. The medical or veterinary system of clause 81, in which the output module comprises:

a component configured to observe the tissue adjacent the device and to cause at least some of the result of the first treatment material to be stored.

101. The medical or veterinary system of clause 81, in which the output module comprises:

a sensor configured to observe the tissue adjacent the device and to transmit at least some of the result on one or more physical media.

102. The medical or veterinary system of clause 81, further comprising:

an electron microscope configured to observe the tissue adjacent the device and to provide at least some of the result on one or more physical media.

103. The medical or veterinary system of clause 81, further comprising:

a fluorescence microscope configured to observe the tissue adjacent the device and to provide at least some of the result on one or more physical media.

104. The medical or veterinary system of clause 81, further comprising:

a confocal microscope configured to observe the tissue adjacent the device and to provide at least some of the result on one or more physical media.

105. The medical or veterinary system of clause 81, further comprising:

a spectrometer configured to observe the tissue adjacent the device and to provide at least some of the result on one or more physical media.

106. The medical or veterinary system of clause 81, further comprising:

an imaging system configured to observe the tissue adjacent the device and to provide at least some of the result on one or more physical media.

107. The medical or veterinary system of clause 81, further comprising:

a nuclear magnetic resonance imaging system configured to observe the tissue adjacent the device and to provide at least some of the result on one or more physical media.

108. The medical or veterinary system of clause 81, further comprising:

circuitry for transmitting energy into a sample of the tissue adjacent the device; and

circuitry for capturing an image of the sample of the tissue.

109. The medical or veterinary system of clause 81 in which the device further comprises:

the handling control surface configured to permit a user to extend an entirety of a first tissue extraction cavity into the living organism.

110. The medical or veterinary system of clause 81 in which the device further comprises:

a first separable extraction module containing a cavity and supportable by and separable from a remainder of the device.

111. The medical or veterinary system of clause 81, further comprising:

one or more media bearing an attribute of a macromolecule relating to the tissue adjacent the device.

112. The medical or veterinary system of clause 81, further comprising:

one or more media bearing a morphological category relating to a portion of the tissue adjacent the device.

113. The medical or veterinary system of clause 81, further comprising:

circuitry for causing the tissue adjacent the device to come into contact with a stain effective for indicating whether the tissue apparently exhibits an attribute of interest.

114. The medical or veterinary system of clause 81 in which the first dispenser comprises:

a fluorescent marking agent of the first treatment material.

115. The medical or veterinary system of clause 81 in which the first dispenser comprises:

a stain of the first treatment material.

116. The medical or veterinary system of clause 81, further comprising:

a second dispenser operable to administer a stain.

117. The medical or veterinary system of clause 81, further comprising:

circuitry for configuring the result to include one or more size-descriptive quantities relating to the tissue adjacent the device.

118. A medical or veterinary system comprising:

a first dispenser configured to apply a first treatment material to tissue of an organism in vivo;

a cooling component configured to freeze at least some of the tissue in vivo; and

an extraction element configured to remove at least a portion of the tissue from the organism.

119. The medical or veterinary system of clause 118, further comprising:

circuitry for actuating the first dispenser in response to contemporaneous user input.

120. The medical or veterinary system of clause 118, further comprising:

circuitry for positioning at least a portion of the first dispenser.

121. The medical or veterinary system of clause 118, further comprising:

circuitry for processing data from one or more assay protocols performed upon the portion of the tissue.

122. The medical or veterinary system of clause 118, further comprising:

circuitry for processing data from one or more biomarker detection protocols performed upon (at least some of) the portion of the tissue.

123. The medical or veterinary system of clause 118, further comprising:

at least one medium bearing an operational setting value usable in laser scanning equipment for analyzing the portion of the tissue.

124. The medical or veterinary system of clause 118, further comprising:

at least one medium bearing an operational setting value usable in a mass spectroscope for analyzing the portion of the tissue.

125. The medical or veterinary system of clause 118, further comprising:

at least one medium bearing an operational setting value usable in a microscope for analyzing the portion of the tissue.

126. The medical or veterinary system of clause 118, further comprising:

laser scanning equipment operable for receiving at least some of the extraction element of the device.

127. The medical or veterinary system of clause 118, further comprising:

imaging equipment operable for receiving at least some of the extraction element of the device.

128. The medical or veterinary system of clause 118 in which the extraction element comprises:

a recessed portion configured to contain the portion of the tissue.

129. The medical or veterinary system of clause 118, further comprising:

one or more conduits operably coupling imaging equipment with a portion of the extraction element configured to support the portion of the tissue.

130. The medical or veterinary system of clause 118 in which the first treatment material comprises:

a drug.

131. The medical or veterinary system of clause 118 in which the first treatment material comprises:

a buffer.

132. The medical or veterinary system of clause 118 in which the first treatment material comprises:

a permeabilizing agent.

133. The medical or veterinary system of clause 118, further comprising:

a microinjection module configured to penetrate at least one cell of the tissue in vivo.

134. The medical or veterinary system of clause 118, further comprising:

an electropermeabilization module configured to permeabilize at least one cell of the tissue in vivo.

135. The medical or veterinary system of clause 118, further comprising:

circuitry for generating one or more quantifications from an optical field of the tissue from the organism.

136. The medical or veterinary system of clause 118, in which the extraction element comprises:

a microtome configured to section the portion of the tissue.

137. The medical or veterinary system of clause 118, in which the extraction element comprises:

a device configured to extract the portion of the tissue by dividing a sample of the tissue.

138. The medical or veterinary system of clause 118, in which the extraction element comprises:

a component configured to observe the portion of the tissue in a first cavity and to transmit an output via one or more conduits.

139. The medical or veterinary system of clause 118, in which the extraction element comprises:

circuitry for causing a result of the first treatment material to be stored on one or more physical media.

140. The medical or veterinary system of clause 118, further comprising:

circuitry for observing the tissue configured to cause a presentation of an output on one or more physical media.

141. The medical or veterinary system of clause 118, further comprising:

circuitry for transmitting a go/no-go result of the first treatment material on one or more physical media.

142. The medical or veterinary system of clause 118, further comprising:

an electron microscope configured to observe the portion of the tissue and to provide an output on one or more physical media.

143. The medical or veterinary system of clause 118, further comprising:

a fluorescence microscope configured to observe the portion of the tissue and to provide an output on one or more physical media.

144. The medical or veterinary system of clause 118, further comprising:

a confocal microscope configured to observe the portion of the tissue and to provide an output on one or more physical media.

145. The medical or veterinary system of clause 118, further comprising:

a spectrometer configured to observe the portion of the tissue and to provide an output on one or more physical media.

146. The medical or veterinary system of clause 118, further comprising:

an imaging system configured to observe the portion of the tissue and to provide an output on one or more physical media.

147. The medical or veterinary system of clause 118, further comprising:

a nuclear magnetic resonance imaging system configured to observe the portion of the tissue and to provide an output on one or more physical media.

148. The medical or veterinary system of clause 118, further comprising:

circuitry for transmitting energy into the portion of the tissue; and

circuitry for capturing an image of the portion of the tissue.

149. The medical or veterinary system of clause 118, further comprising:

one or more handling control surfaces configured to permit a user to extend an entirety of the extraction element into the organism.

150. The medical or veterinary system of clause 118, in which the extraction element comprises:

a first separable module containing a cavity and supportable by and separable from a remainder of the extraction element.

151. The medical or veterinary system of clause 118, further comprising:

one or more media bearing an attribute of a macromolecule relating to the tissue.

152. The medical or veterinary system of clause 118, further comprising:

one or more media bearing a morphological category relating to a portion of the tissue of the organism.

153. The medical or veterinary system of clause 118, further comprising:

circuitry for selecting a stain effective for indicating whether the tissue includes a chromosomal abnormality.

154. The medical or veterinary system of clause 118, further comprising:

circuitry for causing the portion of the tissue to come into contact with a stain effective for indicating whether the tissue apparently exhibits an attribute of interest.

155. The medical or veterinary system of clause 118 in which the first dispenser comprises:

a permeabilizing agent of the first treatment material.

156. The medical or veterinary system of clause 118 in which the first dispenser comprises:

a luminescent marking agent of the first treatment material.

157. The medical or veterinary system of clause 118 in which the first dispenser comprises:

a stain of the first treatment material.

158. The medical or veterinary system of clause 118, further comprising:

circuitry for configuring a result of the first treatment material to include one or more size-descriptive quantities relating to the tissue.

159. The medical or veterinary system of clause 118, further comprising:

circuitry for generating one or more size-descriptive quantities relating to the portion of the tissue from the organism.

160. A medical or veterinary system comprising:

a probe having at least a first dispenser configured to apply a first treatment material to tissue of an organism in vivo, a first optical element configured to transmit light into the tissue of the organism in vivo, and a first output module configured to transmit a result of at least the light and the first treatment material upon the tissue of the organism in vivo.

161. The medical or veterinary system of clause 160, further comprising:

circuitry for actuating the first dispenser in response to contemporaneous user input.

162. The medical or veterinary system of clause 160, further comprising:

circuitry for positioning at least a portion of the first dispenser.

163. The medical or veterinary system of clause 160, further comprising:

circuitry for processing data from one or more assay protocols performed upon a portion of the tissue.

164. The medical or veterinary system of clause 160, further comprising:

circuitry for processing data from one or more biomarker detection protocols performed upon a portion of the tissue.

165. The medical or veterinary system of clause 160, further comprising:

at least one medium bearing an operational setting value usable in laser scanning equipment for analyzing a portion of the tissue.

166. The medical or veterinary system of clause 160, further comprising:

at least one medium bearing an operational setting value usable in a mass spectroscope for analyzing a portion of the tissue.

167. The medical or veterinary system of clause 160, further comprising:

at least one medium bearing an operational setting value usable in a microscope for analyzing a portion of the tissue.

168. The medical or veterinary system of clause 160, further comprising:

laser scanning equipment operable for receiving a portion of the probe configured to contain a sample of the tissue.

169. The medical or veterinary system of clause 160, in which the probe further comprises:

an extraction module configured to contain a sample of the tissue.

170. The medical or veterinary system of clause 160, in which the first optical element comprises:

an infrared emitter.

171. The medical or veterinary system of clause 160, in which the first optical element comprises:

a conduit configured (at least) to bear the light into the tissue of the organism.

172. The medical or veterinary system of clause 160, in which the first output module comprises:

a conduit configured (at least) to bear the result.

173. The medical or veterinary system of clause 160, further comprising:

imaging equipment operable for receiving an extraction module of the probe.

174. The medical or veterinary system of clause 160 in which the extraction element comprises:

a recessed portion configured to contain a sample of the tissue.

175. The medical or veterinary system of clause 160, further comprising:

one or more conduits operably coupling imaging equipment with a portion of the probe configured to contain a portion of the tissue.

176. The medical or veterinary system of clause 160 in which the first treatment material comprises:

a drug.

177. The medical or veterinary system of clause 160 in which the first treatment material comprises:

a buffer.

178. The medical or veterinary system of clause 160 in which the first treatment material comprises:

a permeabilizing agent.

179. The medical or veterinary system of clause 160 in which the first treatment material comprises:

a fixative.

180. The medical or veterinary system of clause 160 in which the first treatment material comprises:

a stain.

181. The medical or veterinary system of clause 160 in which the first treatment material comprises:

an antibody.

182. The medical or veterinary system of clause 160, further comprising:

circuitry for configuring the result to include one or more quantifications derived from an optical field of the tissue.

183. The medical or veterinary system of clause 160, further comprising:

a laser microtome configured to section a sample of the tissue.

184. The medical or veterinary system of clause 160, in which the output module comprises:

a device configured to observe the tissue in a first cavity and to transmit via a conduit the result of at least the light and the first treatment material upon the tissue of the organism in vivo.

185. The medical or veterinary system of clause 160, in which the output module comprises:

circuitry for causing at least some of the result to be stored.

186. The medical or veterinary system of clause 160, further comprising:

circuitry for causing a presentation of at least a go/no-go component of the result on one or more physical media.

187. The medical or veterinary system of clause 160, further comprising:

an electron microscope configured to observe the tissue and to provide at least some of the result on one or more physical media.

188. The medical or veterinary system of clause 160, further comprising:

a fluorescence microscope configured to observe the tissue and to provide at least some of the result on one or more physical media.

189. The medical or veterinary system of clause 160, further comprising:

a confocal microscope (at least) configured to observe (at least) a sample of (at least) the tissue and to provide at least some of the result on (at least) a physical medium.

190. The medical or veterinary system of clause 160, further comprising:

a spectrometer configured to observe the tissue and to provide at least some of the result on one or more physical media.

191. The medical or veterinary system of clause 160, further comprising:

an imaging system configured to observe the tissue and to provide at least some of the result on one or more physical media.

192. The medical or veterinary system of clause 160, further comprising:

a nuclear magnetic resonance imaging system configured to observe the tissue and to provide at least some of the result on one or more physical media.

193. The medical or veterinary system of clause 160, further comprising:

circuitry for transmitting energy into a sample of the tissue; and

circuitry for capturing an image of the sample of the tissue.

194. The medical or veterinary system of clause 160 in which the surgical probe comprises:

one or more handling control surfaces configured to permit a user to extend an entirety of a first cavity into the organism.

195. The medical or veterinary system of clause 160 in which the surgical probe comprises:

a first separable extraction module containing a cavity and supportable by and separable from a remainder of the probe.

196. The medical or veterinary system of clause 160, further comprising:

one or more media bearing an attribute of a macromolecule relating to the tissue of the organism.

197. The medical or veterinary system of clause 160, further comprising:

one or more media bearing a morphological category relating to a portion of the tissue of the organism.

198. The medical or veterinary system of clause 160, further comprising:

circuitry for causing a sample of the tissue to come into contact with a stain effective for indicating whether the tissue apparently exhibits an attribute of interest.

199. The medical or veterinary system of clause 160, in which the first dispenser comprises:

a stain effective for indicating whether the tissue apparently exhibits a chromosomal attribute of interest.

200. The medical or veterinary system of clause 160, in which the first dispenser comprises:

a luminescent marking agent of the first treatment material.

201. The medical or veterinary system of clause 160, in which the first dispenser comprises:

a stain of the first treatment material.

202. The medical or veterinary system of clause 160, further comprising:

a second dispenser configured to apply a second material containing at least a fixative.

203. The medical or veterinary system of clause 160, further comprising:

circuitry for configuring the result to include one or more size-descriptive quantities relating to the tissue.

204. An apparatus comprising:

one or more physical media bearing device-detectable data indicating an extraction of chemically treated tissue frozen in vivo.

205. The apparatus of clause 204 in which the device-detectable data comprises:

one or more identifiers of a protocol by which the tissue was frozen before the extraction.

206. The apparatus of clause 204 in which the device-detectable data comprises:

one or more identifiers of a protocol by which the tissue was chemically treated.

207. The apparatus of clause 204 in which the device-detectable data comprises:

one or more operational parameters of a protocol by which the tissue was chemically treated.

208. The apparatus of clause 204 in which the device-detectable data comprises:

one or more identifiers of a material by which the tissue was chemically treated.

209. The apparatus of clause 204 in which the device-detectable data comprises:

one or more identifiers of a stain by which the tissue was chemically treated.

210. The apparatus of clause 204 in which the device-detectable data comprises:

a go/no-go indication relating to the chemically treated tissue frozen in vivo.

211. The apparatus of clause 204 in which the device-detectable data comprises:

a laser-scanned image of at least some of a cell to which an optical enhancement material was applied in vivo.

212. The apparatus of clause 204 in which the device-detectable data comprises:

an earlier image depicting at least some of a cell before the cell freezes; and

a later image depicting at least some of the cell after the cell freezes.

213. The apparatus of clause 204 in which the device-detectable data comprises:

an earlier image depicting at least some of a cell before the cell freezes; and

a later image depicting at least some of the cell before the cell freezes.

214. The apparatus of clause 204 in which the one or more physical media comprises:

some of the one or more physical media bearing a component of the device-detectable data indicating an optical treatment in a chamber extended into an organism.

215. The apparatus of clause 204, further comprising:

circuitry for causing the chemically treated tissue to be frozen in vivo in response to contemporaneous user input.

216. The apparatus of clause 204, further comprising:

circuitry for positioning a dispenser adjacent the tissue.

217. The apparatus of clause 204, further comprising:

circuitry for processing a component of the device-detectable data obtained from one or more assay protocols performed upon at least some of the tissue.

218. The apparatus of clause 204, further comprising:

circuitry for processing a component of the device-detectable data obtained from one or more biomarker detection protocols performed upon at least some of the tissue.

219. The apparatus of clause 204, further comprising:

circuitry for processing a component of the device-detectable data obtained from one or more laser scanning protocols performed upon at least some of the tissue.

220. The apparatus of clause 204, further comprising:

one or more other physical media bearing an operational setting value usable in laser scanning equipment for analyzing a portion of the tissue.

221. The apparatus of clause 204 in which the one or more physical media comprises:

at least one of the one or more physical media bearing an operational setting value usable for analyzing a portion of the tissue.

222. The apparatus of clause 204 in which the one or more physical media comprises:

at least one of the one or more physical media having borne an operational setting value usable in a microscope for analyzing a portion of the tissue.

223. The apparatus of clause 204 in which the one or more physical media comprises:

a conduit configured to bear a result of an optical treatment in vivo upon the chemically treated tissue.

224. The apparatus of clause 204 in which the one or more physical media comprises:

a conduit configured to bear a result of an optical treatment upon the chemically treated tissue frozen in vivo.

225. The apparatus of clause 204 in which the one or more physical media comprises:

one or more conduits coupling imaging equipment with a module configured to contain the extraction.

226. The apparatus of clause 204 in which the one or more physical media comprises:

one or more conduits coupling imaging equipment with an instrument configured to perform the extraction.

227. The apparatus of clause 204 in which the extraction comprises:

at least an unfrozen portion of the tissue.

228. The apparatus of clause 204 in which the extraction comprises:

at least some of the tissue that was chemically treated with a drug.

229. The apparatus of clause 204 in which the extraction comprises:

at least some of the tissue that was chemically treated with an aptamer.

230. The apparatus of clause 204 in which the extraction comprises:

at least some of the tissue that was chemically treated with a permeabilizing agent.

231. The apparatus of clause 204 in which a portion of one or more physical media comprises:

one or more quantifications derived from an optical field of the chemically treated tissue.

232. The apparatus of clause 204 in which a portion of the one or more physical media comprises:

an attribute of a macromolecule relating to the tissue.

233. The apparatus of clause 204 in which a portion of the one or more physical media comprises:

a shape-indicative category relating to a portion of the tissue.

234. The apparatus of clause 204 in which a portion of the one or more physical media comprises:

an indication of whether the tissue apparently exhibits a chromosomal attribute of interest.

235. The apparatus of clause 204 in which a portion of the one or more physical media comprises:

an indication of how much of the tissue apparently exhibits a pathology.

236. The apparatus of clause 204 in which a portion of the one or more physical media comprises:

a portion of the device-detectable data indicating that the tissue was chemically treated with a luminescent marking agent.

237. The apparatus of clause 204 in which a portion of the one or more physical media comprises:

a portion of the device-detectable data indicating that the tissue was chemically treated with a stain.

238. The apparatus of clause 204 in which a portion of the one or more physical media comprises:

one or more size-descriptive quantities relating to the chemically treated tissue.

239. The apparatus of clause 204, further comprising:

a dispenser configured to inject a stain into a part of a living organism;

a freezing element configured to freeze at least some of the part in vivo of the living organism; and

circuitry for indicating the extraction on at least one of the one or more physical media.

240. The apparatus of clause 204 in which the one or more physical media comprise:

a portable module including at least an auditory interface configured to be operated while the portable module is held or worn.

241. The apparatus of clause 204 in which a portion of the one or more physical media comprises:

an image projection module.

242. The apparatus of clause 204 in which a portion of the one or more physical media comprises:

a touch screen.

243. The apparatus of clause 204 in which the one or more physical media include at least one of a repeater, a communication satellite, or another active module configured to accept first and second portions of the device-detectable data at first and second respective times.

244. The apparatus of clause 204 in which a portion of the one or more physical media comprises:

one or more processors configured to perform one or more of optical image scanning or auditory pattern scanning upon the device-detectable data.

245. The apparatus of clause 204 in which a portion of the one or more physical media comprises:

one or more processors configured to perform linguistic pattern scanning upon the device-detectable data.

246. The apparatus of clause 204 in which a portion of the one or more physical media comprises:

circuitry for using an encryption constraint in at least some of the device-detectable data.

247. The apparatus of clause 204 in which at least one of the one or more physical media comprises:

one or more signal-bearing media bearing at least one of a special-purpose instruction sequence or an information-bearing static attribute as a portion of the device-detectable data.

248. The apparatus of clause 204 in which a first portion of the one or more physical media transmits a portion of the device-detectable data before a remainder of the one or more physical media transmits a remainder of the device-detectable data.

249. The apparatus of clause 204 in which the one or more physical media include at least one of an integrated circuit, a data-holding element, a lens or other light-transmissive medium, a signal-bearing conduit currently bearing at least a portion of the device-detectable data, or a bus or other configuration of two or more transmission media in mutual isolation.

250. The apparatus of clause 204 in which a portion of the one or more physical media comprises:

a power line operated for transmitting content of the device-detectable data between at least two terminals.

251. The apparatus of clause 204 in which a first medium of the one or more physical media bears a first portion of the device-detectable data while a second medium of the one or more physical media bears a second portion of the device-detectable data.

252. The apparatus of clause 204 in which the one or more physical media are configured at least (a) by causing a communication channel in the one or more physical media to bear a first portion of the device-detectable data; and (b) by causing another channel of the one or more physical media to bear a second portion of the device-detectable data.

253. The apparatus of clause 204 in which the one or more physical media have borne the device-detectable data.

254. The apparatus of clause 204 in which a portion of the one or more physical media comprises:

one or more static markings indicative of the device-detectable data.

255. The apparatus of clause 204 in which a portion of the one or more physical media comprises:

a magnetoresistive random access memory configured to receive the device-detectable data.

256. The apparatus of clause 204 further comprising at least one of a satellite dish or other signal-reflective element, a transducer, an antenna, or a receiver operated to receive the device-detectable data.

257. An apparatus comprising:

one or more physical media bearing device-detectable data indicating a treatment of a tissue component in a chamber extended into tissue of an organism.

258. The apparatus of clause 257, further comprising:

a device containing the chamber, positioned with a handling control surface.

259. The apparatus of clause 257 in which the device-detectable data indicating the treatment of the tissue component in the chamber comprises:

output from a device positioned with a handling control surface.

260. The apparatus of clause 257 in which the device-detectable data indicating the treatment of the tissue component in the chamber comprises:

a product of a noninvasive protocol.

261. The apparatus of clause 257 in which the device-detectable data indicating the treatment of the tissue component in the chamber comprises:

a product of a minimally invasive protocol.

262. The apparatus of clause 257 in which the device-detectable data indicating the treatment of the tissue component in the chamber comprises:

a product of a surgical protocol.

263. The apparatus of clause 257 in which the device-detectable data indicating the treatment of the tissue component in the chamber comprises:

a product of an agricultural protocol.

264. The apparatus of clause 257 in which the device-detectable data indicating the treatment of the tissue component in the chamber comprises:

image data depicting a cell of the tissue component.

265. The apparatus of clause 257 in which the device-detectable data indicating the treatment of the tissue component in the chamber comprises:

image data depicting frozen tissue including the tissue component.

266. The apparatus of clause 257 in which the device-detectable data indicating the treatment of the tissue component in the chamber comprises:

a signal from a probe that was positioned adjacent the tissue component in vivo.

267. The apparatus of clause 257 in which the device-detectable data indicating the treatment of the tissue component in the chamber comprises:

a signal from a surgical instrument that was positioned adjacent the tissue component.

268. The apparatus of clause 257 in which the one or more physical media further comprises:

an image of the tissue component from an electron microscope.

269. The apparatus of clause 257 in which the one or more physical media further comprises:

an image of the tissue component from laser-scanning equipment.

270. The apparatus of clause 257 in which the one or more physical media further comprises:

some of the one or more physical media bearing a signal from a biosensor.

271. The apparatus of clause 257 in which the one or more physical media further comprises:

a result of an in situ hybridization protocol performed upon some of the tissue component.

272. The apparatus of clause 257 in which the one or more physical media further comprises:

a result of positioning at least some of the tissue component in a microfluidic structure.

273. The apparatus of clause 257 in which the one or more physical media further comprises:

some of the one or more physical media bearing a result of the treatment including one or more antibodies.

274. The apparatus of clause 257 in which the one or more physical media further comprises:

a result of material applied in vivo indicating an absence of or a presence of a first attribute in the tissue component

275. The apparatus of clause 257 in which the one or more physical media further comprises:

some of the one or more physical media bearing a portion of the device-detectable data received from one or more chemical sensors.

276. The apparatus of clause 257 in which the device-detectable data indicating the treatment of the tissue component in the chamber comprises:

a karyotype of the organism.

277. The apparatus of clause 257 in which the device-detectable data indicating the treatment of the tissue component in the chamber comprises:

a data component relating to blood extracted from the organism.

278. The apparatus of clause 257 in which the device-detectable data indicating the treatment of the tissue component in the chamber comprises:

a data component relating to fluid extracted from the organism.

279. The apparatus of clause 257 in which the device-detectable data indicating the treatment of the tissue component in the chamber comprises:

an extraction protocol descriptor.

280. The apparatus of clause 257 in which the device-detectable data comprises:

one or more identifiers of a protocol by which the tissue component was treated in the chamber.

281. The apparatus of clause 257 in which the device-detectable data comprises:

one or more identifiers of a protocol by which the tissue component was frozen.

282. The apparatus of clause 257 in which the device-detectable data comprises:

one or more identifiers of a protocol by which the tissue component was optically treated.

283. The apparatus of clause 257 in which the device-detectable data comprises:

one or more identifiers of an agent to which the tissue component was exposed in the chamber.

284. The apparatus of clause 257 in which the device-detectable data comprises:

one or more identifiers of a marking agent by which the tissue component was chemically treated.

285. The apparatus of clause 257 in which the device-detectable data comprises:

a go/no-go indication relating to the tissue component.

286. The apparatus of clause 257 in which the device-detectable data comprises:

a go/no-go indication of an extraction of the tissue component.

287. The apparatus of clause 257 in which the device-detectable data comprises:

a laser-scanned image of at least some of a cell to which an optical enhancement material of the treatment was applied in vivo.

288. The apparatus of clause 257 in which the device-detectable data comprises:

an earlier image depicting the tissue component unfrozen; and

a later image depicting the tissue component frozen.

289. The apparatus of clause 257 in which the one or more physical media comprises:

some of the one or more physical media bearing a component of the device-detectable data indicating an optical treatment of the tissue component.

290. The apparatus of clause 257 in which the one or more physical media comprises:

some of the one or more physical media bearing a component of the device-detectable data indicating a chemical component of the treatment of the tissue component in the chamber.

291. The apparatus of clause 257 in which the one or more physical media comprises:

some of the one or more physical media bearing a component of the device-detectable data indicating the treatment of the tissue component in the chamber.

292. The apparatus of clause 257 in which the one or more physical media comprises:

some of the one or more physical media bearing a component of the device-detectable data indicating the treatment of the tissue component in the chamber after separating the chamber from the tissue of the organism.

293. The apparatus of clause 257, further comprising:

circuitry for causing chemically treated tissue to be frozen in vivo in response to contemporaneous user input.

294. The apparatus of clause 257, further comprising:

circuitry for positioning a dispenser adjacent the tissue.

295. The apparatus of clause 257, further comprising:

circuitry for processing a component of the device-detectable data obtained from one or more assay protocols performed upon at least some of the tissue.

296. The apparatus of clause 257, further comprising:

circuitry for processing a component of the device-detectable data obtained from one or more biomarker detection protocols performed upon at least some of the tissue.

297. The apparatus of clause 257, further comprising:

circuitry for processing a component of the device-detectable data obtained from one or more laser scanning protocols performed upon at least some of the tissue.

298. The apparatus of clause 257, further comprising:

one or more other physical media bearing an operational setting value usable in laser scanning equipment for analyzing a portion of the tissue.

299. The apparatus of clause 257 in which the one or more physical media comprises:

at least one of the one or more physical media bearing an operational setting value usable for analyzing a portion of the tissue.

300. The apparatus of clause 257 in which the one or more physical media comprises:

at least one of the one or more physical media having borne an operational setting value usable in a microscope for analyzing a portion of the tissue.

301. The apparatus of clause 257 in which the one or more physical media comprises:

a conduit configured to bear a result of an optical treatment in vivo upon the chemically treated tissue.

302. The apparatus of clause 257 in which the one or more physical media comprises:

a conduit configured to bear a result of an optical treatment upon the chemically treated tissue frozen in vivo.

303. The apparatus of clause 257 in which the one or more physical media comprises:

one or more conduits coupling imaging equipment with a module configured to contain the extraction.

304. The apparatus of clause 257 in which the one or more physical media comprises:

one or more conduits coupling imaging equipment with an instrument configured to perform the extraction.

305. The apparatus of clause 257 in which the treatment comprises:

alcohol.

306. The apparatus of clause 257 in which the treatment comprises:

buffered formalin.

307. The apparatus of clause 257 in which the treatment comprises:

a permeabilizing agent.

308. The apparatus of clause 257 in which the treatment comprises:

one or more of a microinjection or an electropermeabilization.

309. The apparatus of clause 257 in which a portion of the one or more physical media comprises:

one or more quantifications derived from an optical field of the tissue component.

310. The apparatus of clause 257 in which a portion of the one or more physical media comprises:

an attribute of a macromolecule relating to the tissue.

311. The apparatus of clause 257 in which a portion of the one or more physical media comprises:

an indication of whether the tissue apparently exhibits a chromosomal attribute of interest.

312. The apparatus of clause 257 in which a portion of the one or more physical media comprises:

an indication of how much of the tissue apparently exhibits a pathology.

313. The apparatus of clause 257 in which a portion of the one or more physical media comprises:

a portion of the device-detectable data indicating a luminescent marking agent in the treatment of the tissue component.

314. The apparatus of clause 257 in which a portion of the one or more physical media comprises:

a portion of the device-detectable data indicating a stain in the treatment of the tissue component.

315. The apparatus of clause 257 in which a portion of the one or more physical media comprises:

one or more size-descriptive quantities relating to the tissue component.

316. The apparatus of clause 257 in which the one or more physical media comprise:

at least one of the one or more physical media bearing a component of the device-detectable data that was generated while the treatment was applied to the tissue component.

317. The apparatus of clause 257 in which the one or more physical media comprise:

at least one of the one or more physical media bearing a component of the device-detectable data that was generated after the chamber was withdrawn from the tissue of the organism.

318. The apparatus of clause 257 in which the one or more physical media comprise:

at least one of the one or more physical media bearing a result component of the device-detectable data that was generated from raw sensor data.

319. The apparatus of clause 257 in which the one or more physical media comprise:

at least one of the one or more physical media bearing a component of the device-detectable data that was generated while the chamber extended into the tissue of the organism.

320. The apparatus of clause 257, further comprising:

an extraction module containing the chamber, in which the treatment commenced upon a portion of the tissue in the chamber and continued upon the tissue component in the chamber.

321. The apparatus of clause 257, further comprising:

an extraction module containing the chamber, in which the chamber contained a reagent to begin the treatment upon a portion of the tissue entering the chamber.

322. The apparatus of clause 257, further comprising:

a laser microtome configured to extract the tissue component by severing a portion of the tissue in the chamber from a remainder of the tissue.

323. The apparatus of clause 257, further comprising:

an instrument configured to observe the tissue component in the chamber and to transmit at least some of the device-detectable data on the one or more physical media.

324. The apparatus of clause 257, further comprising:

an instrument configured to observe the tissue component in the chamber and to store at least some of the device-detectable data on the one or more physical media.

325. The apparatus of clause 257, further comprising:

an instrument configured to observe the tissue component in the chamber and to present at least some of the device-detectable data on the one or more physical media.

326. The apparatus of clause 257, further comprising:

an electron microscope configured to observe the tissue component in the chamber and to provide at least some of the device-detectable data on the one or more physical media.

327. The apparatus of clause 257, further comprising:

a fluorescence microscope configured to observe the tissue component in the chamber and to provide at least some of the device-detectable data on the one or more physical media.

328. The apparatus of clause 257, further comprising:

a confocal microscope configured to observe the tissue component in the chamber and to provide at least some of the device-detectable data on the one or more physical media.

329. The apparatus of clause 257, further comprising:

a spectrometer configured to observe the tissue component in the chamber and to provide at least some of the device-detectable data on the one or more physical media.

330. The apparatus of clause 257, further comprising:

an imaging system configured to observe the tissue component in the chamber and to provide at least some of the device-detectable data on the one or more physical media.

331. The apparatus of clause 257, further comprising:

a nuclear magnetic resonance imaging system configured to observe the tissue component in the chamber and to provide at least some of the device-detectable data on the one or more physical media.

332. The apparatus of clause 257, further comprising:

circuitry for transmitting energy into the tissue component in the chamber; and

circuitry for capturing an image of the tissue component.

333. The apparatus of clause 257, further comprising:

a surgical instrument with a handling control surface; and

an extraction module containing the chamber and supportable by and separable from the surgical instrument.

334. The apparatus of clause 257, further comprising:

a handling control surface configured to permit a user to extend an entirety of the chamber into the organism.

335. The apparatus of clause 257, further comprising:

at least one of the one or more physical media bearing a descriptor of an instrument that contains the chamber.

336. The apparatus of clause 257, further comprising:

the device-detectable data indicating at least a therapeutic agent used in the treatment of the tissue component.

337. The apparatus of clause 257, further comprising:

the device-detectable data indicating at least a therapeutic agent administered to the tissue in vivo.

338. The apparatus of clause 257 in which the one or more physical media comprise:

a portable module including at least an auditory interface configured to be operated while the portable module is held or worn.

339. The apparatus of clause 257 in which a portion of the one or more physical media comprises:

an image projection module.

340. The apparatus of clause 257 in which a portion of the one or more physical media comprises:

a touch screen.

341. The apparatus of clause 257 in which the one or more physical media include at least one of a repeater, a communication satellite, or another active module configured to accept first and second portions of the device-detectable data at first and second respective times.

342. The apparatus of clause 257 in which a portion of the one or more physical media comprises:

one or more processors configured to perform one or more of optical image scanning or auditory pattern scanning upon the device-detectable data.

343. The apparatus of clause 257 in which a portion of the one or more physical media comprises:

one or more processors configured to perform linguistic pattern scanning upon the device-detectable data.

344. The apparatus of clause 257 in which a portion of the one or more physical media comprises:

circuitry for using an encryption constraint in at least some of the device-detectable data.

345. The apparatus of clause 257 in which at least one of the one or more physical media comprises:

one or more signal-bearing media bearing at least one of a special-purpose instruction sequence or an information-bearing static attribute as a portion of the device-detectable data.

346. The apparatus of clause 257 in which a first portion of the one or more physical media transmits a portion of the device-detectable data before a remainder of the one or more physical media transmits a remainder of the device-detectable data.

347. The apparatus of clause 257 in which the one or more physical media include at least one of an integrated circuit, a data-holding element, a lens or other light-transmissive medium, a signal-bearing conduit currently bearing at least a portion of the device-detectable data, or a bus or other configuration of two or more transmission media in mutual isolation.

348. The apparatus of clause 257 in which a portion of the one or more physical media comprises:

a power line operated for transmitting content of the device-detectable data between at least two terminals.

349. The apparatus of clause 257 in which a first medium of the one or more physical media bears a first portion of the device-detectable data while a second medium of the one or more physical media bears a second portion of the device-detectable data.

350. The apparatus of clause 257 in which the one or more physical media are configured at least (a) by causing a communication channel in the one or more physical media to bear a first portion of the device-detectable data; and (b) by causing another channel of the one or more physical media to bear a second portion of the device-detectable data.

351. The apparatus of clause 257 in which the one or more physical media have borne the device-detectable data.

352. The apparatus of clause 257 in which a portion of the one or more physical media comprises:

one or more static markings indicative of the device-detectable data.

353. The apparatus of clause 257 in which a portion of the one or more physical media comprises:

a magnetoresistive random access memory configured to receive the device-detectable data.

354. The apparatus of clause 257 further comprising at least one of a satellite dish or other signal-reflective element, a transducer, an antenna, or a receiver operated to receive the device-detectable data.

355. An apparatus comprising:

one or more physical media bearing a laser-scanned image of at least some of a cell to which an optical enhancement material was applied in vivo.

356. The apparatus of clause 355, further comprising:

a device having (at least) a handling control surface and a chamber, the chamber configured to receive (at least) the cell to which (at least) the optical enhancement material was applied (at least) in vivo.

357. The apparatus of clause 355 in which the laser-scanned image comprises:

output from a device positioned with a handling control surface.

358. The apparatus of clause 355 in which the laser-scanned image comprises:

a product of a noninvasive protocol.

359. The apparatus of clause 355 in which the laser-scanned image comprises:

a product of a minimally invasive protocol.

360. The apparatus of clause 355 in which the laser-scanned image comprises:

a product of a surgical protocol.

361. The apparatus of clause 355 in which the laser-scanned image comprises:

a product of an agricultural protocol.

362. The apparatus of clause 355 in which the laser-scanned image comprises:

image data depicting frozen tissue including the cell.

363. The apparatus of clause 355 in which the one or more physical media further comprises:

a signal from a surgical instrument that was positioned adjacent the cell.

364. The apparatus of clause 355 in which the one or more physical media further comprises:

an image of the cell from an electron microscope.

365. The apparatus of clause 355 in which the one or more physical media further comprises:

some of the one or more physical media bearing a signal from a biosensor.

366. The apparatus of clause 355 in which the one or more physical media further comprises:

a result of an in situ hybridization protocol performed upon the cell.

367. The apparatus of clause 355 in which the one or more physical media further comprises:

a result of positioning at least a component of the cell in a microfluidic structure.

368. The apparatus of clause 355 in which the one or more physical media further comprises:

some of the one or more physical media bearing a result of the optical enhancement material including one or more antibodies.

369. The apparatus of clause 355 in which the one or more physical media further comprises:

a result of the optical enhancement material applied in vivo indicating an absence of or a presence of a first attribute in the cell.

370. The apparatus of clause 355 in which the one or more physical media further comprises:

some of the one or more physical media bearing a signal received from one or more chemical sensors.

371. The apparatus of clause 355 in which the one or more physical media further comprises:

a karyotype of an organism to which the optical enhancement material was applied in vivo.

372. The apparatus of clause 355 in which the one or more physical media further comprises:

a data component relating to blood extracted from an organism to which the optical enhancement material was applied in vivo.

373. The apparatus of clause 355 in which the one or more physical media further comprises:

a data component relating to fluid extracted from the organism to which the optical enhancement material was applied in vivo.

374. The apparatus of clause 355 in which the one or more physical media further comprises:

an extraction protocol descriptor relating to the cell.

375. The apparatus of clause 355 in which the one or more physical media further comprises:

some of the one or more physical media bearing other data relating to the cell.

376. The apparatus of clause 355 in which the one or more physical media further comprises:

one or more identifiers of a protocol by which the optical enhancement material was applied to the cell in vivo.

377. The apparatus of clause 355 in which the laser-scanned image comprises:

the laser-scanned image depicting the cell frozen.

378. The apparatus of clause 355 in which the one or more physical media further comprises:

one or more identifiers of the optical enhancement material to which the cell was exposed in vivo.

379. The apparatus of clause 355 in which the one or more physical media further comprises:

one or more identifiers of a luminescent component of the optical enhancement material.

380. The apparatus of clause 355 in which the one or more physical media further comprises:

a go/no-go indication relating to the cell.

381. The apparatus of clause 355 in which the one or more physical media further comprises:

a go/no-go indication suggesting whether or not tissue containing the cell should be extracted.

382. The apparatus of clause 355 in which the one or more physical media further comprises:

another image of the cell generated after the laser-scanned image.

383. The apparatus of clause 355 in which the one or more physical media further comprises:

some of the one or more physical media indicating an extraction of the cell from an organism.

384. The apparatus of clause 355, further comprising:

circuitry for causing the optical enhancement material to be applied to the cell in vivo in response to contemporaneous user input.

385. The apparatus of clause 375, further comprising:

circuitry for causing at least some of the optical enhancement material to be applied to the cell in vivo within five seconds after a user's signal.

386. The apparatus of clause 355, further comprising:

circuitry for positioning a dispenser adjacent the cell in vivo.

387. The apparatus of clause 355, further comprising:

circuitry for processing device-detectable data obtained from one or more biomarker detection protocols performed upon the cell.

388. The apparatus of clause 355, further comprising:

circuitry for processing device-detectable data obtained from one or more protocols performed upon the cell in vivo.

389. The apparatus of clause 355, further comprising:

circuitry for processing device-detectable data obtained from one or more laser scanning protocols performed upon the cell.

390. The apparatus of clause 355, further comprising:

one or more other physical media bearing an operational setting value usable in laser scanning equipment for analyzing the cell.

391. The apparatus of clause 355 in which the one or more physical media comprises:

some of the one or more physical media bearing an operational setting value usable for analyzing the cell.

392. The apparatus of clause 355 in which the one or more physical media comprises:

a conduit configured to bear a result of an irradiation in vivo of the cell to which the optical enhancement material was applied in vivo.

393. The apparatus of clause 355 in which the one or more physical media comprises:

one or more conduits coupling imaging equipment with a module configured to contain the cell to which the optical enhancement material was applied in vivo.

394. The apparatus of clause 355 in which the one or more physical media comprises:

one or more conduits (at least) coupling imaging equipment with (at least) an instrument (at least) configured to perform (at least) an extraction of (at least) the cell to which (at least) the optical enhancement material was applied (at least) in vivo.

395. The apparatus of clause 355 in which a portion of the one or more physical media comprises:

one or more quantifications derived from an optical field of the cell.

396. The apparatus of clause 355 in which a portion of the one or more physical media comprises:

an attribute of a macromolecule relating to an organism to which the optical enhancement material was applied in vivo.

397. The apparatus of clause 355 in which a portion of the one or more physical media comprises:

a shape-indicative category relating to a portion of the laser-scanned image.

398. The apparatus of clause 355 in which a portion of the one or more physical media comprises:

a go/no-go indication of whether the cell apparently exhibits an attribute of interest.

399. The apparatus of clause 355 in which a portion of the one or more physical media comprises:

a portion of the laser-scanned image indicating a luminescent marking agent in the optical enhancement material.

400. The apparatus of clause 355 in which a portion of the one or more physical media comprises:

one or more size-descriptive quantities relating to the cell.

401. The apparatus of clause 355, further comprising:

an extraction module configured to contain the cell to which the optical enhancement material was applied in vivo.

402. The apparatus of clause 355, further comprising:

one or more lenses configured to receive optical energy from a region containing the cell; and

circuitry for transforming a portion of the optical energy into the laser-scanned image.

403. The apparatus of clause 355, further comprising:

a dispenser of the optical enhancement material.

404. The apparatus of clause 355 in which the one or more physical media comprise:

a portable module including at least an auditory interface configured to be operated while the portable module is held or worn.

405. The apparatus of clause 355 in which a portion of the one or more physical media comprises:

an image projection module.

406. The apparatus of clause 355 in which a portion of the one or more physical media comprises:

a touch screen.

407. The apparatus of clause 355 in which the one or more physical media include at least one of a repeater, a communication satellite, or another active module configured to accept first and second portions of the laser-scanned image at first and second respective times.

408. The apparatus of clause 355 in which a portion of the one or more physical media comprises:

one or more processors configured to perform optical image scanning upon the laser-scanned image.

409. The apparatus of clause 355 in which a portion of the one or more physical media comprises:

one or more processors configured to perform optical character recognition upon the laser-scanned image.

410. The apparatus of clause 355 in which a portion of the one or more physical media comprises:

circuitry for using an encryption constraint in at least some of the laser-scanned image.

411. The apparatus of clause 355 in which at least one of the one or more physical media comprises:

one or more signal-bearing media bearing at least one of a special-purpose instruction sequence or an information-bearing static attribute.

412. The apparatus of clause 355 in which a first portion of the one or more physical media transmits a portion of the laser-scanned image before a remainder of the one or more physical media transmits a remainder of the laser-scanned image.

413. The apparatus of clause 355 in which the one or more physical media include at least one of an integrated circuit, a data-holding element, a lens or other light-transmissive medium, a signal-bearing conduit currently bearing at least a portion of the laser-scanned image, or a bus or other configuration of two or more transmission media in mutual isolation.

414. The apparatus of clause 355 in which a portion of the one or more physical media comprises:

a power line operated for transmitting content of the laser-scanned image between at least two terminals.

415. The apparatus of clause 355 in which a first medium of the one or more physical media bears a first portion of the laser-scanned image while a second medium of the one or more physical media bears a second portion of the laser-scanned image.

416. The apparatus of clause 355 in which the one or more physical media are configured at least (a) by causing a communication channel in the one or more physical media to bear a first portion of the laser-scanned image; and (b) by causing another channel of the one or more physical media to bear a second portion of the laser-scanned image.

417. The apparatus of clause 355 in which the one or more physical media have borne the laser-scanned image.

418. The apparatus of clause 355 in which a portion of the one or more physical media comprises:

one or more static markings indicative of the laser-scanned image.

419. The apparatus of clause 355 in which a portion of the one or more physical media comprises:

a magnetoresistive random access memory configured to receive the laser-scanned image.

420. The apparatus of clause 355 further comprising at least one of a satellite dish or other signal-reflective element, a transducer, an antenna, or a receiver operated to receive the laser-scanned image.

421. An apparatus comprising:

one or more physical media bearing (a) an earlier image depicting at least some of a cell to which an optical enhancement material was applied in vivo and (b) a later image depicting at least some of the cell to which the optical enhancement material was applied in vivo.

422. The apparatus of clause 421 in which the one or more physical media further comprises:

some of the one or more physical media bearing other data relating to the cell.

423. The apparatus of clause 421 in which the one or more physical media further comprises:

one or more identifiers (at least) of a protocol by which (at least) the optical enhancement material was applied (at least) to the cell (at least) in vivo.

424. The apparatus of clause 421 in which the one or more physical media further comprises:

one or more identifiers of a protocol by which the cell was frozen.

425. The apparatus of clause 421 in which the one or more physical media further comprises:

one or more identifiers of the optical enhancement material to which the cell was exposed in vivo.

426. The apparatus of clause 421 in which the one or more physical media further comprises:

one or more identifiers of a luminescent component of the optical enhancement material.

427. The apparatus of clause 421 in which the one or more physical media further comprises:

a go/no-go indication relating to the cell.

428. The apparatus of clause 421 in which the one or more physical media further comprises:

a go/no-go indication suggesting whether tissue containing the cell should be extracted.

429. The apparatus of clause 421 in which the earlier image comprises:

a laser-scanned image of at least some of the cell to which the optical enhancement material was applied in vivo.

430. The apparatus of clause 421 in which the later image comprises:

the later image depicting the cell frozen.

431. The apparatus of clause 421 in which the one or more physical media further comprises:

some of the one or more physical media indicating an extraction of the cell from an organism.

432. The apparatus of clause 421, further comprising:

circuitry for causing the optical enhancement material to be applied to the cell in vivo in response to contemporaneous user input.

433. The apparatus of clause 432, further comprising:

circuitry for causing at least some of the optical enhancement material to be applied to the cell in vivo within five seconds after a user's signal.

434. The apparatus of clause 421, further comprising:

circuitry for positioning a dispenser adjacent the cell in vivo.

435. The apparatus of clause 421, further comprising:

circuitry for processing device-detectable data obtained from one or more biomarker detection protocols performed upon the cell.

436. The apparatus of clause 421, further comprising:

circuitry for processing device-detectable data obtained from one or more protocols performed upon the cell in vivo.

437. The apparatus of clause 421, further comprising:

circuitry for processing device-detectable data obtained from one or more laser scanning protocols performed upon the cell.

438. The apparatus of clause 421, further comprising:

one or more other physical media bearing an operational setting value usable in laser scanning equipment for analyzing the cell.

439. The apparatus of clause 421 in which the one or more physical media comprises:

some of the one or more physical media bearing an operational setting value usable for analyzing the cell.

440. The apparatus of clause 421 in which the one or more physical media comprises:

a conduit configured to bear a result of an irradiation in vivo of the cell to which the optical enhancement material was applied in vivo.

441. The apparatus of clause 421 in which the one or more physical media comprises:

one or more conduits coupling imaging equipment with a module configured to contain the cell to which the optical enhancement material was applied in vivo.

442. The apparatus of clause 421 in which the one or more physical media comprises:

one or more conduits coupling imaging equipment with an instrument configured to perform an extraction of the cell to which the optical enhancement material was applied in vivo.

443. The apparatus of clause 421 in which a portion of the one or more physical media comprises:

one or more quantifications derived from an optical field of the cell.

444. The apparatus of clause 421 in which a portion of the one or more physical media comprises:

an attribute of a macromolecule relating to tissue to which the optical enhancement material was applied in vivo.

445. The apparatus of clause 421 in which a portion of the one or more physical media comprises:

a shape-indicative category relating to a cell group containing the cell.

446. The apparatus of clause 421 in which a portion of the one or more physical media comprises:

a go/no-go indication of whether the cell apparently exhibits an attribute of interest.

447. The apparatus of clause 421 in which a portion of the one or more physical media comprises:

an indication of a luminescent marking agent in the optical enhancement material.

448. The apparatus of clause 421 in which a portion of the one or more physical media comprises:

one or more size-descriptive quantities relating to the cell.

449. The apparatus of clause 421 in which a portion of the one or more physical media comprises:

at least some device-detectable data indicating a luminescent marking agent in a treatment of the cell.

450. The apparatus of clause 421 in which a portion of the one or more physical media comprises:

at least some device-detectable data indicating a stain in a treatment of the cell.

451. The apparatus of clause 421 in which a portion of the one or more physical media comprises:

one or more size-descriptive quantities relating to the cell.

452. The apparatus of clause 421 in which the one or more physical media comprise:

at least one of the one or more physical media bearing device-detectable data that was generated while a treatment was applied to the cell.

453. The apparatus of clause 421 in which the one or more physical media comprise:

at least one of the one or more physical media bearing device-detectable data that was generated after a chamber was withdrawn from an organism to which the optical enhancement material was applied in vivo.

454. The apparatus of clause 421 in which the one or more physical media comprise:

at least one of the one or more physical media bearing a result that was generated from raw sensor data.

455. The apparatus of clause 421 in which the one or more physical media comprise:

at least one of the one or more physical media bearing device-detectable data that was generated while a chamber extended into an organism to which the optical enhancement material was applied in vivo.

456. The apparatus of clause 421, further comprising:

an extraction module containing a chamber, in which a treatment commenced upon a portion of an organism's tissue in the chamber and continued upon the cell in the chamber.

457. The apparatus of clause 421, further comprising:

an extraction module containing a chamber, in which the chamber contained a reagent to begin a treatment upon tissue entering the chamber.

458. The apparatus of clause 421, further comprising:

a laser microtome configured to extract a portion of the cell by severing a portion of tissue in a chamber from a remainder of the tissue in the chamber.

459. The apparatus of clause 421, further comprising:

an instrument configured to observe the cell in a chamber and to transmit at least some device-detectable data on the one or more physical media.

460. The apparatus of clause 421, further comprising:

an instrument configured to observe the cell in a chamber and to store at least some device-detectable data on the one or more physical media.

461. The apparatus of clause 421, further comprising:

an instrument configured to observe the cell in a chamber and to present at least some device-detectable data on the one or more physical media.

462. The apparatus of clause 421, further comprising:

an electron microscope configured to observe the cell in a chamber and to provide at least some device-detectable data on the one or more physical media.

463. The apparatus of clause 421, further comprising:

a fluorescence microscope configured to observe the cell in a chamber and to provide at least some device-detectable data on the one or more physical media.

464. The apparatus of clause 421, further comprising:

a confocal microscope configured to observe the cell in a chamber and to provide at least some device-detectable data on the one or more physical media.

465. The apparatus of clause 421, further comprising:

a spectrometer configured to observe the cell in a chamber and to provide at least some device-detectable data on the one or more physical media.

466. The apparatus of clause 421, further comprising:

an imaging system configured to observe the cell in a chamber and to provide at least some device-detectable data on the one or more physical media.

467. The apparatus of clause 421, further comprising:

a nuclear magnetic resonance imaging system configured to observe the cell in a chamber and to provide at least some device-detectable data on the one or more physical media.

468. The apparatus of clause 421, further comprising:

circuitry for transmitting energy into the cell in a chamber; and

circuitry for capturing an image of the cell.

469. The apparatus of clause 421, further comprising:

a surgical instrument with a handling control surface; and

an extraction module containing a chamber and supportable by and separable from the surgical instrument.

470. The apparatus of clause 421, further comprising:

a handling control surface configured to permit a user to extend an entirety of a chamber into an organism.

471. The apparatus of clause 421, further comprising:

at least one of the one or more physical media bearing a descriptor of an instrument that contains at least one chamber configured to contain at least the cell.

472. The apparatus of clause 421, further comprising:

at least some device-detectable data indicating a therapeutic agent administered to an organism to which the optical enhancement material was later applied in vivo.

473. The apparatus of clause 421, further comprising:

at least some device-detectable data (at least) indicating (at least) a therapeutic agent (at least) administered (at least) to the cell (at least) in vivo.

474. The apparatus of clause 421 in which the one or more physical media comprise:

a portable module including at least an auditory interface configured to be operated while the portable module is held or worn.

475. The apparatus of clause 421 in which a portion of the one or more physical media comprises:

an image projection module.

476. The apparatus of clause 421 in which a portion of the one or more physical media comprises:

a touch screen.

477. The apparatus of clause 421 in which the one or more physical media include at least one of a repeater, a communication satellite, or another active module configured to accept first and second portions of the earlier image at first and second respective times.

478. The apparatus of clause 421 in which a portion of the one or more physical media comprises:

one or more processors configured to perform one or more of image scanning or auditory pattern scanning upon device-detectable data relating to an organism to which the optical enhancement material was applied in vivo.

479. The apparatus of clause 421 in which a portion of the one or more physical media comprises:

one or more processors configured to perform linguistic pattern scanning upon device-detectable data relating to an organism to which the optical enhancement material was applied in vivo.

480. The apparatus of clause 421 in which a portion of the one or more physical media comprises:

circuitry for using an encryption constraint in at least some device-detectable data.

481. The apparatus of clause 421 in which at least one of the one or more physical media comprises:

one or more signal-bearing media bearing at least one of a special-purpose instruction sequence or an information-bearing static attribute as device-detectable data.

482. The apparatus of clause 421 in which a first portion of the one or more physical media transmits a portion of the earlier image before a remainder of the one or more physical media transmits a remainder of the earlier image.

483. The apparatus of clause 421 in which the one or more physical media include at least one of an integrated circuit, a data-holding element, a lens or other light-transmissive medium, a signal-bearing conduit currently bearing at least device-detectable data, or a bus or other configuration of two or more transmission media in mutual isolation.

484. The apparatus of clause 421 in which a portion of the one or more physical media comprises:

a power line operated for transmitting content of the earlier image between at least two terminals.

485. The apparatus of clause 421 in which a first medium of the one or more physical media bears a first portion of the earlier image while a second medium of the one or more physical media bears a second portion of the earlier image.

486. The apparatus of clause 421 in which the one or more physical media are configured at least (a) by causing a communication channel in the one or more physical media to bear a first portion of the earlier image; and (b) by causing another channel of the one or more physical media to bear a second portion of the earlier image.

487. The apparatus of clause 421 in which the one or more physical media have borne the earlier image.

488. The apparatus of clause 421 in which a portion of the one or more physical media comprises:

one or more static markings indicative of an organism in which the optical enhancement material was applied in vivo.

489. The apparatus of clause 421 in which a portion of the one or more physical media comprises:

a magnetoresistive random access memory configured to receive the earlier image.

490. The apparatus of clause 421 further comprising at least one of a satellite dish or other signal-reflective element, a transducer, an antenna, or a receiver operated to receive the earlier image.

Although selected combinations of the respective clauses are indicated above, this is by way of illustration only, and all relevant combinations of the clauses is also envisaged herein.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A medical or veterinary system comprising:
first means for extracting a tissue sample into a surgical probe;
second means for applying a first treatment to the tissue sample in the surgical probe; and
third means for transmitting a result of the first treatment from the surgical probe.

2. A medical or veterinary system comprising:
a surgical probe having a first separable extraction module;
a treatment module configured to apply a first treatment to a tissue sample in the first separable extraction module of the surgical probe; and
an output module configured to transmit a result of the first treatment from the surgical probe.

3. The medical or veterinary system of claim 2 in which the treatment module comprises:
circuitry for causing an application of the first treatment in response to contemporaneous user input.

4. The medical or veterinary system of claim 2, further comprising:
circuitry for positioning at least the first separable extraction module.

5. The medical or veterinary system of claim 2, further comprising:
circuitry for positioning at least a distal end of the surgical probe.

6. The medical or veterinary system of claim 2, further comprising:
circuitry for processing data from one or more assay protocols performed upon the tissue sample.

7. The medical or veterinary system of claim 2, further comprising:
circuitry for processing data from one or more biomarker detection protocols performed upon the tissue sample.

8. The medical or veterinary system of claim 2, further comprising:
at least one medium bearing an operational setting value usable in laser scanning equipment for analyzing the tissue sample.

9. The medical or veterinary system of claim 2, further comprising:
at least one medium bearing an operational setting value usable in a mass spectroscope for analyzing the tissue sample.

10. The medical or veterinary system of claim 2, further comprising:
at least one medium bearing an operational setting value usable in a microscope for analyzing the tissue sample.

11. The medical or veterinary system of claim 2, further comprising:
laser scanning equipment operable for receiving the first separable extraction module.

12. The medical or veterinary system of claim 2, further comprising:
imaging equipment operable for receiving the first separable extraction module.

13. The medical or veterinary system of claim 2 in which the output module comprises:
one or more conduits operably coupling the first separable extraction module to imaging equipment operable to detect the result of the first treatment.

14. The medical or veterinary system of claim 2, further comprising:
circuitry for configuring the result to include one or more quantifications derived from an optical field of the surgical probe.

15-16. (canceled)

17. The medical or veterinary system of claim 2 in which the first treatment comprises:
a permeabilizing agent.

18. The medical or veterinary system of claim 2 in which the first treatment comprises:
one or more of a microinjection or an electropermeabilization.

19. The medical or veterinary system of claim 2, further comprising:
an ultramicrotome configured to section the tissue sample.

20. The medical or veterinary system of claim 2, further comprising:
a device configured to extract the tissue sample by severing a larger tissue sample.

21. The medical or veterinary system of claim 2, in which the output module comprises:
a device configured to observe the tissue sample in a first chamber of the first separable extraction module and to transmit the result via one or more conduits.

22. (canceled)

23. The medical or veterinary system of claim 2, further comprising:
a device configured to monitor the tissue sample in the first separable extraction module and to cause a presentation of at least some of the result on one or more physical media.

24. The medical or veterinary system of claim 2, further comprising:
an electron microscope configured to observe the tissue sample and to provide at least some of the result on one or more physical media.

25. The medical or veterinary system of claim 2, further comprising:
a fluorescence microscope configured to observe the tissue sample and to provide at least some of the result on one or more physical media.

26. The medical or veterinary system of claim 2, further comprising:
a confocal microscope configured to observe the tissue sample and to provide at least some of the result on one or more physical media.

27. The medical or veterinary system of claim 2, further comprising:
a spectrometer configured to observe the tissue sample and to provide at least some of the result on one or more physical media.

28. The medical or veterinary system of claim 2, further comprising:
an imaging system configured to observe the tissue sample and to provide at least some of the result on one or more physical media.

29. The medical or veterinary system of claim 2, further comprising:
a nuclear magnetic resonance imaging system configured to observe the tissue sample and to transmit at least some of the result on one or more physical media.

30. The medical or veterinary system of claim 2, further comprising:
circuitry for transmitting energy into the tissue sample; and
circuitry for capturing an image of the tissue sample.

31. The medical or veterinary system of claim 2 in which the surgical probe comprises:
one or more handling control surfaces configured to permit a user to extend an entirety of the first separable extraction module into an organism.

32. The medical or veterinary system of claim 2 in which the surgical probe comprises:
the first separable extraction module containing a chamber and supportable by and separable from a remainder of the surgical probe.

33. The medical or veterinary system of claim 2 in which the surgical probe comprises:
a second extraction module.

34. The medical or veterinary system of claim 2, further comprising:
one or more physical media bearing a descriptor of a device that includes at least the first separable extraction module.

35. The medical or veterinary system of claim 2, further comprising:
one or more media bearing an attribute of a macromolecule of the tissue sample.

36. The medical or veterinary system of claim 2, further comprising:
one or more media bearing a shape-indicative category relating to a portion of the tissue sample.

37. The medical or veterinary system of claim 2, further comprising:
circuitry for selecting a stain effective for indicating whether the tissue sample includes a chromosomal abnormality.

38. The medical or veterinary system of claim 2, further comprising:
circuitry for causing the tissue sample to come into contact with a stain effective for indicating whether the tissue sample apparently exhibits an attribute of interest.

39. The medical or veterinary system of claim 2, further comprising:
one or more media bearing an indication of whether the tissue sample apparently exhibits a chromosomal attribute of interest.

40. The medical or veterinary system of claim 2, in which the first dispenser comprises:
one or more media bearing an indication of how much of the tissue sample apparently exhibits a pathology.

41. The medical or veterinary system of claim 2, in which the first dispenser comprises:
one or more media bearing a go/no-go result.

42. The medical or veterinary system of claim 2, further comprising:
a dispenser operable to mark a portion of the tissue sample with a luminescent material.

43. The medical or veterinary system of claim 2, further comprising:
a dispenser operable to mark a portion of the tissue sample with a stain.

44. The medical or veterinary system of claim 2, further comprising:
circuitry for configuring the result to include one or more size-descriptive quantities relating to the tissue sample.
